(19) United States
(12) Patent Application Publication
(10) Pub. No.: US 2026/0078373 A1
(43) Pub. Date: Mar. 19, 2026

(54) Cyclic Antisense Therapeutics

(71) Applicant: ARNAY SCIENCES, LLC, Shrewsbury, MA (US)

(72) Inventors: Sudhir AGRAWAL, Shrewsbury, MA (US); Vinod VATHIPADIEKAL, Stoneham, MA (US)

(21) Appl. No.: 19/334,208

(22) Filed: Sep. 19, 2025

Related U.S. Application Data
(63) Continuation of application No. PCT/US24/20914, filed on Mar. 21, 2024.
(60) Provisional application No. 63/453,893, filed on Mar. 22, 2023.
(60) Provisional application No. 63/454,235, filed on Mar. 23, 2023.

(51) Int. Cl. C12N 15/113; A61K 31/7105; A61K 31/711; A61K 31/712; A61K 31/7125

(52) U.S. Cl. CPC C12N 15/113; A61K 31/7105; A61K 31/711; A61K 31/712; A61K 31/7125; C12N 2310/11; C12N 2310/315; C12N 2310/321; C12N 2310/346; C12N 2310/3525; C12N 2310/532

(57) ABSTRACT

The present invention provides oligonucleotides referred to as cyclic structured oligonucleotides (“CSOs”) comprising a functional domain, a cyclizing domain, and a linker segment as described herein, compositions comprising same, and methods of using same. This design of cyclic oligonucleotides maintains a cyclic form until it is in the presence of and hybridizes with a targeted RNA.

### RELATED APPLICATIONS

This application is a continuation of International Application No. PCT/US24/20914, which designated the United States and was filed on Mar. 21, 2024, published in English, which claims the benefit of U.S. Provisional Application No. 63/453,893, filed on Mar. 22, 2023 and U.S. Provisional Application No. 63/454,235, filed on Mar. 23, 2023. The entire teachings of the above applications are incorporated herein by reference.

### SEQUENCE LISTING

The sequence listing submitted via EFS, in compliance with 37 CFR § 1.52 (e) (5), is incorporated herein by reference. The sequence listing XML file submitted via EFS contains the file “42503004US2SUBSEQ.xml”, created on Dec. 5, 2025, which is 405,031 bytes in size.

### BACKGROUND

Processing and Translation of targeted RNA can be modulated by antisense oligonucleotides by multiple mechanisms. These include cleaving the targeted RNA by RNase-H, modulating aberrant splicing, processing of targeted RNA and increased translation, inhibiting the translation by steric hindrance, etc. Targeted RNA could be mRNA or noncoding RNA. In other mechanisms, the antisense strand of a duplex siRNA could be incorporated in AGO and inhibit translation by the siRNA mechanism. In other mechanisms, like ADAR or CRISPR based models, antisense could edit RNA or DNA and thereby modulate translation and processing.

Over the years it has been learned that antisense hybridization and affinity influence selectivity for the targeted RNA. In addition, to use antisense nucleic acids as drugs, nuclease stability is important which has been provided by the modification of internucleotide linkages, for example, phosphorothioate.

It was postulated that nuclease stability is key for potency and since the degradation of antisense was shown to be from the 3′-end, the focus was to modify the 3′-end to slow down degradation. These designs include capping on the 3′-end, and a hairpin loop on the 3′-end, creating oligos having secondary structures comprising 3′-3′ linkages or attaching two (2) antisense oligonucleotides at their 3′ ends. These type of antisense showed increased nuclease stability but antisense potency was not improved. Unfortunately, these modifications also increased inflammatory responses thereby limiting the therapeutic index.

Both DNA and RNA and 2′-substituted RNA containing phosphorothioate have been studied as antisense agents and provide different characteristics. DNA phosphorothioate antisense, when hybridized to RNA, activates RNase H, whereas RNA or 2′-substituted RNA antisense binds to RNA with higher affinity and does not activate RNase H. To further improve antisense characteristics, a mixture of these two modifications has been employed in antisense, generally referred to as hybrid or gapmer antisense. In most studied gapmer antisense, modified RNA segment is placed on both the 3′- and 5′-end whereas DNA is placed in the middle. Gapmer antisense is the most widely studied antisense and drugs employing this chemistry are approved and are in clinical development.

One of the side-effects of both DNA and RNA phosphorothioate is due to the interaction with proteins and more specifically with the family of Pattern Recognition Receptors (PRRs). These interactions result in induction of an immune cascade thereby causing an off-target mechanism of action and related safety signals. Detailed structure-activity relationship studies have shown that the accessibility of the 5′-end of DNA and RNA phosphorothioate antisense is required for immune activation. DNA and RNA phosphorothioate containing two 5′-ends have shown increased immuno-stimulatory activity. In contrast, it has been previously shown that DNA and RNA phosphorothioate which contain two 3′-ends (and lack 5′-) show minimal inflammatory responses.

In continuing efforts to improve the properties of DNA and modified RNA phosphorothioate antisense as therapeutic agents, structural changes in oligonucleotides were considered. For example, in earlier studies, self-stabilized oligonucleotides were reported wherein oligodeoxynucleotide phosphorothioates (“PS-oligonucleotide”) containing a hairpin loop region at the 3′-end provided increased in vivo nuclease stability and limited biological activity. To date focus has been to improve stability of antisense by modifying 3′-end by various modifications including in gapmer antisense.

Despite the advances that have been made, there is still a desire to develop antisense oligonucleotides having improved properties for use as therapeutic agents and in diagnostic applications.

### SUMMARY OF THE INVENTION

The present invention provides a structural class of oligonucleotides referred to herein as “cyclic structured oligonucleotides” (CSOs) or, equivalently, “cyclic oligos”. In CSOs, two oligonucleotides are linked to each other (directly or through a linker segment), wherein one oligonucleotide of the CSO, referred to as the “functional domain,” provides a function to the CSO (e.g., the functional segment can be an antisense oligonucleotide or an immunostimulatory oligonucleotide), and the second oligonucleotide, referred to as the “cyclizing domain” comprises a nucleotide sequence that is complementary to a portion of the functional domain (e.g., FIG. 1A-FIG. 1C). Preferably, the cyclizing domain is complementary to nucleotides at a terminal end of the functional domain. Preferably, the cyclizing domain is complementary to an equal length portion of nucleotides at a terminal end of the functional domain.

CSOs adopt an intramolecular cyclic structure as a result of complementarity between functional and cyclizing domains, which form an intramolecular duplex. This intramolecular duplex formation changes both the shape of the functional domain and accessibility to the ends of oligonucleotide. This structure combines key attributes to create optimal antisense and oligonucleotide and nucleic acid based therapeutics.

In gene and RNA expression modulation, this structure masks the 5′-end thereby reducing the interaction with PRRs and permitting endosomal escape. Once in the cytoplasm or nucleus, the cyclic structure will open in the presence of target RNA as the affinity of the functional domain and a target RNA sequence is higher than the affinity between the functional domain and cyclizing domain. When the CSO is in the intramolecular cyclic form, it may exhibit fewer of the polyanionic-related side effects (e.g., complement activation and prolongation of partial thromboplastin time) known to occur with PS-oligonucleotides, because there are fewer exposed phosphorothioate linkages. Also, CSOs would have reduced protein binding.

CSOs according to the invention can be made using standard techniques for synthesis of the constituent oligonucleotides and are useful for all purposes for which the functional oligonucleotide and nucleic acid is useful.

The foregoing merely summarizes certain aspects of the invention and is not intended, nor should it be construed, as limiting the invention in any manner. All patents, patent applications, and other publications recited in this specification are hereby incorporated by reference in their entirety.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A through FIG. 1C depicts various embodiments of the cyclic structured oligonucleotide according to the invention. The dashed line represents the cyclizing domain. The solid line represents the functional domain. L represents the linking of the functional domain and cyclizing domain directly or via a linker. As shown in FIG. 1A, the cyclic structured oligonucleotide maintains a cyclic form until it is in the presence of and hybridizes with a targeted RNA. In this embodiment, the functional domain is antisense oligonucleotide which is complementary to the targeted RNA. The cyclic structured oligonucleotides can act by various mechanisms of action depending on the nature of the oligonucleotide of the functional domain as will be further described herein. FIG. 1B depicts one embodiment of a CSO according to the invention wherein the oligonucleotide of the functional domain is a splitmer as described herein.

FIG. 1C depicts one embodiment of a CSO according to the invention wherein the oligonucleotide of the functional domain is a splicing oligonucleotide as described herein.

FIG. 2 depicts the delivery of a CSO according to the invention wherein the oligonucleotide of the functional domain is a gene modulation oligonucleotide, and its release from the endosome into the cytoplasm.

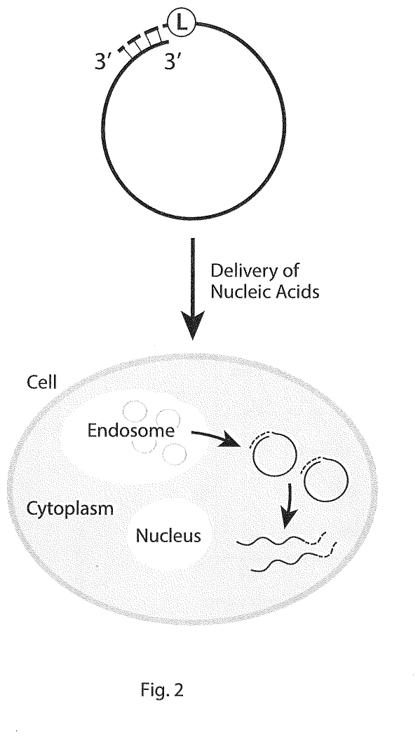

FIG. 3A and FIG. 3B depict the knockdown of angiopoietin-like protein 3 (ANGPTL3) in Hep3B cells. The underlined segment of the gapmer control oligonucleotide represents 2′-OME nucleotides.

FIG. 4A and FIG. 4B depict the knockdown of apolipoprotein B (ApoB) in Hep3B cells. The underlined segment of the gapmer control oligonucleotide represents 2′-OME nucleotides.

FIG. 5A and FIG. 5B depict the knockdown of apolipoprotein C-III (APOC3) in Hep3B cells. The underlined segment of the gapmer control oligonucleotide represents 2′-OME nucleotides.

FIG. 6A and FIG. 6B depict the knockdown of diacylglycerol O-acyltransferase 2 (DGAT2) in Hep3B cells. The underlined segment of the gapmer control oligonucleotide represents 2′-OME nucleotides.

FIG. 7A and FIG. 7B depict the knockdown of kallikrein B1 (KLKB1) in Hep3B cells. The underlined segment of the gapmer control oligonucleotide represents 2′-OME nucleotides.

FIG. 8A and FIG. 8B depict the knockdown of proprotein convertase subtilisin/kexin type 9 (PCSK9) in Hep3B cells. The underlined segment of the gapmer control oligonucleotide represents 2′-OME nucleotides.

FIG. 9A and FIG. 9B depict the knockdown of protein-tyrosine phosphatase 1B (PTP1B) in Hep3B cells. The underlined segment of the gapmer control oligonucleotide represents 2′-OME nucleotides.

FIG. 10A and FIG. 10B depict the knockdown of signal transducer and activator of transcription 3 (STAT3) in Hep3B cells. The underlined segment of the gapmer control oligonucleotide represents 2′-OME nucleotides.

FIG. 11A and FIG. 11B depict the knockdown of transthyretin (TTR) in Hep3B cells. The underlined segment of the gapmer control oligonucleotide represents 2′-OME nucleotides.

FIG. 12A and FIG. 12B depict the knockdown of superoxide dismutase 1 (SOD1) in Hep3B cells.

FIG. 13A and FIG. 13B depict the knockdown of Huntingtin (HTT) in U-251 MG cells. In the gapmer control oligonucleotide, “e” represents 2′-MOE nucleotides and the “*” represents a phosphorothioate internucleotide linkage.

FIG. 14A and FIG. 14B depict the knockdown of Huntingtin (HTT) in U-251 MG cells. In the gapmer control oligonucleotide, “e” represents 2′-MOE nucleotides and the “*” represents a phosphorothioate internucleotide linkage.

FIG. 15A and FIG. 15B depict the knockdown of microtubule associated protein tau (MAPT) in U-251 MG cells. In the gapmer control oligonucleotide, “e” represents 2′-MOE nucleotides and the “*” represents a phosphorothioate internucleotide linkage.

FIG. 16A and FIG. 16B depict the knockdown of superoxide dismutase 1 (SOD1) in U-251 MG cells. In the gapmer control oligonucleotide, “e” represents 2′-MOE nucleotides and the represents a phosphorothioate internucleotide linkage.

FIG. 17A and FIG. 17B depict the knockdown of apolipoprotein C-III (APOC3) in Hep3B cells. The underlined segment of the gapmer control oligonucleotide represents 2′-OME nucleotides.

FIG. 18A and FIG. 18B depict the knockdown of apolipoprotein C-III (APOC3) in Hep3B cells. The underlined segment of the gapmer control oligonucleotide represents 2′-OME nucleotides.

### DETAILED DESCRIPTION

It is to be understood that both the foregoing general description and the following detailed description are exemplary and explanatory only and are not restrictive.

Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers with that range, for example, 1, 2, 2.7, 3, 4, 5, 5.3, and 6. This applies regardless of the breadth of the range.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of +20% or +10%, including+5%, +1%, and +0.1% from the specified value, as such variations are appropriate to perform the disclosed methods.

The present invention provides oligonucleotides referred to as cyclic structured oligonucleotides (“CSOs”) comprising a functional domain, a cyclizing domain, and a linker segment. Unless noted otherwise, the functional domain and the cyclizing domain are linked at their 5′ ends via a 5′-5′ linkage. Alternatively, in some embodiments, the functional domain and the cyclizing domain are linked at their 3′ ends via a 3′-3′ linkage.

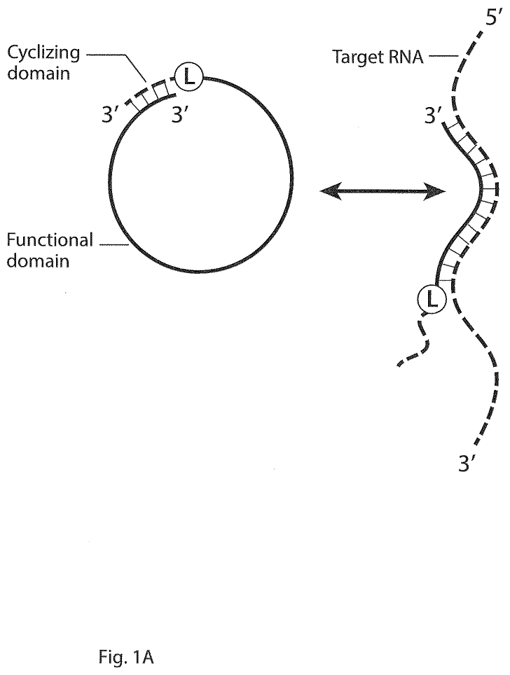

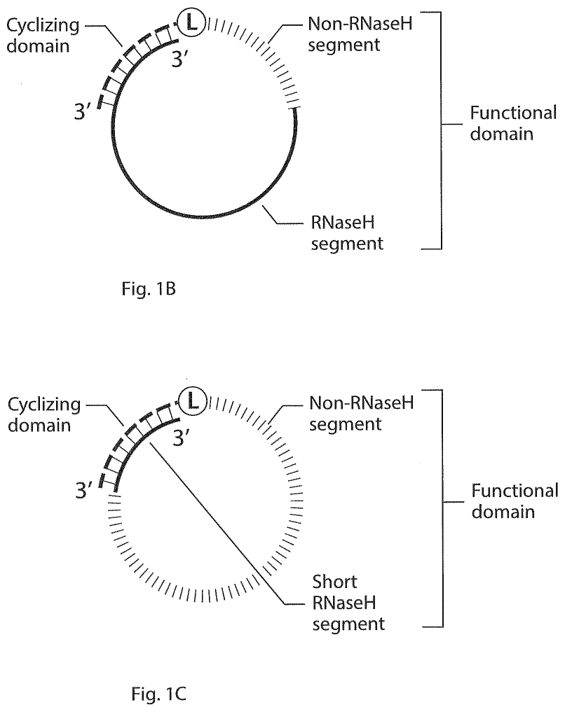

In embodiments, the cyclizing domain is attached to the functional domain on the 5′-end with a 5′-5′ linkage. In this configuration, the cyclizing domain hybridizes with the 3′-end of the oligonucleotide of the functional domain, thereby forming a cyclic structure (e.g., FIG. 1A-FIG. 1C). This design of cyclic oligonucleotides maintains a cyclic form until it is in the presence of and the functional domain hybridizes with a targeted RNA. This structure allows for increased specificity.

In embodiments, the cyclizing domain is attached to the functional domain on the 3′-end with a 3′-3′ linkage. In this configuration, the cyclizing domain hybridizes with the 5′-end of the oligonucleotide of the functional domain, thereby forming a cyclic structure.

The functional domain provides a desired function to the CSO. For example, for gene expression modulation, the oligonucleotide of the functional domain is complementary to a targeted RNA.

In embodiments, the oligonucleotides of the functional domain and the cyclizing domain are DNA or RNA or combinations thereof. In embodiments, the oligonucleotide of the functional domain is DNA or RNA or combinations thereof. In embodiments, the oligonucleotide of the cyclizing domain is DNA or RNA or combinations thereof.

In embodiments, the oligonucleotides of the functional domain and/or the cyclizing domain are unmodified. In embodiments, the oligonucleotides of the functional domain are unmodified. In embodiments, the oligonucleotides of the cyclizing domain are unmodified. In embodiments, the oligonucleotides of the functional domain and the cyclizing domain are unmodified.

In embodiments, at least one nucleotide of the oligonucleotides of the functional domain and/or the cyclizing domain are modified. In embodiments, two or more nucleotides of the oligonucleotides of the functional domain and/or the cyclizing domain are modified.

In embodiments, the oligonucleotide of the functional domain is modified. In embodiments, the oligonucleotide of the functional domain comprises a modification of the inter-nucleotide linkage, sugar, heterocyclic base, or a combination thereof. These modifications could also be appropriately placed at specific positions within the oligonucleotide of the functional domain. Other chemistries and modification are known in the field of oligonucleotides that can be readily used in accordance with the disclosure and are encompassed within the term ‘modified’ as used in the context of an oligonucleotide herein. As used herein, the terms “oligonucleotide of the functional domain” or the “functional domain” are used interchangeably.

In embodiments, the functional domain comprises an oligonucleotide between 15 and 50 nucleotides in length. In embodiments, the functional domain comprises an oligonucleotide between 17 and 40 nucleotides in length. In embodiments, the functional domain comprises an oligonucleotide between 17 and 25 nucleotides in length. In embodiments, the oligonucleotide of the functional domain is between 17 and 22 nucleotides in length.

In embodiments, the oligonucleotide of the functional domain is 17, 18, 19, 20, 21, 22, 23, 24, 25, 26, 27, 28, 29, 30, 31, 32, 33, 34, 35, 36, 37, 38, 39, or 40 nucleotides in length. In embodiments, the functional domain is 17 nucleotides in length. In embodiments, the functional domain is 18 nucleotides in length. In embodiments, the functional domain is 19 nucleotides in length. In embodiments, the functional domain is 20 nucleotides in length. In embodiments, the functional domain is 21 nucleotides in length. In embodiments, the functional domain is 22 nucleotides in length. In embodiments, the functional domain is 23 nucleotides in length. In embodiments, the functional domain is 24 nucleotides in length. In embodiments, the functional domain is 25 nucleotides in length. In embodiments, the functional domain is 26 nucleotides in length. In embodiments, the functional domain is 27 nucleotides in length. In embodiments, the functional domain is 28 nucleotides in length. In embodiments, the functional domain is 29 nucleotides in length. In embodiments, the functional domain is 30 nucleotides in length. In embodiments, the functional domain is 31 nucleotides in length. In embodiments, the functional domain is 32 nucleotides in length. In embodiments, the functional domain is 33 nucleotides in length. In embodiments, the functional domain is 34 nucleotides in length. In embodiments, the functional domain is 35 nucleotides in length. In embodiments, the functional domain is 36 nucleotides in length. In embodiments, the functional domain is 37 nucleotides in length. In embodiments, the functional domain is 38 nucleotides in length. In embodiments, the functional domain is 39 nucleotides in length. In embodiments, the functional domain is 40 nucleotides in length.

In embodiments, the functional domain includes, but is not limited to, an oligonucleotide selected from an antisense oligonucleotide, a microRNA (miRNA), an siRNA, a piRNA, an hnRNA, an ncRNA, an snRNA, an sgRNA, an esiRNA, an shRNA, a lncRNA, a CRISPR-based system, an adenosine deaminase acting on RNA (ADAR) system, or a splicing oligonucleotide. In embodiments, the functional domain includes, but is not limited to, an oligonucleotide selected from an immunostimulatory oligonucleotide or an immune-inhibitory oligonucleotide, also referred to as an immune antagonist oligonucleotide.

The only limitation on the nucleotides and internucleotide linkages of the oligonucleotide of the functional domain is that they do not eliminate (a) the ability of the cyclizing domain to hybridize to and form a duplex with the functional domain of the CSO under the desired conditions, and (b) the ability of the functional domain to carry out its intended function (e.g., in the case of a functional domain that is an antisense oligonucleotide, to hybridize to and form a duplex with a complementary RNA segment under physiological conditions, which duplex is a substrate for RNase H). Preferred nucleotides and internucleotide linkages are those that will enhance the stability of the CSO to nucleases and other forms of chemical degradation and/or enhance the ability of the functional domain to carry out its intended function.

In embodiments, the internucleotide linkages of the functional domain are phosphorothioate internucleotide linkages, phosphorodithioate internucleotide linkages, phosphodiester internucleotide linkages or a combination thereof.

The oligonucleotide of cyclizing domain is complementary to a sequence of nucleotides within the functional domain and of polarity opposite to the sequence of nucleotides in the functional domain to which it is complementary.

In embodiments, the oligonucleotide of the cyclizing domain is modified. In embodiments, the oligonucleotide of the cyclizing domain comprises a modification of the inter-nucleotide linkage, sugar, heterocyclic base, or a combination thereof. These modifications could also be placed at specific positions within the oligonucleotide of the cyclizing domain. As used herein, the terms “oligonucleotide of the cyclizing domain” or the “cyclizing domain” are used interchangeably.

In embodiments, the internucleotide linkages of the cyclizing domain are phosphorothioate internucleotide linkages, phosphodiester internucleotide linkages or a combination thereof.

In embodiments, the cyclizing domain comprises an oligonucleotide between 4 and 30 nucleotides in length. In embodiments, the cyclizing domain comprises an oligonucleotide between 4 and 25 nucleotides in length. In embodiments, the cyclizing domain comprises an oligonucleotide between 4 and 12 nucleotides in length. In embodiments, the cyclizing domain comprises an oligonucleotide between 4 and 10 nucleotides in length. In embodiments, the cyclizing domain comprises an oligonucleotide between 4 and 8 nucleotides in length. In embodiments, the cyclizing domain comprises an oligonucleotide between 4 and 6 nucleotides in length.

In embodiments, the cyclizing domain comprises an oligonucleotide between 5 and 8 nucleotides in length. In embodiments, the oligonucleotide of the cyclizing domain is 4, 5, 6, 7, 8, 9 or 10 nucleotides in length. In embodiments, the oligonucleotide of the cyclizing domain is 4 nucleotides in length. In embodiments, the oligonucleotide of the cyclizing domain is 5 nucleotides in length. In embodiments, the oligonucleotide of the cyclizing domain is 6 nucleotides in length. In embodiments, the oligonucleotide of the cyclizing domain is 7 nucleotides in length. In embodiments, the oligonucleotide of the cyclizing domain is 8 nucleotides in length. In embodiments, the oligonucleotide of the cyclizing domain is 9 nucleotides in length. In embodiments, the oligonucleotide of the cyclizing domain is 10 nucleotides in length.

As used herein, the term “polarity” refers to the concept of directionality in primary structure (e.g., 3′→5′ and 5′→3′ in the case of DNA and RNA, or N-terminal→C-terminal (or vice versa) in the case of PNAs). In the case where the CSOs of the invention comprise oligonucleotides, for example, which hybridize by Watson-Crick base pairing in anti-parallel fashion, the cyclizing domain will be in the 5′→3′ (or 2′) configuration and the sequence of nucleotides to which it is complementary in the functional domain will be in the 3′ (or 2′)→5′ configuration. The change in polarity in the CSO can occur anywhere in the CSO other than in the cyclizing domain and the sequence of nucleotides in the functional domain to which the cyclizing domain is complementary. In a preferred embodiment where the CSO comprises oligonucleotides, the functional domain is in the 3′→5′ configuration and the cyclizing domain is in the 5′→3′ configuration, such that the functional domain and the cyclizing domain are linked via a 5′-5′ linkage. In some embodiments, the functional domain is in the 5′→3′ configuration and the cyclizing domain is in the 3′→5′ configuration, such that the functional domain and the cyclizing domain are linked via a 3′-3′ linkage.

In embodiments, the functional domain and the cyclizing domain are covalently linked to each other through the linker. In embodiments, the linker segment is a direct bond, a nucleotide or oligonucleotide between 2 and 5 nucleotides in length, or other chemical moiety, or combinations thereof. In some embodiments, the linker segment can be cleavable.

The only limitation on the linker segment is that it does not eliminate the essential functions of the CSO, namely (a) the ability of the CSO to form an intramolecular cyclic structure under the conditions of interest (e.g., physiological conditions) and (b) the ability of the functional domain to carry out its intended function. Preferred “other chemical moiety” linkers include, but are not limited to, C₂-C₆ alkyl, ethylene glycol, tri (ethylene glycol), tetra (ethylene glycol), penta (ethylene glycol), hexa (ethylene glycol) and —NH(CH₂)ₙNH—, wherein n is 2, 3, 4, 5, or 6. Alternatively, the linker segment can be a combination of the foregoing. In a preferred embodiment, the linker is a direct bond, in which case the functional and cyclizing domains are directly bound. In an embodiment, the linker is ethylene glycol. In embodiments, the linker is a C₂-C₆ alkyl. In embodiments, the linker is a C₂ alkyl. In embodiments, the linker is a C₃ alkyl. In embodiments, the linker is a C₄ alkyl. In embodiments, the linker is a C₅ alkyl. In embodiments, the linker is a C₆ alkyl.

In embodiments, the linker is a branched linker, a fatty acid, or a lipid.

In the CSOs as described herein, the oligonucleotide of the functional domain has a terminal end and a linker end. As the name implies, the linker end is the end of the oligonucleotide linked to the cyclizing domain through the linker segment. In general, the CSO is constructed so the terminal end of the functional domain will form a duplex with the cyclizing domain, i.e., the cyclizing domain is complementary to the terminal end of the functional domain.

In embodiments, the nucleotides of the cyclizing domain are least 90% complementary over its entire length to a portion of oligonucleotide of the functional domain. In embodiments, the nucleotides of the cyclizing domain are least 95% complementary over its entire length to a portion of oligonucleotide of the functional domain. In embodiments, the nucleotides of the cyclizing domain are least 97% complementary over its entire length to a portion of oligonucleotide of the functional domain. In embodiments, the nucleotides of the cyclizing domain are least 98% complementary over its entire length to a portion of oligonucleotide of the functional domain. In embodiments, the nucleotides of the cyclizing domain are least 99% complementary over its entire length to a portion of oligonucleotide of the functional domain. In embodiments, the nucleotides of the cyclizing domain are least 100% complementary over its entire length to a portion of oligonucleotide of the functional domain.

When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mis-matched” and the strand is considered partially complementary. Accordingly, strands can be varying degrees of partially complementary (e.g., 0%<x<100% complementary), until no bases align, at which point they are non-complementary (e.g., 0% complementary). As is readily understood and recognized by one of skill in the art, full (i.e., complete, 100%) complementarity is not required for hybridization of strands of nucleic acids (e.g., oligonucleotides, antisense or otherwise).

In embodiments, the target RNA may be an mRNA, pre-mRNA, ncRNA, lncRNA, or microRNA. In embodiments, the target RNA is mRNA.

In embodiments, cyclic structured oligonucleotide according to the invention is part of a pharmaceutical composition comprising a pharmaceutically acceptable carrier.

The pharmaceutical composition comprising the cyclic structured oligonucleotide of the invention may further comprise any other agent or therapy useful for treating or preventing a disease or condition and does not diminish the gene expression modulation effect of the cyclic structured oligonucleotide according to the invention. Agent(s) useful for treating or preventing the disease or condition includes, but is not limited to, small molecules, peptides, vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the cyclic structured oligonucleotide according to the invention can be administered in combination with other compounds (for example formulated with lipids or liposomes, and conjugated to peptides, antibodies, or small molecules) to enhance the specificity or magnitude of the gene expression modulation of the cyclic structured oligonucleotide according to the invention.

In embodiments, the oligonucleotide of the functional domain comprises at least one phosphorothioate internucleotide linkage. In embodiments, at least half of the internucleotide linkages are phosphorothioate. In embodiments, all of the internucleotide linkages are phosphorothioate.

In embodiments, the oligonucleotide of the functional domain is single-stranded.

In embodiments, the oligonucleotide of the functional domain is at least 90% complementary over its entire length to a portion of a target RNA. In embodiments, the oligonucleotide of the functional domain is at least 95% complementary over its entire length to a portion of the target RNA. In embodiments, the oligonucleotide of the functional domain is at least 97% complementary over its entire length to a portion of the target RNA. In embodiments, the oligonucleotide of the functional domain is at least 98% complementary over its entire length to a portion of the target RNA. In embodiments, the oligonucleotide of the functional domain is at least 99% complementary over its entire length to a portion of the target RNA. In embodiments, the oligonucleotide of the functional domain is at least 100% complementary over its entire length to a portion of the target RNA.

### Functional Domains

In various embodiments the functional domain of the CSO is an oligonucleotide as further described below. The cyclizing domain and the linker of the CSO is as described above unless otherwise noted.

### Inhibition of Gene Expression

In embodiments, the invention provides a cyclic structured oligonucleotide (CSO) comprising a functional domain, a cyclizing domain, and a linker, wherein the functional domain and the cyclizing domain are linked at their 5′ ends, wherein the functional domain comprises an oligonucleotide between 15 and 45 nucleotides in length and is complementary to targeted RNA; wherein the cyclizing domain comprises an oligonucleotide between 4 and 12 nucleotides in length and is at least 90% complementary to a sequence of nucleotides within the functional domain and of polarity opposite to the sequence of nucleotides in the functional domain to which it is complementary; wherein functional domain comprises a gene modulating oligonucleotide. In embodiments, the oligonucleotide of the functional domain is modified.

In embodiments, the gene modulating oligonucleotide (i.e., an oligonucleotide that can modulate the expression of a target gene) of the functional domain includes, but is not limited to, an antisense oligonucleotide, a microRNA (miRNA), a piRNA, a hnRNA, a ncRNA, a snRNA, a sgRNA, an esiRNA, an shRNA, or a lncRNA.

In embodiments, the gene modulating oligonucleotide of the functional domain is an antisense oligonucleotide. In embodiments, the modification of the antisense oligonucleotide comprises at least one modified nucleobase, sugar and/or internucleotide linkage.

As appreciated by those skilled in the art, the CSOs of the invention may be utilized to improve oligonucleotides of prior technologies. The present disclosure provides improvements of prior technologies by applying the CSO as described herein to prior reported oligonucleotide base sequences, thereby providing improved compositions of previously reported oligonucleotides that may be useful for gene silencing.

Examples of previously reported antisense oligonucleotides useful as a functional domain within a cyclic structured oligonucleotide (CSO) according to the invention include, but are not limited to, the antisense oligonucleotides of Table 1.

TABLE 1

| Cmp # | Target | Sequence (SEQ ID NO:) |
|---|---|---|
| 280 | ApoB-100 | 5′-GCCTCAGTCTGCTTCGCACC-3′ (SEQ ID NO: 280) |
| 281 | TTR | 5′-TCTTGGTTACATGAAATCCC-3′ (SEQ ID NO: 281) |
| 282 | APOC+HD 3+L | 5′-AGCTTCTTGTCCAGCTTTAT-3′ (SEQ ID NO: 282) |
| 283 | PTP-1b | 5′-GCTCCTTCCACTGATCCTGC-3′ (SEQ ID NO: 283) |
| 284 | PCSK9 | 5′-CCTTGGCCACGCCGGCATCC-3′ (SEQ ID NO: 284) |
| 285 | STAT+HD 3+L | 5′-CTATTTGGATGTCAGC-3′ (SEQ ID NO: 285) |
| 286 | ANGPTL3 | 5′-GGACATTGCCAGTAATCGCA-3′ (SEQ ID NO: 286) |
| 287 | Kallikrein B1 | 5′-TGCAAGTCTCTTGGCAAACA-3′ (SEQ ID NO: 287) |
| 288 | DGAT+HD 2+L | 5′-TGCCATTTAATGAGCTTCAC-3′ (SEQ ID NO: 288) |
| 289 | SMN2 | 5′-TCACTTTCATAATGCTGG-3′ (SEQ ID NO: 289) |
| 290 | FUS | 3′-GCAAUGTCACCTTTCAUACC-5′ (SEQ ID NO: 290) |
| 291 | APO(a) | 5′-TGCTCCGTTGGTGCTTGTTC-3′ (SEQ ID NO: 291) |
| 292 | HTT | 5′-CUCAGTAACATTGACACCAC-3′ (SEQ ID NO: 292) |
| 293 | HBV | 5′-GCAGAGGTGAAGCGAAGTGC-3′ (SEQ ID NO: 293) |
| 294 | Angiotensinogen | 5′-GGGCCTGTTTAGTCGC-3′ (SEQ ID NO: 294) |
| 295 | Factor XI | 5′-ACGGCATTGGTGCACAGTTT-3′ (SEQ ID NO: 295) |
| 296 | TMPRSS6 | 5′-CTTTATTCCAAAGGGCAGCT-3′ (SEQ ID NO: 296) |
| 297 | Angiotensinogen | 5′-CACAAACAAGCTGGTCGGTT-3′ (SEQ ID NO: 297) |
| 298 | MAPT | 5′-CCGTTTTCTTACCACCCT-3′ (SEQ ID NO: 298) |
| 299 | PTP1B (PTPN1) | 5′-ATGGTTTATTCCATGGCCA-3′ (SEQ ID NO: 299) |
| 300 | HTT | 5′-CUUUCAGACACGGGAACACGG-3′ (SEQ ID NO: 300) |
| 301 | ATXN2 | 5′-CTTCACATTTCGATCCAACA-3′ (SEQ ID NO: 301) |
| 302 | SCN1A | 5′-AGTTGGAGCAAGATTATC-3′ (SEQ ID NO: 302) |
| 303 | DMD | 5′-CAATGCCATCCTGGAGTTCCTG-3′ (SEQ ID NO: 303) |
| 304 | DMD | 5′-GTTGCCTCCGGTTCTGAAGGTGTTC-3′ (SEQ ID NO: |
| | | 304) |
| 305 | DMD | 5′-CTCCAACATCAAGGAAGATGGCATTTCTAG-3′ (SEQ |
| | | ID NO: 305) |
| 306 | SOD1 | 5′-CAGGATACATTTCTACAGCT-3′ (SEQ ID NO: 306) |

In embodiments, the antisense compounds of the functional domain may have a defined percent identity to a particular nucleotide sequence, SEQ ID NO, provided herein (e.g., Tables 1, 2 and 4), or portion thereof. As used herein, an antisense compound is identical to the sequence disclosed in Table 1 if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, of the functional domain are at least 70%, 75%, 80%, 85%, 90%, 95%, 96%, 97%, 98%, 99% or 100% identical to one or more of the antisense compounds or SEQ ID NOs, or a portion thereof, disclosed herein.

As shown herein, the CSOs of the invention comprising an antisense oligonucleotide linked to a cyclizing domain via 5′-5′ linkage surprisingly demonstrated increased potency. This observation was in complete contrast to earlier hypotheses that increased stability (e.g., 3′-3′ linked oligonucleotides) would translate to increased potency. Furthermore, the lack of a 5′-end would make antisense oligonucleotides of the CSO less inflammatory. This design permits antisense oligonucleotides to unfold to linear structure and to be active in cells where the target RNA is expressed.

In the case where the CSO is an antisense oligonucleotide, it is in cyclic form until it is in the presence of complementary target RNA where it adopts a linear form and binds to the target RNA. The changes from cyclic form to linear form could be confirmed by thermal melting and RNase H cleavage studies. In the linear form, the functional domain hybridizes (under physiological conditions, at a minimum) with the complementary target RNA to form a duplex. This duplex is a substrate for RNase H, and, in the presence of RNase H and under the proper conditions (e.g., physiological), the RNA strand of the duplex will be cleaved by the RNase H, thereby preventing expression.

CSOs comprising antisense oligonucleotide functional domains maintain activity in cell cultures. The advantage foreseen with these CSOs is that their formation of intramolecular cyclic structures allows for less interaction with non-targeted macromolecules (including nucleic acids and proteins), have reduced polyanionic-related side effects, and will linearize in the presence of the targeted gene or RNA only. Additionally, due to cyclic structure, these CSOs can escape from endosomes due to a lack of interactions of 5′-end.

The oligonucleotides of the invention are isolated oligonucleotides. The term “isolated” means altered or removed from the natural state through human intervention. For example, an oligonucleotide naturally present in a living animal is not “isolated,” but a synthetic oligonucleotide, or an oligonucleotide partially or completely separated from the coexisting materials of its natural state is “isolated.” An isolated oligonucleotide can exist in substantially purified form, or can exist in a non-native environment such as, for example, a cell into which the oligonucleotide has been delivered. The oligonucleotides of the invention can comprise partially purified DNA and/or RNA, substantially pure DNA and/or RNA, synthetic DNA and/or RNA, or recombinantly produced DNA and/or RNA, as well as altered DNA and/or RNA that differs from naturally occurring DNA and/or RNA by the addition, deletion, substitution and/or alteration of one or more nucleotides. Such alterations can include addition of non-nucleotide material, such as to the end(s) of the oligonucleotide or to one or more internal nucleotides of the oligonucleotide, including modifications that make the oligonucleotide resistant to nuclease digestion.

The terms “microRNA,” “miRNA,” and “MiR” are interchangeable and refer to endogenous or artificial non-coding RNAs that can regulate gene expression. It is believed that miRNAs function via RNA interference. The design of such microRNAs is within the skill of ordinary artisans.

The terms “piRNA” and “Piwi-interacting RNA” are interchangeable and refer to a class of small RNAs involved in gene silencing. PiRNA molecules typically are between 26 and 31 nucleotides in length. The design of such PiRNAs is within the skill of ordinary artisans.

### Splitmer

In embodiments, the chemical makeup of the antisense oligonucleotide of the functional domain is as described in WO2020/191177, which is incorporated herein by reference in its entirety. In embodiments, the antisense oligonucleotide of the functional domain is a modified oligonucleotide comprising or consisting of an antisense oligonucleotide compound 17 to 25 nucleotides in length, wherein the antisense oligonucleotide compound comprises a 3′ domain and a 5′ domain, which is contiguous with the 3′ domain, wherein the 3′ domain begins at the terminal nucleotide at the 3′ end and is 10 to 12 nucleotides in length and each nucleotide comprises a deoxyribonucleotide and a phospodiester or phosphothioate internucleotide linkage or combinations thereof; and wherein the 5′ domain begins at the first nucleotide following the 3′ domain and continues to the terminal nucleotide at the 5′ end, wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone. Such antisense oligonucleotides are referred to as “splitmer”.

In embodiments, the antisense oligonucleotide is an oligonucleotide selected from Table 1.

In embodiments, the modified deoxyribonucleotides and/or modified ribonucleotides of the 5′ domain need not be consecutive.

In embodiments, the modified deoxyribonucleotides and/or modified ribonucleotides of the 5′ domain prevent RNase H cleavage in the 5′ domain.

In embodiments, the modified deoxyribonucleotide or modified ribonucleotide comprise a modified base, a modified sugar and/or modified backbone. In embodiments, the modified deoxyribonucleotide or modified ribonucleotide comprise a modified sugar and/or modified backbone.

In embodiments, at least half of the nucleotides of the 5′ domain comprise a modified deoxyribonucleotide or modified ribonucleotide comprising a modified sugar and/or backbone. In embodiments, at least half of the nucleotides of the 5′ domain comprise a modified ribonucleotide comprising a modified sugar and/or backbone.

When less than all of the nucleotides of the 5′ domain are modified, the modified and unmodified nucleotides are arranged so that no more than 2 unmodified nucleotides in the 5′ domain are next to each other. For example, the modified and unmodified nucleotides can alternate as singles or pairs within the 5′ domain.

In embodiments, all of the nucleotides of the 5′ domain comprise a modified deoxyribonucleotide or modified ribonucleotide comprising a modified sugar and/or backbone. In embodiments, all of the nucleotides of the 5′ domain comprise a modified ribonucleotide comprising a modified sugar and/or backbone.

In embodiments, the splitmer of the functional domain comprises 17 to 25 linked nucleotides having at least 12 contiguous nucleobases complementary to an equal length portion of a target RNA.

In embodiments, the modified ribonucleotides of the splitmer comprise 2′-substituted nucleotides are as described herein. In embodiments, the 2′-substituted nucleotides are selected from 2′ O-methyl ribonucleotides or (2′-OME) or 2′-methoxyethyl ribonucleosides (2′-MOE).

In some embodiments, the 3′ domain comprises nucleotides at positions 1, 2, 3, 4, 5, 6, 7, 8, 9, 10, 11, and 12 from the 3′ end. In some embodiments, the 3′ domain comprises nucleotides at positions 1, 2, 3, 4, 5, 6, 7, 8, 9, 10, and 11 from the 3′ end. In some embodiments, the 3′ domain comprises nucleotides at positions 1, 2, 3, 4, 5, 6, 7, 8, 9, and 10 from the 3′ end.

In some embodiments, when the 3′ domain of the antisense oligonucleotide is 12 nucleotides in length, the antisense oligonucleotides of the invention are represented by Formula (I):

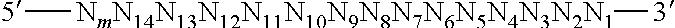

wherein
N is any nucleotide;
N₁₃ through Nₘ comprises the 5′ domain;
N₁ through N₁₂ comprises the 3′ domain; and
m is selected from 0, 1, 2, 3, 4, 5, 6, 7, 8, 9, 10, or 11.

In some embodiments, when the 3′ domain of the antisense oligonucleotide is 11 nucleotides in length, the antisense oligonucleotides of the invention are represented by Formula (Ia):

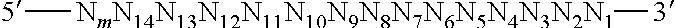

wherein
N is any nucleotide;
N₁₂ through Nₘ comprises the 5′ domain;
N₁ through N₁₁ comprises the 3′ domain; and
m is selected from 0, 1, 2, 3, 4, 5, 6, 7, 8, 9, 10, or 11.

In some embodiments, the antisense oligonucleotides of the invention are represented by Formula (Ib):

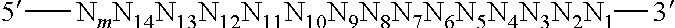

wherein
N is any nucleotide;
N₁₁ through Nₘ comprises the 5′ domain;
N₁ through N₁₀ comprises the 3′ domain; and
m is selected from 0, 1, 2, 3, 4, 5, 6, 7, 8, 9, 10, or 11.

In some embodiments, m is 0. In some embodiments, m is selected from 1, 2, 3, 4, 5, 6, or 7. In some embodiments, m is selected from 1, 2, 3, 4, 5, or 6. In some embodiments, m is selected from 1, 2, 3, 4, or 5. In some embodiments, m is selected from 1, 2, 3, or 4. In some embodiments, m is selected from 1, 2, or 3. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11.

In embodiments, the splitmer antisense oligonucleotide compound of the functional domain is 17 to 25 nucleotides in length comprising at least 12 contiguous nucleobases complementary to an equal length portion of a target RNA sequence, wherein the antisense oligonucleotide compound comprises a 3′ domain and a 5′ domain, which is contiguous with the 3′ domain, wherein the 3′ domain begins at the terminal nucleotide at the 3′ end and is 10 to 12 nucleotides in length and each nucleotide comprises a deoxyribonucleotide and a phospodiester or phosphothioate internucleotide linkage or combinations thereof; and wherein the 5′ domain begins at the first nucleotide following the 3′ domain and continues to the terminal nucleotide at the 5′ end, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone.

In embodiments, the antisense oligonucleotide is an oligonucleotide selected from Table 1.

In embodiments, the modified deoxyribonucleotides and/or modified ribonucleotides of the 5′ domain prevent RNase H cleavage in the 5′ domain. In embodiments, the modified deoxyribonucleotide or modified ribonucleotide comprise a modified base, a modified sugar and/or modified backbone. In embodiments, the modified deoxyribonucleotide or modified ribonucleotide comprise a modified sugar and/or modified backbone.

In embodiments, at least half of the nucleotides of the 5′ domain comprise a modified deoxyribonucleotide or modified ribonucleotide comprising a modified sugar and/or backbone. In embodiments, at least half of the nucleotides of the 5′ domain comprise a modified ribonucleotide comprising a modified sugar and/or backbone.

When less than all of the nucleotides of the 5′ domain are modified, the modified and unmodified nucleotides are arranged so that no more than 2 unmodified nucleotides in the 5′ domain are next to each other.

In embodiments, all of the nucleotides of the 5′ domain comprise a modified deoxyribonucleotide or modified ribonucleotide comprising a modified sugar and/or backbone. In embodiments, at least half of the nucleotides of the 5′ domain comprise a modified ribonucleotide comprising a modified sugar and/or backbone.

In embodiments, the 3′ domain is 12 nucleotides in length and comprises nucleotides at positions 1, 2, 3, 4, 5, 6, 7, 8, 9, 10, 11, and 12 from the 3′ end (position 1 is the 3′ end). In embodiments, the 3′ domain is 11 nucleotides in length and comprises nucleotides at positions 1, 2, 3, 4, 5, 6, 7, 8, 9, 10, and 11 from the 3′ end. In embodiments, the 3′ domain is 12 nucleotides in length and comprises nucleotides at positions 1, 2, 3, 4, 5, 6, 7, 8, 9, and 10 from the 3′ end.

In embodiments, the nucleotides of the 3′ domain comprise a natural nucleobase. In some embodiments, the nucleobases and sugars of the nucleotides of the 3′ domain of the antisense oligonucleotide according to the invention are unmodified. In this respect, the nucleobases and sugars of the nucleotides of the 3′ domain of the antisense oligonucleotide according to the invention are naturally occurring. Each of the nucleotides of the 3′ domain comprise a deoxyribonucleotide and a phosphodiester or phosphorothioate internucleotide linkage or combinations thereof. The nucleotides of the 3′-domain comprise natural deoxyribose sugar and phosphorothioate, phosphodiester or other phosphorus-based linkages or combinations thereof, which are known to activate RNase H.

In embodiments, at least one of the nucleotides of the 3′ domain comprises a modified nucleobase.

In embodiments, the nucleotides at the 9th or 10th positions from the 3′ end are not modified. In embodiments, the nucleotides at the 9th and 10th positions from the 3′ end are not modified. In embodiments, the nucleotide at the 11th position from the 3′ end is not modified. In embodiments, the nucleotides at the 9th, 10th, and 11th positions from the 3′ end are not modified. In embodiments, the nucleotide at the 12th position from the 3′ end is not modified. In embodiments, the nucleotides at the 9th, 10th, 11th, and 12th positions from the 3′ end are not modified.

In embodiments, the oligonucleotide comprises at least one phosphorothioate internucleotide linkage. In embodiments, at least half of the internucleotide linkages are phosphorothioate. In embodiments, all of the internucleotide linkages are phosphorothioate.

In embodiments, at least half of the internucleotide linkages are phosphodiester. In embodiments, all of the internucleotide linkages are phosphodiester.

In embodiments, the antisense oligonucleotide is single stranded.

As used here, the term “5′ domain” refers to the nucleotides beginning at the first nucleotide following the 3′ domain and goes to the 5′ end. The 5′ domain hybridizes to the target RNA but does not allow RNase H to excise the target RNA in this domain. The term “5′ domain” is generally 2 to 15 nucleotides in length and refers to the 11^{th} through the 25^{th} nucleotides (the 1^{st} nucleotide is the 3′ end), 12^{th} through the 25^{th} nucleotides, or 13^{th} through the 25^{th} nucleotides of the antisense oligonucleotide as measured from the 3′ end depending on the length of the 3′ domain.

For example, an antisense oligonucleotide compound that is 21 nucleotides in length may comprise a 3′ domain from position 1 to position 12 and a 5′ domain from position 13 to position 21. The designation of the modified nucleotide is position-specific, as opposed to nucleotide-specific.

The 5′ domain comprises nucleotides having non-RNase H activating modifications such as modified sugars and/or modified backbones, which do not activate RNase H. In some embodiments, the 5′ domain comprises nucleotides comprising a modified sugar. In some embodiments, the 5′ domain comprises nucleotides comprising a modified backbone. In some embodiments, the 5′ domain comprises nucleotides comprising both a modified sugar and modified backbone. In embodiments, the modified backbone is a non-phosphorus-based backbone.

This design of antisense allows for targeted RNA cleavage at the specific sites towards the 5′ end of 3′ domain. In embodiments, design of antisense allows for targeted RNA cleavage at the 9^{th}, 10^{th}, 11^{th}, or 12^{th} nucleotide positions from the 3′ end. In embodiments, design of antisense allows for targeted RNA cleavage at the 9^{th} nucleotide positions from the 3′ end. In embodiments, design of antisense allows for targeted RNA cleavage at the 10^{th} nucleotide positions from the 3′ end. In embodiments, design of antisense allows for targeted RNA cleavage at the 11^{th} nucleotide positions from the 3′ end. In embodiments, design of antisense allows for targeted RNA cleavage at the 12^{th} nucleotide positions from the 3′ end.

In any of these embodiments it is contemplated that at least half of the nucleotides of the 5′ domain comprise a backbone modification or substitution and/or a sugar modification or substitution. In some embodiments, the nucleotides at all positions within the 5′ domain comprises a backbone modification or substitution and/or a sugar modification or substitution. In one embodiment, the 5′ domain comprises at least two nucleotides comprising a modified backbone and/or sugar. In one embodiment, the 5′ domain comprises at least three nucleotides comprising a modified backbone and/or sugar. In one embodiment, the 5′ domain comprises at least four nucleotides comprising a modified backbone and/or sugar. In one embodiment, the 5′ domain comprises at least five nucleotides comprising a modified backbone and/or sugar. In one embodiment, the 5′ domain comprises at least six nucleotides comprising a modified backbone and/or sugar. In one embodiment, the 5′ domain comprises at least seven nucleotides comprising a modified backbone and/or sugar. In one embodiment, the 5′ domain comprises at least eight nucleotides comprising a modified backbone and/or sugar. In one embodiment, all of the nucleotides of the 5′ domain are nucleotides comprising a modified backbone and/or sugar.

It is specifically contemplated that embodiments discussed herein, in the context of a specific nucleotide and position, may be implemented with respect to a position relative to the 3′ end. For example, an antisense oligonucleotide with a modified nucleotide at position 13 refers to an antisense oligonucleotide having a modified nucleotide at position 13 from the 3′ end of the antisense oligonucleotide.

In embodiments, the antisense oligonucleotide of the functional domain is at least 90% complementary over its entire length to a portion of the target RNA.

Examples of splitmer antisense oligonucleotides useful as a functional domain within a cyclic structured oligonucleotide (CSO) according to the invention include, but are not limited to, the antisense oligonucleotides of Table 2. Internucleotide linkages are phosphorothioate linkages unless otherwise noted.

TABLE 2

| Cmp # | Target | Sequence (SEQ ID NO:) |
|---|---|---|
| 1 | ApoB-100 | 5′-G₁C₁C₁T₁C₁A₁G₁T₁CTGCTTCGCACC-3′ (SEQ ID NO: 1) |
| 2 | ApoB-100 | 5′-G₁C₁C₁T₁C₁A₁G₁T₁C₁TGCTTCGCACC-3′ (SEQ ID NO: 2) |
| 3 | ApoB-100 | 5′-G₃C₃C₃T₃C₃A₃G₃T₃CTGCTTCGCACC-3′ (SEQ ID NO: 3) |
| 4 | ApoB-100 | 5′-G₃C₃C₃T₃C₃A₃G₃T₃C₃TGCTTCGCACC-3′ (SEQ ID NO: 4) |
| 5 | TTR | 5′-T₁C₁T₁T₁G₁G₁T₁T₁ACATGAAATCCC-3′ (SEQ ID NO: 5) |
| 6 | TTR | 5′-T₁C₁T₁T₁G₁G₁T₁T₁A₁CATGAAATCCC-3′ (SEQ ID NO: 6) |
| 7 | TTR | 5′-T₃C₃T₃T₃G₃G₃T₃T₃ACATGAAATCCC-3′ (SEQ ID NO: 7) |
| 8 | TTR | 5′-T₃C₃T₃T₃G₃G₃T₃T₃A₃CATGAAATCCC-3′ (SEQ ID NO: 8) |
| 9 | APOC3 | 5′-A₁G₁C₁T₁T₁C₁T₁T₁GTCCAGCTTTAT-3′ (SEQ ID NO: 9) |
| 10 | APOC3 | 5′-A₁G₁C₁T₁T₁C₁T₁T₁G₁TCCAGCTTTAT-3′ (SEQ ID NO: 10) |
| 11 | APOC3 | 5′-A₃G₃C₃T₃T₃C₃T₃T₃GTCCAGCTTTAT-3′ (SEQ ID NO: 11) |
| 12 | APOC3 | 5′-A₃G₃C₃T₃T₃C₃T₃T₃G₃TCCAGCTTTAT-3′ (SEQ ID NO: 12) |
| 13 | PTP-1b | 5′-G₁C₁T₁C₁C₁T₁T₁C₁CACTGATCCTGC-3′ (SEQ ID NO: 13) |
| 14 | PTP-1b | 5′-G₁C₁T₁C₁C₁T₁T₁C₁C₁ACTGATCCTGC-3′ (SEQ ID NO: 14) |
| 15 | PTP-1b | 5′-G₃C₃T₃C₃C₃T₃T₃C₃CACTGATCCTGC-3′ (SEQ ID NO: 15) |
| 16 | PTP-1b | 5′-G₃C₃T₃C₃C₃T₃T₃C₃C₃ACTGATCCTGC-3′ (SEQ ID NO: 16) |
| 17 | PCSK9 | 5′-C₁C₁T₁T₁G₁G₁C₁C₁ACGCCGGCATCC-3′ (SEQ ID NO: 17) |
| 18 | PCSK9 | 5′-C₁C₁T₁T₁G₁G₁C₁C₁A₁CGCCGGCATCC-3′ (SEQ ID NO: 18) |
| 19 | PCSK9 | 5′-C₃C₃T₃T₃G₃G₃C₃C₃ACGCCGGCATCC-3′ (SEQ ID NO: 19) |
| 20 | PCSK9 | 5′-C₃C₃T₃T₃G₃G₃C₃C₃A₃CGCCGGCATCC-3′ (SEQ ID NO: 20) |
| 21 | STAT3 | 5′-C₁T₁A₁T₁TTGGATGTCAGC-3′ (SEQ ID NO: 21) |
| 22 | STAT3 | 5′-C₁T₁A₁T₁T₁TGGATGTCAGC-3′ (SEQ ID NO: 22) |
| 23 | STAT3 | 5′-C₃T₃A₃T₃TTGGATGTCAGC-3′ (SEQ ID NO: 23) |
| 24 | STAT3 | 5′-C₃T₃A₃T₃T₃TGGATGTCAGC-3′ (SEQ ID NO: 24) |
| 25 | ANGPTL3 | 5′-G₁G₁A₁C₁A₁T₁T₁G₁CCAGTAATCGCA-3′ (SEQ ID NO: 25) |
| 26 | ANGPTL3 | 5′-G₁G₁A₁C₁A₁T₁T₁G₁C₁CAGTAATCGCA-3′ (SEQ ID NO: 26) |
| 27 | ANGPTL3 | 5′-G₃G₃A₃C₃A₃T₃T₃G₃CCAGTAATCGCA-3′ (SEQ ID NO: 27) |
| 28 | ANGPTL3 | 5′-G₃G₃A₃C₃A₃T₃T₃G₃C₃CAGTAATCGCA-3′ (SEQ ID NO: 28) |
| 29 | Kallikrein B1 | 5′-T₁G₁C₁A₁A₁G₁T₁C₁TCTTGGCAAACA-3′ (SEQ ID NO: 29) |
| 30 | Kallikrein B1 | 5′-T₁G₁C₁A₁A₁G₁T₁C₁T₁CTTGGCAAACA-3′ (SEQ ID NO: 30) |
| 31 | Kallikrein B1 | 5′-T₃G₃C₃A₃A₃G₃T₃C₃TCTTGGCAAACA-3′ (SEQ ID NO: 31) |
| 32 | Kallikrein B1 | 5′-T₃G₃C₃A₃A₃G₃T₃C₃T₃CTTGGCAAACA-3′ (SEQ ID NO: 32) |
| 33 | DGAT2 | 5′-T₁G₁C₁C₁A₁T₁T₁T₁AATGAGCTTCAC-3′ (SEQ ID NO: 33) |
| 34 | DGAT2 | 5′-T₁G₁C₁C₁A₁T₁T₁T₁A₁ATGAGCTTCAC-3′ (SEQ ID NO: 34) |
| 35 | DGAT2 | 5′-T₃G₃C₃C₃A₃T₃T₃T₃AATGAGCTTCAC-3′ (SEQ ID NO: 35) |
| 36 | DGAT2 | 5′-T₃G₃C₃C₃A₃T₃T₃T₃A₃ATGAGCTTCAC-3′ (SEQ ID NO: 36) |
| 37 | SOD1 | 5′-C₁A₁G₁G₁A₁T₁A₁C₁ATTTCTACAGCT-3′ (SEQ ID NO: 37) |
| 38 | SOD1 | 5′-C₁A₁G₁G₁A₁T₁A₁CATTTCTACAGCT-3′ (SEQ ID NO: 38) |
| 39 | SOD1 | 5′-C₃A₃G₃G₃A₃T₃A₃C₃ATTTCTACAGCT-3′ (SEQ ID NO: 39) |
| 40 | SOD1 | 5′-C₃A₃G₃G₃A₃T₃A₃CATTTCTACAGCT-3′ (SEQ ID NO: 40) |
| 41 | SOD1 | 5′-C₃A₄G₃G₄A₃T₄A₃C₃ATTTCTACAGCT-3′ (SEQ ID NO: 41) |
| 42 | SOD1 | 5′-C₃A₄G₃G₄A₃T₄A₃CATTTCTACAGCT-3′ (SEQ ID NO: 42) |
| 43 | SOD1 | 5′-C₁A₁G₁G₁A₁T₁A₁C₁ATTTCTACAGCT-3′ (SEQ ID NO: 43) |
| 44 | SOD1 | 5′-C₁A₁G₁G₁A₁T₁A₁CATTTCTACAGCT-3′ (SEQ ID NO: 44) |
| 45 | SOD1 | 5′-C₃A₃G₃G₃A₃T₃A₃C₃ATTTCTACAGCT-3′ (SEQ ID NO: 45) |
| 46 | SOD1 | 5′-C₃A₃G₃G₃A₃T₃A₃CATTTCTACAGCT-3′ (SEQ ID NO: 46) |
| 47 | SOD1 | 5′-C₃A₄G₃G₄A₃T₄A₃C₃ATTTCTACAGCT-3′ (SEQ ID NO: 47) |
| 48 | SOD1 | 5′-C₃A₄G₃G₄A₃T₄A₃CATTTCTACAGCT-3′ (SEQ ID NO: 48) |
| 49 | FUS | 5′-C₁C₁A₁U1A₁C₁T₁T₁TCCACTGUAACG-3′ (SEQ ID NO: 49) |
| 50 | FUS | 5′-C₃C₃A₃U3A₃C₃T₃T₃TCCACTGUAACG-3′ (SEQ ID NO: 50) |
| 51 | FUS | 5′-C₃C₃A₃U3A₃C₃T₃T₃T₃CCACTGUAACG-3′ (SEQ ID NO: 51) |
| 52 | APO(a) | 5′-T₁G₁C₁T₁C₁C₁G₁T₁T₁GGTGCTTGTTC-3′ (SEQ ID NO: 52) |
| 53 | APO(a) | 5′-T₁G₁C₁T₁C₁C₁G₁T₁TGGTGCTTGTTC-3′ (SEQ ID NO: 53) |
| 54 | APO(a) | 5′-T₃G₃C₃T₃C₃C₃G₃T₃T₃GGTGCTTGTTC-3′ (SEQ ID NO: 54) |
| 55 | APO(a) | 5′-T₃G₃C₃T₃C₃C₃G₃T₃TGGTGCTTGTTC-3′ (SEQ ID NO: 55) |
| 56 | HTT | 5′-C₁U₁C₁A₁G₁T₁A₁A₁C₁ATTGACACCAC-3′ (SEQ ID NO: 56) |
| 57 | HTT | 5′-C₁U₁C₁A₁G₁T₁A₁A₁C₁A₁TTGACACCAC-3′ (SEQ ID NO: 57) |
| 58 | HTT | 5′-C₃U₃C₃A₃G₃T₃A₃A₃C₃A₃TTGACACCAC-3′ (SEQ ID NO: 58) |
| 59 | HTT | 5′-C₃U₃C₃A₃G₃T₃A₃A₃C₃ATTGACACCAC-3′ (SEQ ID NO: 59) |
| 60 | HBV | 5′-G₁C₁A₁G₁A₁G₁G₁T₁G₁AAGCGAAGTGC-3′ (SEQ ID NO: 60) |
| 61 | HBV | 5′-G₁C₁A₁G₁A₁G₁G₁T₁GAAGCGAAGTGC-3′ (SEQ ID NO: 61) |
| 62 | HBV | 5′-G₃C₃A₃G₃A₃G₃G₃T₃G₃AAGCGAAGTGC-3′ (SEQ ID NO: 62) |
| 63 | HBV | 5′-G₃C₃A₃G₃A₃G₃G₃T₃GAAGCGAAGTGC-3′ (SEQ ID NO: 63) |
| 64 | Angiotensinogen | 5′-G₁G₁G₁C₁C₁TGTTTAGTCGC-3′ (SEQ ID NO: 64) |
| 65 | Angiotensinogen | 5′-G₁G₁G₁C₁CTGTTTAGTCGC-3′ (SEQ ID NO: 65) |
| 66 | Angiotensinogen | 5′-G₃G₃G₃C₃C₃TGTTTAGTCGC-3′ (SEQ ID NO: 66) |
| 67 | Angiotensinogen | 5′-G₃G₃G₃C₃CTGTTTAGTCGC-3′ (SEQ ID NO: 67) |
| 68 | Angiotensinogen | 5′-C₁A₁C₁A₁A₁A₁C₁A₁A₁GCTGGTCGGTT-3′ (SEQ ID NO: 68) |
| 69 | Angiotensinogen | 5′-C₁A₁C₁A₁A₁A₁C₁A₁AGCTGGTCGGTT-3′ (SEQ ID NO: 69) |
| 70 | Angiotensinogen | 5′-C₃A₃C₃A₃A₃A₃C₃A₃A₃GCTGGTCGGTT-3′ (SEQ ID NO: 70) |
| 71 | Angiotensinogen | 5′-C₃A₃C₃A₃A₃A₃C₃A₃AGCTGGTCGGTT-3′ (SEQ ID NO: 71) |
| 72 | Factor XI | 5′-A₁C₁G₁G₁C₁A₁T₁T₁GGTGCACAGTTT-3′ (SEQ ID NO: 72) |
| 73 | Factor XI | 5′-A₁C₁G₁G₁C₁A₁T₁T₁G₁GTGCACAGTTT-3′ (SEQ ID NO: 73) |
| 74 | Factor XI | 5′-A₃C₃G₃G₃C₃A₃T₃T₃GGTG₃CACAGTTT-3′ (SEQ ID NO: 74) |
| 75 | Factor XI | 5′-A₃C₃G₃G₃C₃A₃T₃T₃G₃GTGCACAGTTT-3′ (SEQ ID NO: 75) |
| 76 | SMN2 | 5′-T₁C₁A₁C₁T₁T₁T₁C₁A₁T₁A₁A₁T₁G₁CTGG-3′ (SEQ ID NO: 76) |
| 77 | SMN2 | 5′-T₃C₃A₃C₃T₃T₃T₃C₃A₃T₃A₃A₃T₃G₃CTGG-3′ (SEQ ID NO: 77) |
| 78 | TMPRSS6 | 5′-C₁T₁T₁T₁A₁T₁T₁C₁CAAAGGGCAGCT-3′ (SEQ ID NO: 78) |
| 79 | TMPRSS6 | 5′-C₁T₁T₁T₁A₁T₁T₁C₁CAAAGGGCAGCT-3′ (SEQ ID NO: 79) |
| 80 | TMPRSS6 | 5′-C₃T₃T₃T₃A₃T₃T₃C₃CAAAGGGCAGCT-3′ (SEQ ID NO: 80) |
| 81 | TMPRSS6 | 5′-C₃T₃T₃T₃A₃T₃T₃C₃C₃AAAGGGCAGCT-3′ (SEQ ID NO: 81) |
| 82 | MAPT | 5′-C₁C₁G₁T₁T₁T₁T₁CTTACCACCCT-3′ (SEQ ID NO: 82) |
| 83 | MAPT | 5′-C₁C₁G₁T₁T₁T₁TCTTACCACCCT-3′ (SEQ ID NO: 83) |
| 84 | MAPT | 5′-C₃C₃G₃T₃T₃T₃T₃CTTACCACCCT-3′ (SEQ ID NO: 84) |
| 85 | MAPT | 5′-C₃C₃G₃T₃T₃T₃TCTTACCACCCT-3′ (SEQ ID NO: 85) |
| 86 | PTP1B (PTPN1) | 5′-A₁T₁G₁G₁T₁T₁T₁A₁TTCCATGGCCA-3′ (SEQ ID NO: 86) |
| 87 | PTP1B (PTPN1) | 5′-A₁T₁G₁G₁T₁T₁T₁ATTCCATGGCCA-3′ (SEQ ID NO: 87) |
| 88 | PTP1B (PTPN1) | 5′-A₃T₃G₃G₃T₃T₃T₃A₃TTCCATGGCCA-3′ (SEQ ID NO: 88) |
| 89 | PTP1B (PTPN1) | 5′-A₃T₃G₃G₃T₃T₃T₃ATTCCATGGCCA-3′ (SEQ ID NO: 89) |
| 90 | HTT | 5′-C₁U₁U₁U₁C₁A₁G₁A₁C₁A₁CGGGAACACGG-3′ (SEQ ID NO: 90) |
| 91 | HTT | 5′-C₁U₁U₁U₁C₁A₁G₁A₁C₁ACGGGAACACGG-3′ (SEQ ID NO: 91) |
| 92 | HTT | 5′-C₃U₃U₃U₃C₃A₃G₃A₃C₃A₃CGGGAACACGG-3′ (SEQ ID NO: 92) |
| 93 | HTT | 5′-C₃U₃U₃U₃C₃A₃G₃A₃C₃ACGGGAACACGG-3′ (SEQ ID NO: 93) |
| 94 | ATXN2 | 5′-C₁T₁T₁C₁A₁C₁A₁T₁T₁TCGATCCAACA-3′ (SEQ ID NO: 94) |
| 95 | ATXN2 | 5′-C₁T₁T₁C₁A₁C₁A₁T₁TTCGATCCAACA-3′ (SEQ ID NO: 95) |
| 96 | ATXN2 | 5′-C₃T₃T₃C₃A₃C₃A₃T₃T₃TCGATCCAACA-3′ (SEQ ID NO: 96) |
| 97 | ATXN2 | 5′-C₃T₃T₃C₃A₃C₃A₃T₃TTCGATCCAACA-3′ (SEQ ID NO: 97) |
| 98 | SCNIA | 5′-A₁G₁T₁T₁G₁G₁A₁G₁C₁A₁A₁G₁A₁T₁TATC-3′ (SEQ ID NO: 98) |
| 99 | SCNIA | 5′-A₃G₃T₃T₃G₃G₃A₃G₃C₃A₃A₃G₃A₃T₃TATC-3′ (SEQ ID NO: 99) |
| 100 | DMD | 5′-C₁A₁A₁T₁G₁C₁C₁A₁T₁C₁C₁T₁G₁G₁A₁G₁T₁T₁CCTG-3′ (SEQ ID NO: 100) |
| 101 | DMD | 5′-C₃A₃A₃T₃G₃C₃C₃A₃T₃C₃C₃T₃G₃G₃A₃G₃T₃T₃CCTG-3′ (SEQ ID NO: 101) |
| 102 | DMD | 5′-G₁T₁T₁G₁C₁C₁T₁C₁C₁G₁G₁T₁T₁C₁T₁G₁A₁A₁G₁G₁T₁GTTC-3′ |
| | | (SEQ ID NO: 102) |
| 103 | DMD | 5′-G₃T₃T₃G₃C₃C₃T₃C₃C₃G₃G₃T₃T₃C₃T₃G₃A₃A₃G₃G₃T₃GTTC-3′ |
| | | (SEQ ID NO: 103) |
| 104 | DMD | 5′-C₁T₁C₁C₁A₁A₁C₁A₁T₁C₁A₁A₁G₁G₁A₁A₁G₁A₁T₁G₁G₁C₁A₁T₁T₁T₁CTAG-3′ |
| | | (SEQ ID NO: 104) |
| 105 | DMD | 5′-C₃ T₃C₃C₃A₃A₃C₃A₃T₃C₃A₃A₃G₃G₃A₃A₃G₃A₃T₃G₃G₃C₃A₃T₃T₃T₃CTAG-3′ |
| | | (SEQ ID NO: 105) |
| 154 | FUS | 5′-C₁C₁A₁U₁A₁C₁T₁T₁T₁CCACTGUAACG-3′ (SEQ ID NO: 154) |
| 307 | HTT | 5′-C₃T₃C₃A₃G₃T₃A₃A₃C₃ATTGACACCAC-3′ (SEQ ID NO: 307) |
| 308 | HTT | 5′-C₃T₃C₃A₃G₃T₃A₃A₃CATTGACACCAC-3′ (SEQ ID NO: 308) |
| 309 | HTT | 5′-C₄T₃C₄A₃G₄T₃A₄A₃C₃ATTGACACCAC-3′ (SEQ ID NO: 309) |
| 310 | HTT | 5′-C₄T₃C₄A₃G₄T₃A₄A₃C₄atTGACACCAC-3′ (SEQ ID NO: 310) |
| 311 | HTT | 5′-C₄T₃C₄A₃G₄T₃A₄A₃C₃attGACACCAC-3′ (SEQ ID NO: 311) |
| 312 | HTT | 5′-C₄T₃C₄A₃G₄T₃A₄A₃C₃AttgACACCAC-3′ (SEQ ID NO: 312) |
| 313 | HTT | 5′-C₄T₃C₄A₃G₄T₃A₄A₃C₃atTGACACCAC-3′ (SEQ ID NO: 313) |
| 314 | HTT | 5′-C₄T₃C₄A₃G₄T₃A₄A₃C₃AttGACACCAC-3′ (SEQ ID NO: 314) |
| 315 | MAPT | 5′-C₃C₃G₃T₃T₃T₃T₃CTTACCACCCT-3 (SEQ ID NO: 315) |
| 316 | MAPT | 5′-C₃C₃G₄T₄T₄T₄T₃CTTACCACCCT-3′ (SEQ ID NO: 316) |
| 317 | SOD1 | 5′-C₄A₄G₃G₃A₃T₃A₃C₃ATTTCTACAGCT-3′ (SEQ ID NO: 317) |
| 318 | SOD1 | 5′-C₄A₄G₄G₃A₃T₃A₃C₃ATTTCTACAGCT-3′ (SEQ ID NO: 318) |
| 319 | SOD1 | 5′-C₄A₄G₄G₄A₃T₃A₃C₃ATTTCTACAGCT-3′ (SEQ ID NO: 319) |
| 320 | SOD1 | 5′-C₃A₄G₃GAA₃TACATTTCTACJA₃G₄C₃T₃A₂C₂A₂G₂C₂T₂-3′ (SEQ ID NO: |
| | | 320) |
| 321 | SOD1 | 5′-C₄A₄G₃G₃A₃T₃A₃C₃ATTTCTACAGCTA₂C₂A₂G₂C₂T₂-3′ (SEQ ID NO: 321) |
| 322 | SOD1 | 5′-C₄A₄G₄G₃A₃T₃A₃C₃ATTTCTACAGCTA₂C₂A₂G₂C₂T₂-3′ (SEQ ID NO: 322) |
| 323 | SOD1 | 5′-C₃A₃G₃G₃A₃T₃A₃C₃A₃TTTCTACAGCTA₂G₂C₂A₂G₂G₂-3′ (SEQ ID NO: |
| | | 323) |
| 324 | SOD1 | 5′-C₄A₄G₄G₃A₃T₃A₃C₃A₃TTTCTACAGCTA₂G₂C₂A₂G₂G₂-3′ (SEQ ID NO: |
| | | 324) |
| 325 | SOD1 | 5′-C₃A₃G₃G₃A₃TACATTTCTAC₃A₃G₃C₃T₃-3′ (SEQ ID NO: 325) |
| 326 | SOD1 | 5′-C₃A₄G₃G₄A₃TACATTTCTAC₄A₃G₄C₃T₃T₂A₂G₂C₂A₂G₂G₂-3′ (SEQ ID NO: |
| | | 326) |
| 327 | APOC3 | 5′-G₃C₃T₃T₃C₃T₃T₃G₃T₃CCAGCTTTATT-3′ (SEQ ID NO: 327) |
| 328 | APOC3 | 5′-G₄C₄T₄T₃C₃T₃T₃G₃T₃CCAGCTTTATT-3′ (SEQ ID NO: 328) |
| 329 | APOC3 | 5′-G₃C₃T₃T₃CT₃T₃G₃T₃CCAGCTTTATT-3′ (SEQ ID NO: 329) |
| 330 | APOC3 | 5′-G₃C₃T₃T₃C₃T₃T₃G₃T₃CCAGCTTTATTG₂G₂G₂A₂G₂-3′ (SEQ ID NO: 330) |
| 331 | APOC3 | 5′-G₄C₄T₄T₃C₃T₃T₃G₃T₃CCAGCTTTATTG₂G₂G₂A₂G₂-3′ (SEQ ID NO: 331) |
| 379 | SOD1 | 5′-C₃A₃G₃G₃A₃TACATTTCTAC₄A₃G₄C₃T₃T₂A₂G₂C₂A₂G₂G₂-3′ (SEQ ID NO: |
| | | 379) |

GCAT-DNA phosphorothioate linkage; G₁/C₁/A₁/T₁/U₁₋₂′OME phosphorothioate linkage; A₂/T₂/C₂/G₂-DNA phosphodiester linkage; G₃/C₃/A₃/T₃/U₃₋₂′MOE phosphorothioate linkage; G₄/C₄/A₄/T₄/U₄-2′MOE phosphodiester linkage; lowercase g/c/a/t—is phosphodiester DNA

Examples of cyclizing domains useful in the cyclic oligonucleotides described herein include, but are not limited to, the cyclizing domains of Table 3. Table 3 also provides functional domain (compound #(s) (Cmp #(s)) that (a portion of which) is complementary to the cyclizing domain. The internucleotide linkages for the oligonucleotides in Table 3 are phosphorothioate internucleotide linkages; phosphodiester internucleotide linkages, or combinations thereof.

TABLE 3

| | | functional domain |
|---|---|---|
| SEQ ID NO: | Sequence | (Cmp #s) |
| SEQ ID NO: 106 | 5′-GGTGCG-3′ | Compounds 1-4 |
| SEQ ID NO: 107 | 5′-GGGATT-3′ | Compounds 5-8 |
| SEQ ID NO: 108 | 5′-ATAAAG-3′ | Compounds 9-12 |
| SEQ ID NO: 109 | 5′-GCAGGA-3′ | Compounds 13-16 |
| SEQ ID NO: 110 | 5′-GGA₂TGC-3′ | Compounds 17-20 |
| SEQ ID NO: 111 | 5′-GCTGAC-3′ | Compounds 21-24 |
| SEQ ID NO: 112 | 5′-TGCGAT-3′ | Compounds 25-28 |
| SEQ ID NO: 113 | 5′-TGTTTG-3′ | Compounds 29-32 |
| SEQ ID NO: 114 | 5′-GTGAAG-3′ | Compounds 33-36 |
| SEQ ID NO: 115 | 5′-AGCTGT-3′ | Compounds 37-48 |
| SEQ ID NO: 116 | 5′-CGTTAC-3′ | Compounds 49-51 |
| | | and 154 |
| SEQ ID NO: 117 | 5′-CGTTAC-3′ | Compounds 49-51 |
| | | and 154 |
| SEQ ID NO: 118 | 5′-CGTTAC-3′ | Compounds 49-51 |
| | | and 154 |
| SEQ ID NO: 119 | 5′-GAACAA-3′ | Compounds 52-55 |
| SEQ ID NO: 120 | 5′-GAACA-3′ | Compounds 52-55 |
| SEQ ID NO: 121 | 5′-GTGGTG-3′ | Compounds 56-59 |
| SEQ ID NO: 122 | 5′-GTGGT-3′ | Compounds 56-59 |
| SEQ ID NO: 123 | 5′-GCACTT-3′ | Compounds 60-63 |
| SEQ ID NO: 124 | 5′-GCACT-3′ | Compounds 60-63 |
| SEQ ID NO: 125 | 5′-GCGACT-3′ | Compounds 64-67 |
| SEQ ID NO: 126 | 5′-GCGAC-3′ | Compounds 64-67 |
| SEQ ID NO: 127 | 5′-AACCGA-3′ | Compounds 68-71 |
| SEQ ID NO: 128 | 5′-AACCG-3′ | Compounds 68-71 |
| SEQ ID NO: 129 | 5′-AAACTG-3′ | Compounds 72-75 |
| SEQ ID NO: 130 | 5′-AAACT-3′ | Compounds 72-75 |
| SEQ ID NO: 130 | 5′-CCAGCA-3′ | Compounds 76-77 |
| SEQ ID NO: 130 | 5′-CCAGC-3′ | Compounds 76-77 |
| SEQ ID NO: 130 | 5′-CCAGCA-3′ | Compounds 76-77 |
| SEQ ID NO: 131 | 5′-AGCTGC-3′ | Compounds 79-81 |
| SEQ ID NO: 132 | 5′-AGCTG-3′ | Compounds 79-81 |
| SEQ ID NO: 133 | 5′-AGCTGC-3′ | Compounds 79-81 |
| SEQ ID NO: 134 | 5′-AGCTGC-3′ | Compounds 79-81 |
| SEQ ID NO: 135 | 5′-AGGGTG-3′ | Compounds 82-85 |
| SEQ ID NO: 136 | 5′-AGGGT-3′ | Compounds 82-85 |
| SEQ ID NO: 137 | 5′-TGGCCA-3′ | Compounds 86-89 |
| SEQ ID NO: 138 | 5′-TGGCC-3′ | Compounds 86-89 |
| SEQ ID NO: 139 | 5′-CCGTGT-3′ | Compounds 90-93 |
| SEQ ID NO: 140 | 5′-CCGTG-3′ | Compounds 90-93 |
| SEQ ID NO: 141 | 5′-CCGTGT-3′ | Compounds 90-93 |
| SEQ ID NO: 142 | 5′-CCGTGT-3′ | Compounds 90-93 |
| SEQ ID NO: 143 | 5′-TGTTGG-3′ | Compounds 94-97 |
| SEQ ID NO: 144 | 5′-TGTTG-3′ | Compounds 94-97 |
| SEQ ID NO: 145 | 5′-GATAATC-3′ | Compounds 98-99 |
| SEQ ID NO: 146 | 5′-GATAAT-3′ | Compounds 98-99 |
| SEQ ID NO: 147 | 5′-GATAA-3′ | Compounds 98-99 |
| SEQ ID NO: 148 | 5′-CAGGAA-3′ | Compounds 100-101 |
| SEQ ID NO: 149 | 5′-CAGGA-3′ | Compounds 100-101 |
| SEQ ID NO: 150 | 5-′GAACAC-3′ | Compounds 102-103 |
| SEQ ID NO: 151 | 5-′GAACA-3′ | Compounds 102-103 |
| SEQ ID NO: 152 | 5′-CTAGAA-3′ | Compounds 104-105 |
| SEQ ID NO: 153 | 5′-CTAGA-3′ | Compounds 104-105 |
| SEQ ID NO: 332 | 5′-AATAAA-3′ | Compounds 368, 370, |
| | | 372-374 |
| SEQ ID NO: 333 | 5-AATAAAG-3′ | Compounds 369 and |
| | | 371 |
| SEQ ID NO: 334 | 5′-AGCTGT-3′ | Compounds 356-363 |
| SEQ ID NO: 335 | 5′-CCTGCT--3 | Compounds 364-367 |
| SEQ ID NO: 336 | 5′-CTCCCA-3′ | Compounds 375-378 |

Examples of cyclic structured oligonucleotides (CSO) useful for gene silencing include, but are not limited to, the cyclic structured oligonucleotides of Table 4. All internucleotide linkages are phosphorothioate linkages unless otherwise noted. The SEQ ID NOs for the 5 cyclizing domain and functional domain of the compounds shown below are found in Tables 2 and 3 above.

TABLE 4

| Cmp # | Sequence | Target |
|---|---|---|
| 155 | gcgtgg-5′-5′-G₁C₁C₁T₁C₁A₁G₁T₁CTGCTTCGCACC-3′ | ApoB-100 |
| 156 | gcgtgg-5′-5′-G₁C₁C₁T₁C₁A₁G₁T₁C₁TGCTTCGCACC-3′ | ApoB-100 |
| 157 | gcgtgg-5′-5′-G₃C₃C₃T₃C₃A₃G₃T₃C₃TGCTTCGCACC-3′ | ApoB-100 |
| 158 | gcgtgg-5′-5′-G₃C₃C₃T₃C₃A₃G₃T₃CTGCTTCGCACC-3′ | ApoB-100 |
| 159 | ttaggg-5′-5′-T₁C₁T₁T₁G₁G₁T₁T₁AICATGAAATCCC-3′ | TTR |
| 160 | ttaggg-5′-5′-T₁C₁T₁T₁G₁G₁T₁T₁ACATGAAATCCC-3′ | TTR |
| 161 | ttaggg-5′-5′-T₃C₃T₃T₃G₃G₃T₃T₃A₃CATGAAATCCC-3′ | TTR |
| 162 | ttaggg-5′-5′-T₃C₃T₃T₃G₃G₃T₃T₃ACATGAAATCCC-3′ | TTR |
| 163 | gaaata-5′-5′-A₁G₁C₁T₁T₁C₁T₁T₁GTCCAGCTTTAT-3′ | APOC3 |
| 164 | gaaata-5′-5′-A₁G₁C₁T₁T₁C₁T₁T₁GTCCAGCTTTAT-3′ | APOC3 |
| 165 | gaaata-5′-5′-A₃G₃C₃T₃T₃C₃T₃T₃GTCCAGCTTTAT-3′ | APOC3 |
| 166 | gaaata-5′-5′-A₃G₃C₃T₃T₃C₃T₃T₃GTCCAGCTTTAT-3′ | APOC3 |
| 167 | aggacg-5′-5′-G₁C₁T₁C₁C₁T₁T₁C₁C₁ACTGATCCTGC-3′ | PTP-1b |
| 168 | aggacg-5′-5′-G₁C₁T₁C₁C₁T₁T₁C₁CACTGATCCTGC-3′ | PTP-1b |
| 169 | aggacg-5′-5′-G₃C₃T₃C₃C₃T₃T₃C₃C₃ACTGATCCTGC-3′ | PTP-1b |
| 170 | aggacg-5′-5′-G₃C₃T₃C₃C₃T₃T₃C₃CACTGATCCTGC-3′ | PTP-1b |
| 171 | cgtagg-5′-5′-C₁C₁T₁T₁G₁G₁C₁C₁A₁CGCCGGCATCC-3′ | PCSK9 |
| 172 | cgtagg-5′-5′-C₁C₁T₁T₁G₁G₁C₁C₁ACGCCGGCATCC-3′ | PCSK9 |
| 173 | cgtagg-5′-5′-C₃C₃T₃T₃G₃G₃C₃C₃ACGCCGGCATCC-3′ | PCSK9 |
| 174 | cgtagg-5′-5′-C₃C₃T₃T₃G₃G₃C₃C₃A₃CGCCGGCATCC-3′ | PCSK9 |
| 175 | cagtcg-5′-5′-C₁T₁A₁T₁T₁TGGATGTCAGC-3′ | STAT3 |
| 176 | cagtcg-5′-5′-C₁T₁A₁T₁TTGGATGTCAGC-3′ | STAT3 |
| 177 | cagtcg-5′-5′-C₃T₃A₃T₃TTGGATGTCAGC-3′ | STAT3 |
| 178 | cagtcg-5′-5′-C₃T₃A₃T₃T₃TGGATGTCAGC-3′ | STAT3 |
| 179 | tagcgt-5′-5′-G₁G₁A₁C₁A₁T₁T₁G₁CCAGTAATCGCA-3′ | ANGPTL3 |
| 180 | tagcgt-5′-5′-G₁G₁A₁C₁A₁T₁T₁G₁C₁CAGTAATCGCA-3′ | ANGPTL3 |
| 181 | tagcgt-5′-5′-G₃G₃A₃C₃A₃T₃T₃G₃CCAGTAATCGCA-3′ | ANGPTL3 |
| 182 | tagcgt-5′-5′-G₃G₃A₃C₃A₃T₃T₃G₃C₃CAGTAATCGCA-3′ | ANGPTL3 |
| 183 | gtttgt-5′-5′-T₁G₁C₁A₁A₁G₁T₁C₁TCTTGGCAAACA-3′ | Kallikrein B1 |
| 184 | gtttgt-5′-5′-T₁G₁C₁A₁A₁G₁T₁C₁T₁CTTGGCAAACA-3′ | Kallikrein B1 |
| 185 | gtttgt-5′-5′-T₃G₃C₃A₃A₃G₃T₃C₃TCTTGGCAAACA-3′ | Kallikrein B1 |
| 186 | gtttgt-5′-5′-T₃G₃C₃A₃A₃G₃T₃C₃T₃CTTGGCAAACA-3′ | Kallikrein B1 |
| 187 | gaagtg-5-5′-T₁G₁C₁C₁A₁T₁T₁T₁AATGAGCTTCAC-3′ | DGAT₂ |
| 188 | gaagtg-5-5′-T₁G₁C₁C₁A₁T₁T₁T₁A₁ATGAGCTTCAC-3′ | DGAT₂ |
| 189 | gaagtg-5′-5′-T₃G₃C₃C₃A₃T₃T₃T₃AATGAGCTTCAC-3′ | DGAT₂ |
| 190 | gaagtg-5′-5′-T₃G₃C₃C₃A₃T₃T₃T₃A₃ATGAGCTTCAC-3′ | DGAT₂ |
| 191 | acgacc-5′-5′-T₁C₁A₁C₁T₁T₁T₁C₁AIT₁A₁A₁T₁G₁CTGG-3′ | SMN2 |
| 192 | cgacc-5′-5′-T₁C₁A₁C₁T₁T₁T₁C₁AIT₁A₁A₁T₁G₁CTGG-3′ | SMN2 |
| 193 | acgacc-5′-5′-T₃C₃A₃C₃T₃T₃T₃C₃A₃T₃A₃A₃T₃G₃CTGG-3′ | SMN2 |
| 194 | cgacc-5′-5′-T₃C₃A₃C₃T₃T₃T₃C₃A₃T₃A₃A₃T₃G₃CTGG-3′ | SMN2 |
| 195 | acgacc-5′-5′-T₁C₁A₁C₁T₁T₁T₁C₁A₁T₁A₁A₁T₁G₁CTGG-3′ | SMN2 |
| 196 | acgacc-5′-5′-T₃C₃A₃C₃T₃T₃T₃C₃A₃T₃A₃A₃T₃G₃CTGG-3′ | SMN2 |
| 197 | 3′-GCAAUGTCACCT₅T₅T₅C₅A₅U₅A₅C₅C₅-5′-5′-cgttac | FUS |
| 198 | 3′-GCAAUGTCACCTT₅T₅C₅A₅U₅A₅C₅C₅-5′-5′-cgttac | FUS |
| 199 | 3′-GCAAUGTCACCT₅T₅T₅C₅A₅U₅A₅CC₅-5′-5′-c₅g₅t₅t₅a₅c₅ | FUS |
| 200 | 3′-GCAAUGTCACCTT₅T₅C₅A₅U₅A₅C₅C₅-5′-5′-c₅g₅t₅t₅a₅c₅ | FUS |
| 201 | aacaag-5′-5′-T₅G₅C₅T₅C₅C₅G₅T₅T₅GGTGCTTGTTC-3′ | APO(a) |
| 202 | aacaag-5′-5′-T₅G₅C₅T₅C₅C₅G₅T₅TGGTGCTTGTTC-3′ | APO(a) |
| 203 | acaag-5′-5′-T₅G₅C₅T₅C₅C₅G₅T₅T₅GGTGCTTGTTC-3′ | APO(a) |
| 204 | acaag-5′-5′-T₅G₅C₅T₅C₅C₅G₅T₅TGGTGCTTGTTC-3′ | APO(a) |
| 205 | gtggtg-5′-5′-C₅U₅C₅A₅GST₅A₅A₅C₅ATTGACACCAC | HTT |
| 206 | gtggtg-5′-5′-C₅U₅C₅A₅G₅T₅A₅A₅CATTGACACCAC | HTT |
| 207 | tggtg-5′-5′-C₅U₅C₅A₅G₅T₅A₅A₅C₅ATTGACACCAC | HTT |
| 208 | tggtg-5′-5′-C₅U₅C₅A₅GT₅A₅A₅CATTGACACCAC | HTT |
| 209 | ttcacg-5′-5′-G₅C₅A₅G₅A₅G₅G₅T₅G₅AAGCGAAGTGC-3′ | HBV |
| 210 | ttcacg-5′-5′-G₅C₅A₅G₅A₅G₅G₅T₅GAAGCGAAGTGC-3′ | HBV |
| 211 | tcacg-5′-5′-G₅C₅A₅G₅A₅G₅G₅T₅G₅AAGCGAAGTGC-3′ | HBV |
| 212 | tcacg-5′-5′-G₅C₅A₅G₅A₅G₅G₅T₅GAAGCGAAGTGC-3′ | HBV |
| 213 | 3′-CGCTGATTTGTC₅C₅G₅G₅G₅-5′-5′-gcgact | Angiotensinogen |
| 214 | 3′-CGCTGATTTGTCC₅G₅G₅G₅-5′-5′-gcgact | Angiotensinogen |
| 215 | 3′-CGCTGATTTGTC₅C₅G₅G₅G₅-5′-5′-gcgac | Angiotensinogen |
| 216 | 3′-CGCTGATTTGTCC₅G₅G₅G₅-5′-5′-gcgac | Angiotensinogen |
| 217 | 3′-CGCTGATTTGTC₅C₅G₅G₅G₅-5′-5′-g₅c₅g₅a₅c₅t₅ | Angiotensinogen |
| 218 | 3′-CGCTGATTTGTCC₅G₅G₅G₅-5′-5′-g₅c₅g₅a₅c₅t₅ | Angiotensinogen |
| 219 | gtcaaa-5′-5′-A₅C₅G₅G₅C₅A₅T₅T₅GGTGCACAGTTT-3′ | Factor XI |
| 220 | gtcaaa-5′-5′-A₅C₅G₅G₅C₅A₅T₅T₅G₅GTGCACAGTTT-3′ | Factor XI |
| 221 | tcaaa-5′-5′-A₅C₅G₅G₅C₅A₅T₅T₅GGTG₅CACAGTTT-3′ | Factor XI |
| 222 | tcaaa-5′-5′-A₅C₅G₅G₅C₅A₅T₅T₅G₅GTGCACAGTTT-3′ | Factor XI |
| 223 | cgtcga-5′-5′-C₅T₅T₅T₅A₅T₅T₅C₅CAAAGGGCAGCT-3′ | TMPRSS6 |
| 224 | cgtcga-5′-5′-C₅T₅T₅T₅A₅T₅T₅C₅C₅AAAGGGCAGCT-3′ | TMPRSS6 |
| 225 | gtcga-5′-5′-C₅T₅T₅T₅A₅T₅T₅C₅CAAAGGGCAGCT-3′ | TMPRSS6 |
| 226 | gtcga-5′-5′-CT₅T₅T₅A₅T₅T₅C₅C₅AAAGGGCAGCT-3′ | TMPRSS6 |
| 227 | c₅g₅t₅c₅g₅a₅-5′-5′-C₅T₅T₅T₅A₅T₅T₅C₅CAAAGGGCAGCT-3′ | TMPRSS6 |
| 228 | C₅g₅t₅c₅g₅a₅-5′-5′-C₅T₅T₅T₅A₅T₅T₅C₅C₅AAAGGGCAGCT-3′ | TMPRSS6 |
| 229 | agccaa-5′-5′-C₅A₅C₅A₅A₅A₅C₅A₅A₅GCTGGTCGGTT-3′ | Angiotensinogen |
| | | (AGT) |
| 230 | agccaa-5′-5′-C₅A₅C₅A₅A₅A₅C₅A₅AGCTGGTCGGTT-3′ | Angiotensinogen |
| | | (AGT) |
| 231 | gccaa-5′-5′-C₅A₅C₅A₅A₅A₅C₅A₅A₅GCTGGTCGGTT-3′ | Angiotensinogen |
| | | (AGT) |
| 232 | gccaa-5′-5′-C₅A₅C₅A₅A₅A₅C₅A₅AGCTGGTCGGTT-3′ | Angiotensinogen |
| | | (AGT) |
| 233 | gtggga-5′-5′-C₅C₅GT₅T₅T₅T₅CTTACCACCCT-3′ | MAPT |
| 234 | gtggga-5′-5′-C₅C₅G₅T₅T₅T₅TCTTACCACCCT-3′ | MAPT |
| 235 | tggga-5′-5′-C₅C₅G₅T₅T₅T₅T₅CTTACCACCCT-3′ | MAPT |
| 236 | tggga-5′-5′-C₅C₅G₅T₅T₅T₅TCTTACCACCCT-3′ | MAPT |
| 237 | accggt-5′-5′-A₅T₅G₅G₅T₅T₅T₅A₅TTCCATGGCCA-3′ | PTP1B (PTPN1) |
| 238 | ccggt-5′-5′-A₅T₅G₅G₅T₅T₅T₅A₅TTCCATGGCCA-3′ | PTP1B (PTPN1) |
| 239 | accggt-5′-5′-A₅T₅G₅G₅T₅T₅T₅ATTCCATGGCCA-3′ | PTP1B (PTPN1) |
| 240 | ccggt-5′-5′-A₅T₅G₅G₅T₅T₅T₅ATTCCATGGCCA-3′ | PTP1B (PTPN1) |
| 241 | 3′-GGCACAAGGGCA₅C₅A₅G₅A₅C₅U₅U₅U₅C₅-5′-5′-ccgtgt | HTT |
| 242 | 3′-GGCACAAGGGCAC₅A₅G₅A₅C₅U₅U₅U₅C₅-5′-5′-ccgtgt | HTT |
| 243 | 3′-GGCACAAGGGCA₅C₅A₅G₅A₅C₅U₅U₅U₅C₅-5′-5′-ccgtg | HTT |
| 244 | 3′-GGCACAAGGGCAC₅A₅G₅A₅C₅U₅U₅U₅C₅-5′-5′-ccgtg | HTT |
| 245 | 3′-GGCACAAGGGCA₅C₅A₅G₅A₅C₅U₅U₅U₅C₅-5′-5′-c₅c₅g₅t₅g₅t₅ | HTT |
| 246 | 3′-GGCACAAGGGCAC₅A₅G₅A₅C₅U₅U₅U₅C₅-5′-5′-c₅c₅g₅t₅g₅t₅ | HTT |
| 247 | tagcgt-5′-5′-G₅G₅A₅C₅A₅T₅T₅G₅C₅CAGTAATCGCA-3′ | ANGPTL3 |
| 248 | tagcgt-5′-5′-G₅G₅A₅C₅A₅T₅T₅G₅CCAGTAATCGCA-3′ | ANGPTL3 |
| 249 | agcgt-5′-5′-G₅G₅A₅C₅A₅T₅T₅G₅C₅CAGTAATCGCA-3′ | ANGPTL3 |
| 250 | agcgt-5′-5′-G₅G₅A₅C₅A₅T₅T₅G₅CCAGTAATCGCA-3′ | ANGPTL3 |
| 251 | ggttgt-5′-5′-C₅T₅T₅C₅A₅C₅A₅T₅T₅TCGATCCAACA-3′ | ATXN2 |
| 252 | ggttgt-5′-5′-C₅T₅T₅C₅A₅C₅A₅T₅TTCGATCCAACA-3′ | ATXN2 |
| 253 | gttgt-5′-5′-C₅T₅T₅C₅A₅C₅A₅T₅TTCGATCCAACA-3′ | ATXN2 |
| 254 | gttgt-5′-5′-C₅T₅T₅C₅A₅C₅A₅T₅TTCGATCCAACA-3′ | ATXN2 |
| 255 | ctaatag-5′-5′-A₅G₅T₅T₅G₅G₅A₅G₅C₅A₅A₅G₅A₅T₅TATC-3′ | SCNIA |
| 256 | taatag-5′-5′-A₅G₅T₅T₅G₅G₅A₅G₅C₅A₅A₅G₅A₅T₅TATC-3′ | SCNIA |
| 257 | aatag-5′-5′-A₅G₅T₅T₅G₅G₅A₅G₅C₅A₅A₅G₅A₅T₅TATC-3′ | SCNIA |
| 258 | aaggac-5′-5′-C₅A₅A₅T₅G₅C₅C₅A₅T₅C₅C₅T₅G₅G₅A₅G₅T₅T₅CCTG-3′ | DMD |
| 259 | aggac-5′-5′-C₅A₅A₅T₅G₅C₅C₅A₅T₅C₅C₅T₅G₅G₅A₅G₅T₅T₅CCTG-3′ | DMD |
| 260 | cacaag-5′-5′-G₅T₅T₅G₅C₅C₅T₅C₅C₅G₅G₅T₅T₅C₅T₅G₅A₅A₅G₅G₅T₅GTTC- | DMD |
| | 3′ | |
| 261 | acaag-5-5′-G₅T₅T₅G₅C₅C₅T₅C₅C₅G₅GT₅T₅C₅T₅G₅A₅A₅G₅GST₅GTTC-3′ | DMD |
| 262 | aagatc-5′-5′- | DMD |
| | CST₅C₅C₅A₅A₅C₅A₅T₅C₅A₅A₅G₅G₅A₅A₅G₅A₅T₅G₅G₅C₅A₅T₅T₅T₅CTAG- | |
| | 3′ | |
| 263 | agatc-5′-5′- | DMD |
| | C₅T₅C₅C₅A₅A₅C₅A₅T₅C₅A₅A₅G₅G₅A₅A₅G₅A₅T₅G₅G₅C₅A₅T₅T₅T₅CTAG- | |
| | 3′ | |
| 264 | T₂G₂T₂C₂G₂A₂-5′-5′-C₁A₁G₁G₁AIT₁A₁C₁ATTTCTACAGCT | SOD1 |
| 265 | T₂G₂T₂C₂G₂A₂-5′-5′-C₁A₁G₁GA₁T₁A₁CATTTCTACAGCT | SOD1 |
| 266 | T₂G₂T₂C₂G₂A₂-5′-5′-C₃A₃G₃G₃A₃T₃A₃C₃ATTTCTACAGCT | SODI |
| 267 | T₂G₂T₂C₂G₂A₂-5′-5′-C₃A₃G₃G₃A₃T₃A₃CATTTCTACAGCT | SOD1 |
| 268 | T₂G₂T₂C₂G₂A₂-5′-5′-C₁A₄G₁G₄A₁T₄A₁C₁ATTTCTACAGCT | SOD1 |
| 269 | T₂G₂T₂C₂G₂A₂-5′-5′-C₁A₄G₁G₄A₁T₄A₁CATTTCTACAGCT | SOD1 |
| 270 | T₂G₂T₂C₂G₂A₂-5′-5′-C₃A₄G₃G₄A₃T₄A₃C₃ATTTCTACAGCT | SOD1 |
| 271 | T₂G₂T₂C₂G₂A₂-5′-5′-C₃A₄G₃G₄A₃T₄A₃CATTTCTACAGCT | SOD1 |
| 272 | T₂G₂T₂C₂G₂A₂-5′-5′-C₁A₁G₁G₁A₁T₁A₁C₁ATTTCTACAGCT | SODI |
| 273 | T₂G₂T₂C₂G₂A₂-5′-5′-C₁A₁G₁GA₁T₁A₁CATTTCTACAGCT | SOD1 |
| 274 | T₂G₂T₂C₂G₂A₂-5′-5′-C₃A₃G₃G₃A₃T₃A₃C₃ATTTCTACAGCT | SOD1 |
| 275 | T₂G₂T₂C₂G₂A₂-5′-5′-C₃A₃G₃G₃A₃T₃A₃CATTTCTACAGCT | SOD1 |
| 276 | T₂G₂T₂C₂G₂A₂-5′-5′-C₁A₄G₁G₄A₁T₄A₁C₁ATTTCTACAGCT | SOD1 |
| 277 | T₂G₂T₂C₂G₂A₂-5′-5′-C₁A₄G₁G₄A₁T₄A₁CATTTCTACAGCT | SOD1 |
| 278 | T₂G₂T₂C₂G₂A₂-5′-5′-C₃A₄G₃G₄A₃T₄A₃C₃ATTTCTACAGCT | SODI |
| 279 | T₂G₂T₂C₂G₂A₂-5′-5′-C₃A₄G₃G₄A₃T₄A₃CATTTCTACAGCT | SOD1 |
| 337 | 3′-gtggtg-5′-5′-C₃T₃C₃A₃G₃T₃A₃A₃C₃ATTGACACCAC-3′ | HTT |
| 338 | 3′-gtggtg-5′-5′-C₃T₃C₃A₃G₃T₃A₃A₃C₃ATTGACACCAC-3′ | HTT |
| 339 | 3′-tggtg-5′-5′-C₃T₃C₃A₃G₃T₃A₃A₃C₃ATTGACACCAC-3′ | HTT |
| 340 | 3′-gtggtg-5′-5′-C₄T₃C₄A₃G₄T₃A₄A₃C₃ATTGACACCAC-3′ | HTT |
| 341 | 3′-tggtg-5′-5′-C₄T₃C₄A₃G₄T₃A₄A₃C₃ATTGACACCAC-3′ | HTT |
| 342 | 3′-gtggtg-5′-5′-C₄T₃C₄A₃G₄T₃A₄A₃C₄atTGACACCAC-3′ | HTT |
| 343 | 3′-gtggtg-5′-5′-C₄T₃C₄A₃G₄T₃A₄A₃C₃attGACACCAC-3′ | HTT |
| 344 | 3′-gtggtg-5′-5′-C₄T₃C₄A₃G₄T₃A₄A₃C₃AttgACACCAC-3′ | HTT |
| 345 | 3′-gtggtg-5′-5′-C₄T₃C₄A₃G₄T₃A₄A₃C₃atTGACACCAC-3′ | HTT |
| 346 | 3′-gtggtg-5′-5′-C₄T₃C₄A₃G₄T₃A₄A₃C₃AttGACACCAC-3′ | HTT |
| 347 | 3′-tggtg-5′-5′-C₄T₃C₄A₃G₄T₃A₄A₃C₄atTGACACCAC-3′ | HTT |
| 348 | 3′-tggtg-5′-5′-C₄T₃C₄A₃G₄T₃A₄A₃C₃attGACACCAC-3′ | HTT |
| 349 | 3′-tggtg-5′-5′-C₄T₃C₄A₃G₄T₃A₄A₃C₃AttgACACCAC-3′ | HTT |
| 350 | 3′-tggtg-5′-5′-C₄T₃C₄A₃G₄T₃A₄A₃C₃atTGACACCAC-3′ | HTT |
| 351 | 3′-tggtg-5′-5′-C₄T₃C₄A₃G₄T₃A₄A₃C₃AttGACACCAC-3′ | HTT |
| 352 | 3′-gtggga-5′-5′-C₃C₃G₃T₃T₃T₃T₃CTTACCACCCT-3′ | MAPT |
| 353 | 3′-tggga-5′-5′-C₃C₃G₃T₃T₃T₃T₃CTTACCACCCT-3′ | MAPT |
| 354 | 3′-gtggga-5′-5′-C₃C₃G₄T₄T₄T₄T₃CTTACCACCCT-3′ | MAPT |
| 355 | 3′-tggga-5′-5′-C₃C₃G₄T₄T₄T₄T₃CTTACCACCCT-3′ | MAPT |
| 356 | 3′-tgtcga-5′-5′-C₄A₄G₃G₃A₃T₃A₃C₃ATTTCTACAGCT-3′ | SOD1 |
| 357 | 3′-tgtcgA-5′-5′-C₄A₄G₃G₃A₃T₃A₃C₃ATTTCTACAGCT-3′ | SOD1 |
| 358 | 3′-tgtcga-5′-5′-C₄A₄G₄G₃A₃T₃A₃C₃ATTTCTACAGCT-3′ | SOD1 |
| 359 | 3′-tgtcga-5′-5′-C₄A₄G₄G₄A₃T₃A₃C₃ATTTCTACAGCT-3′ | SOD1 |
| 360 | 3′-TGtcga-5′-5′-C₄A₄G₄G₄A₃T₃A₃C₃ATTTCTACAGCT-3′ | SOD1 |
| 361 | 3′-tgtcgA-5′-5′-C₃A₄G₃G₄A₃TACATTTCTAC₄A₃G₄C₃T₃A₂C₂A₂G₂C₂T₂- | SOD1 |
| | 3′ | |
| 362 | 3′-tgtcga-5′-5′-C₄A₄G₃G₃A₃T₃A₃C₃ATTTCTACAGCTA₂C₂A₂G₂C₂T₂-3 | SOD1 |
| 363 | 3′-tgtcga-5′-5′-C₄A₄G₄G₃A₃T₃A₃C₃ATTTCTACAGCTA₂C₂A₂G₂C₂T₂-3′ | SOD1 |
| 364 | 3′-tcgtcc-5′-5′-C₃A₃G₃G₃A₃T₃A₃C₃A₃TTTCTACAGCTA₂G₂C₂A₂G₂G₂- | SOD1 |
| | 3′ | |
| 365 | 3′-tcgtcc-5′-5′-C₄A₄G₄G₃A₃T₃A₃C₃A₃TTTCTACAGCTA₂G₂C₂A₂G₂G₂- | SOD1 |
| | 3′ | |
| 366 | 3′-tcgtcc-5′-5′- | SOD1 |
| | C₃A₃G₃G₃A₃TACATTTCTAC₃A₃G₃C₃T₃T₂A₂G₂C₂A₂G₂G₂-3′ | |
| 367 | 3′-tcgtcc-5′-5′- | SOD1 |
| | C₃A₄G₃G₄A₃TACATTTCTAC₄A₃G₄C₃T₃T₂A₂G₂C₂A₂G₂G₂-3′ | |
| 368 | 3′-aaataa-5′-5′-G₃C₃T₃T₃C₃T₃T₃G₃T₃CCAGCTTTATT-3′ | APOC3 |
| 369 | 3′-Gaaataa-5′-5′-G₃C₃T₃T₃C₃T₃T₃G₃T₃CCAGCTTTATT-3′ | APOC3 |
| 370 | 3′-AAataa-5′-5′-G₃C₃T₃T₃C₃T₃T₃G₃T₃CCAGCTTTATT-3′ | APOC3 |
| 371 | 3′-GAAataa-5′-5′-G₃C₃T₃T₃C₃T₃T₃G₃T₃CCAGCTTTATT-3′ | APOC3 |
| 372 | 3′-aaataa-5′-5′-G₄C₄T₄T₃C₃T₃T₃G₃T₃CCAGCTTTATT-3′ | APOC3 |
| 373 | 3′-AAataa-5′-5′-G₄C₄T₄T₃C₃T₃T₃G₃T₃CCAGCTTTATT-3′ | APOC3 |
| 374 | 3′-aaataa-5′-5′-G₃C₃T₃T₃C₃T₃T₃G₃T₃CCAGCTTTATT-3′ | APOC3 |
| 375 | 3′-accctc-5′-5′-G₃C₃T₃T₃C₃T₃T₃G₃T₃CCAGCTTTATTG₂G₂G₂A₂G₂-3′ | APOC3 |
| 376 | 3′-accctc-5′-5′-G₄C₄T₄T₃C₃T₃T₃G₃T₃CCAGCTTTATTG₂G₂G₂A₂G₂-3′ | APOC3 |
| 377 | 3′-ACcctc-5′-5′-G₄C₄T₄T₃C₃T₃T₃G₃T₃CCAGCTTTATTG₂G₂G₂A₂G₂-3′ | APOC3 |
| 378 | 3′-ACCctc-5′-5′-G₄C₄T₄T₃C₃T₃T₃G₃T₃CCAGCTTTATTG₂G₂G₂A₂G₂- | APOC3 |
| | 3′ | |

GCAT-DNA phosphorothioate linkage; G₁/C₁/A₁/T₁/U₁₋₂′OME phosphorothioate linkage; A₂/T₂/C₂/G₂-DNA phosphodiester linkage; G₃/C₃/A₃/T₃/U₃₋₂′MOE phosphorothioate linkage; G₄/C₄/A₄/T₄/U₄-2′MOE phosphodiester linkage; Gs/Cs/As/Ts/Us-either 2′OME phosphorothioate linkage or 2′MOE phosphorothioate linkage; lowercase g/c/a/t—is phosphodiester DNA

### Pharmaceutical Composition

In certain embodiments, described herein are pharmaceutical compositions comprising one or more CSO compounds of the invention. In certain embodiments, the pharmaceutical composition comprises a pharmaceutically acceptable diluent or carrier. In certain embodiments, a pharmaceutical composition comprises a sterile saline solution and one or more CSO compounds. In certain embodiments, a pharmaceutical composition consists of a sterile saline solution and one or more CSO compounds. In certain embodiments, the sterile saline is pharmaceutical grade saline. In certain embodiments, a pharmaceutical composition comprises one or more CSO compounds and sterile water. In certain embodiments, a pharmaceutical composition consists of one CSO compound and sterile water. In certain embodiments, the sterile water is pharmaceutical grade water. In certain embodiments, a pharmaceutical composition comprises one or more CSO compounds and phosphate-buffered saline (PBS). In certain embodiments, a pharmaceutical composition consists of one or more CSO compounds and sterile PBS. In certain embodiments, the sterile PBS is pharmaceutical grade PBS.

In certain embodiments, pharmaceutical compositions comprise one or more CSO compounds and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, CSO compounds of the invention may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

### Conjugate Groups

In certain embodiments, the CSOs according to the invention optionally further comprise one or more conjugate groups. Conjugate groups consist of one or more conjugate moiety and a conjugate linker which links the one or more conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups are attached at the 3′-end of oligonucleotides. In certain embodiments, conjugate groups are attached near the 3′-end of oligonucleotides. In certain embodiments, conjugate groups are attached at the 5′-end of oligonucleotides. In certain embodiments, conjugate groups are attached near the 5′-end of oligonucleotides.

In any embodiment herein, the conjugate group comprises a GalNAc cluster comprising 1-3 GalNAc ligands.

In any embodiment herein, the conjugate linker consists of a single bond.

In any embodiment herein, the conjugate linker is cleavable.

In any embodiment herein, the conjugate linker comprises 1-3 linker-nucleosides.

In any embodiment herein, wherein the conjugate group is attached to the CSO at the 5′-end of the functional domain. In any embodiment herein, wherein the conjugate group is attached to the CSO at the 3′-end of the functional domain. In any embodiment herein, wherein the conjugate group is attached to the CSO at the 5′-end of the cyclizing domain. In any embodiment herein, wherein the conjugate group is attached to the CSO at the 3′-end of the cyclizing domain.

In certain embodiments, the CSOs are covalently attached to one or more conjugate groups. In certain embodiments, conjugate groups modify one or more properties of the CSO, including but not limited to pharmacodynamics, pharmacokinetics, stability, binding, absorption, tissue distribution, cellular distribution, cellular uptake, charge and clearance. In certain embodiments, conjugate groups impart a new property on the CSO, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycerol-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

Conjugate moieties also include antibodies. In embodiments the antibody is a transferrin receptor (TFR) antibody. The use of antibody conjugates can play a role in delivering CPNs of the invention and/or in cell targeting.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Conjugate moieties are attached to the CSO through conjugate linkers. In certain embodiments, the conjugate linker is a single chemical bond (i.e., the conjugate moiety is attached directly to the CSO through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on the CSO and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

As used herein, linker-nucleosides are not considered to be part of the CSO in general or part of the cyclizing domain or the functional domain in particular. Accordingly, the nucleotides of a linker-nucleosides are not counted toward the length of the CSO or the domains thereof and are not used in determining the percent complementarity of the oligonucleotide for the reference nucleic acid. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the CSO. For example, in certain circumstances CSOs comprising a particular conjugate moiety are better taken up by a particular cell type, but once the CSO has been taken up, it is desirable that the conjugate group be cleaved to release the unconjugated or parent CSO. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

### Use

Cyclic structured oligonucleotides could be useful as, or to deliver (e.g., to a target, or into a target environment (e.g., cytoplasm)), antisense oligonucleotides for mRNA, ncRNA, microRNA, lncRNA and splicing. Furthermore, cyclic structure could provide a novel approach to deliver antisense to disrupt structures of RNA to increase translation. For example, when the functional domain is an antisense oligonucleotide, CSOs according to the invention are also useful in therapeutic approaches in which inhibition of gene expression is desired. This can include, for example, inhibition of an endogenous gene (e.g., an oncogene) or an exogenous gene (e.g., a gene essential for growth and/or metabolism of a pathogen).

In embodiments, the invention provides a method for inhibiting gene expression comprising administering a cyclic structured oligonucleotide as described herein or a composition comprising the cyclic structured oligonucleotide.

In embodiments, the invention provides a method for inhibiting allele-specific gene expression comprising administering a cyclic structured oligonucleotide as described herein or a composition comprising the cyclic structured oligonucleotide.

The method according to the invention are useful for treating a subject having disease or disorder wherein inhibiting expression of a gene would be beneficial. In embodiments, the disease or disorder results from abnormal expression or product of a cellular gene.

In embodiments, the cyclic structured oligonucleotide as described herein or a composition comprising the cyclic structured oligonucleotide is administered locally.

In embodiments, the cyclic structured oligonucleotide as described herein or a composition comprising the cyclic structured oligonucleotide is administered systemically.

In embodiments, the invention provides a method of treating a disease or disorder in a subject wherein modulating RNA processing would be beneficial to treat the subject, the method comprising administering a cyclic structured oligonucleotide as described herein or a composition comprising the cyclic structured oligonucleotide.

In embodiments, the invention provides a method of inducing nonsense mediated decay of a target RNA comprising administering a cyclic structured oligonucleotide as described herein or a composition comprising the cyclic structured oligonucleotide.

The cyclic structured oligonucleotide of the invention may be administered alone or in combination with any other agent or therapy. Agents or therapies can be co-administered or administered concomitantly. Such agent or therapy may be useful for treating or preventing the disease or condition and does not diminish the gene expression modulation effect of the cyclic oligonucleotide according to the invention. Agent(s) useful for treating or preventing the disease or condition includes, but is not limited to, small molecules, peptides vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the cyclic oligonucleotide according to the invention can be administered in combination with other compounds (for example lipids or liposomes) to enhance the specificity or magnitude of the gene expression modulation of the cyclic oligonucleotide according to the invention.

The cyclic oligonucleotide of the invention may be administered by any suitable route, including, without limitation, parenteral, mucosal delivery, oral, sublingual, transdermal, topical, inhalation, intratumoral, intravenous, subcutaneous, intrathecal, intranasal, aerosol, intraocular, intratracheal, intrarectal, vaginal, by gene gun, dermal patch or in eye drop or mouthwash form. In any of the methods according to the invention, administration of the cyclic oligonucleotide according to the invention, alone or in combination with any other agent, can be directly to a tissue or organ such as, but not limited to, the bladder, liver, lung or kidney. In certain embodiments, administration of the cyclic oligonucleotide according to the invention, alone or in combination with any other agent, is by intramuscular administration. In certain embodiments, administration of antisense oligonucleotides according to the invention, alone or in combination with any other agent, is by mucosal administration. In certain embodiments, administration of antisense oligonucleotides according to the invention, alone or in combination with any other agent, is by oral administration. In certain embodiments, administration of antisense oligonucleotides according to the invention, alone or in combination with any other agent, is by intrarectal administration. In certain embodiments, administration of antisense oligonucleotides according to the invention, alone or in combination with any other agent, is by intrathecal administration. In certain embodiments, administration of antisense oligonucleotides according to the invention, alone or in combination with any other agent, is by intratumoral administration. In certain embodiments, administration of antisense oligonucleotides according to the invention, alone or in combination with any other agent, is by parenteral administration. In certain embodiments, administration of antisense oligonucleotides according to the invention, alone or in combination with any other agent, is by subcutaneous administration.

In embodiments, any of the cyclic structured oligonucleotides described herein can be conjugated with a moiety that provides for site specific delivery of the CSO. In embodiments, such conjugates include, but are not limited to, an antibody, a peptide, a lipid, or a small molecule.

In embodiments, any of the cyclic structured oligonucleotides described herein can be encapsulated with a moiety that provides for site specific delivery of the CSO. In embodiments, the CSO can be encapsulated in, for example, a lipid, lipid nanoparticles (LNP), or peptide macrocyclic structures.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Administration of the antisense oligonucleotides according to the invention can be carried out using known procedures using an effective amount and for periods of time effective to reduce symptoms or surrogate markers of the disease. For example, an effective amount of an antisense oligonucleotide according to the invention for treating a disease and/or disorder could be that amount necessary to alleviate or reduce the symptoms, or delay or ameliorate a tumor, cancer, or bacterial, viral or fungal infection. In the context of administering a composition that modulates gene expression, an effective amount of an antisense oligonucleotide according to the invention is an amount sufficient to achieve the desired modulation as compared to the gene expression in the absence of the antisense oligonucleotide according to the invention. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular antisense oligonucleotide without necessitating undue experimentation.

### Synthesis

CSOs described herein can be prepared by any suitable art recognized method including, but not limited to, H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles), which can be carried out manually or by an automated synthesizer. The oligonucleotides of the invention may also be modified in a number of ways without compromising their ability to hybridize to their target (see e.g., Agrawal and Gait, Advances in Nucleic Acid Therapeutics, (2019) https://doi.org/10.1039/9781788015714).

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2 ‘-substituted nucleoside” means a nucleoside comprising a 2’-substituted sugar moiety. As used herein, “2 ‘-substituted” in reference to a sugar moiety means a sugar moiety comprising at least one 2’-substituent group other than H or OH.

As used herein, “5-methyl cytosine” means a cytosine modified with a methyl group attached to the 5-position. A 5-methyl cytosine is a modified nucleobase.

As used herein, the singular forms “a”, “an”, and “the” include both singular and plural referents unless the context clearly dictates otherwise.

As used herein, “administering” means providing a pharmaceutical agent to an animal.

As used herein, “animal” means a human or non-human animal.

As used herein, “individual in need thereof” refers to a human or non-human animal selected for treatment or therapy that is in need of such treatment or therapy.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is an increase in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is ataxia, neuropathy, and aggregate formation. In certain embodiments, amelioration of these symptoms results in improved motor function, reduced neuropathy, or reduction in number of aggregates.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “cleavable moiety” means a bond or group of atoms that is cleaved under physiological conditions, for example, inside a cell, an animal, or a human.

As used herein, the term “complementary” refers to a pair of nucleobases (or simply a “base”) that hydrogen bond to each other in preference to other heterocyclic bases under selected (e.g., physiological) conditions (or some degree of complementarity thereof as context may require in the instance of assessing “complementary-ness” of oligonucleotides). When the nucleobases are modified or unmodified, natural or synthetic purines and pyrimidines, the term “complementary” means complementary in the Watson Crick sense. In embodiments, at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases refers to nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “linker-nucleoside” means a nucleoside that links, either directly or indirectly, the CSO of the invention to a conjugate moiety. Linker-nucleosides are located within the conjugate linker and are not considered part of the CSO compound even if they are contiguous with the CSO.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid amenable to oligomeric compounds for reducing the amount or activity of the target nucleic acid as demonstrated in the examples herein below.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, the phrase “inhibiting the expression or activity” refers to a reduction or blockade of the expression or activity relative to the expression of activity in an untreated or control sample and does not necessarily indicate a total elimination of expression or activity.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethyl. “2′-MOE” means a 2′-OCH₂CH₂OCH₃ group in place of the 2′ OH group of a ribosyl sugar moiety.

As used herein, “motif” means the pattern of unmodified and/or modified sugar moieties, nucleobases, and/or internucleoside linkages, in an oligonucleotide.

As used herein, “mRNA” means an RNA transcript that encodes a protein and includes pre-mRNA and mature mRNA unless otherwise specified.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is an example of a modified nucleobase. A universal base is a modified nucleobase that can pair with any one of the five unmodified nucleobases. Modified bases, also referred to as heterocyclic base moieties, include other nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine ([5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. In certain embodiments, the modified nucleobase is a 5-methylcytosine.

Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an antisense oligonucleotide may have one or more nucleotides with a sugar modification at one position and also one or more nucleotides with a sugar modification at a different position.

Sugar modifications contemplated in an oligonucleotide include, but are not limited to, a sugar substituent group selected from: OH; F; O—, S—, or N-alkyl; O—, S—, or N-alkenyl; O—, S or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)ₓCH₃, O(CH₂)ₓONH₂, and O(CH₂)ₓON((CH₂)ₓCH₃) 2, where x and y are independently from 1 to 10.

In some embodiments, the modified sugar comprises a substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF3, OCF₃, SOCH₃, SO₂CH₃, ONO, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an antisense oligonucleotide, or a group for improving the pharmacodynamic properties of an antisense oligonucleotide, and other substituents having similar properties. In one embodiment, the modification includes 2′-methoxyethoxy (2′-O—CH₂CH₂OCH₃, which is also known as 2′-O-(2-methoxyethyl) or 2′-MOE) (Martin et al., 1995), that is, an alkoxyalkoxy group. Another modification includes 2′-dimethylaminooxyethoxy, that is, a O(CH₂)₂ON(CH₃) 2 group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃) 2.

Additional sugar substituent groups include allyl (—CH₂—CH═CH₂), —O-allyl CH₂—CH═CH₂), methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics, for example, cyclobutyl moieties, in place of the pentofuranosyl sugar. Examples of U.S. patents that disclose the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, which are herein incorporated by reference in its entirety.

Representative sugar substituent groups include groups described in U.S. Patent Application Publication 2005/0261218, which is hereby incorporated by reference. In particular embodiments, the sugar modification is a 2′-O-Me modification, a 2′ F modification, a 2′ H modification, a 2′ amino modification, a 4′ thioribose modification or a phosphorothioate modification on the carboxy group linked to the carbon at position 6′, or combinations thereof.

In certain embodiments, a 2′-substituted non-bicyclic modified nucleoside comprises a sugar moiety comprising a non-bridging 2 ‘-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4’ and the 2′ furanose ring atoms. Examples of such 4′ to 2′ bridging sugar substituents include but are not limited to: 4′-CH₂-2′, 4′-(CH₂) 2-2′, 4′-(CH₂) 3-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al, U.S. Pat. No. 7,569,686, Swayze et al., U.S. Pat. No. 7,741,457, and Swayze et al, U.S. Pat. No. 8,022,193), 4′-C(CH₃) (CH₃)—O-2′ and analogs thereof (see, e.g., Seth et al., U.S. Pat. No. 8,278,283), 4′-CH₂—N(OCH₃)-2′ and analogs thereof (see, e.g., Prakash et al, U.S. Pat. No. 8,278,425), 4′-CH₂—O—N(CH₃)-2′ (see, e.g., Allerson et al., U.S. Pat. No. 7,696,345 and Allerson et al, U.S. Pat. No. 8,124,745), 4′-CH₂—C(H) (CH₃)-2′ (see, e.g., Zhou, et al, J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4-C(RₐR_{b})—O—N(R)-2′, 4′-CH₂—O—N(R)-2′, and 4′-CH₂—N(R)—O-2′, wherein each R, Rₐ and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ) (R_{b})]ₙ—, —[C(Rₐ) (R_{b})]ₙ—O—, —C(Rₐ)—C(R_{b})—, —C(Rₐ)—N—, —C(═NR^{a})—, —C(═O)—, —C(═S)—, —O—, —Si(Rₐ)₂—, —S(—O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25 (22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc, 2017, 129, 8362-8379; Wengel et al., U.S. Pat. No. 7,053,207; Imanishi et al., U.S. Pat. No. 6,268,490; Imanishi et al., U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the a-L configuration or in the β-D configuration.

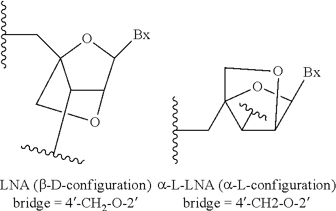

α-L-methyleneoxy (4′-CH₂-0-2′) or a-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5 ‘-substituted and 4’-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

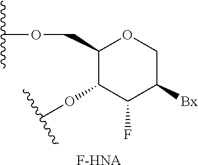

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

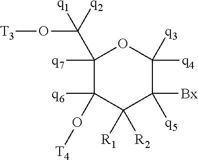

wherein, independently, for each of said modified THP nucleoside:
Bx is a nucleobase moiety;
T3 and T4 are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X) J₁, OC(═X) NJ₁J₂, NJ₃C(═X) NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R² is F. In certain embodiments, R₁ is F and R₂ is H, in certain embodiments, R₁ is methoxy and R₂ is H, and in certain embodiments, R¹ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

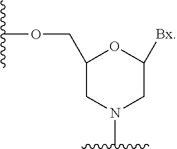

In certain embodiments, morpholinos may be modified, for example by adding or altering various substituent groups from the above morpholino structure. Such sugar surrogates are referred to herein as “modified morpholinos.” In embodiments, the morpholino or modified morpholino can further comprise a modified backbone, such as thiomorpholino or phosphorodiamidate morpholino (PMO), which are morpholino nucleoside(s) joined by thiophosphoramidate or phosphorodiamidate internucleotide linkages.

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

The nucleoside residues of the oligonucleotides of the functional or cyclizing domains can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”).

Representative non-phosphorus containing internucleoside linking groups include but are not limited to methylenemethylimino (—CH₂—N(CH₃)—O—CH₂—), thiodiester, thionocarbamate (—O—C(═O) (NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art.

Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. In some embodiments, the synthetic antisense oligonucleotides of the invention may comprise combinations of internucleotide linkages. In some embodiments, the synthetic antisense oligonucleotides of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration.

In certain embodiments, the phosphorothioate linkages may be mixed Rp and Sp enantiomers, or they may be made stereoregular or substantially stereoregular in either Rp or Sp form. In embodiments where the linkages are mixed Rp and Sp enantiomers, the Rp and Sp forms may be at defined places within the oligonucleotide or randomly placed throughout the oligonucleotide.

As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, (Devereux, J., et al., Nucleic Acids Research, 12 (1), 387 (1984)), BLASTP, BLASTN, and FASTA (Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within an animal or cells thereof. Typically, conversion of a prodrug within the animal is facilitated by the action of an enzyme (e.g., endogenous or viral enzyme) or chemicals present in cells or tissues and/or by physiologic conditions.

As used herein, “OMe” means methoxy. “2′-OMe” means a 2′-OCH₃ group in place of the 2′ OH group of a ribosyl sugar moiety.

The recitation of numerical ranges by endpoints includes all numbers and fractions subsumed within the respective ranges, as well as the recited endpoints.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the(S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

As used herein, “treat”, “treatment”, or “treating” refers to administering a compound described herein to effect an alteration or improvement of a disease, disorder, or condition.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating a disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “patient” generally refers to a mammal, such as a human. The term “mammal” is expressly intended to include warm blooded, vertebrate animals, including, without limitation, humans, non-human primates, rats, mice, cats, dogs, horses, cattle, cows, pigs, sheep and rabbits. As used herein, “individual in need thereof refers to a human or non-human animal selected for treatment or therapy that is in need of such treatment or therapy. As used herein, “inhibiting the expression or activity” refers to a reduction or blockade of the expression or activity of a RNA or protein and does not necessarily indicate a total elimination of expression or activity.

### EXAMPLES

### Synthesis of CSO Comprising an Antisense Oligonucleotide Functional Domain

Cyclic structured oligonucleotides according to the invention can be synthesized by procedures that are well known in the art, such as phosphoramidate or H-phosphonate chemistry which can be carried out manually or by an automated synthesizer. For example, the oligonucleotides of the invention may be synthesized by a linear synthesis approach.

Compounds employed herein have been synthesized using phosphoramidite chemistry. These protocols are described in detail, for example in pubs.rsc.org/en/content/chapter/bk9781788012096-00453/978-1-78801-209-6, which is incorporated herein by reference.

### Inhibition of Target RNA by CSO Comprising an Antisense Oligonucleotide Functional Domain

Cyclic structured oligonucleotides were designed targeting a sequence of nucleotides within APOB, TTR, APOC3, PTPIB, PCSK9, STAT3, ANGPTL3, KLKB1 DGAT2, HTT, MAPT, SOD1, or HPRT1 and were tested for their effects on mRNA expression in vitro.

Hep3B cells (human hepatic origin) or U-251 MG cells were cultured following ATCC recommended conditions and media (Eagle's Minimum Essential Medium with 10% FBS). Cells were plated in 96 well plates at 20K/well density and reverse transfected with 0.3 ul/well RNAiMax and cyclic structured antisense oligonucleotides (CSOs) at six different concentrations (25, 6.25, 1.56, 0.39, 0.10, and 0.024 nM). Control wells were transfected with 0.3 ul/well RNAiMax and media alone or 25 nM non-template control antisense oligonucleotides and incubated for 48 hours post-transfection. Gene expression was assayed using the Invitrogen™ TaqMan™ Fast Advanced Cells-to-CT™ Kit following the manufacturer's protocol (A35378, Invitrogen™). In brief, cells were harvested using the Cells to CT lysis reagent and immediately following total RNA was reverse transcribed into cDNA using the RT reagents. Quantitative polymerase chain reaction (qPCR) was performed via multiplexed reactions using qPCR Fast Advanced Master Mix and pre-designed primers and FAM-labelled probes (4351370, Invitrogen™) for the corresponding gene of interest (GOI) and normalized using pre-designed primers and VIC-labeled probes (#4448486, Invitrogen™) for the reference gene Hypoxanthine-guanine phosphoribosyltransferase (HPRT1). Data was plotted and analyzed using the ‘Absolute IC50’ nonlinear regression dose-response model of GraphPad Prism.

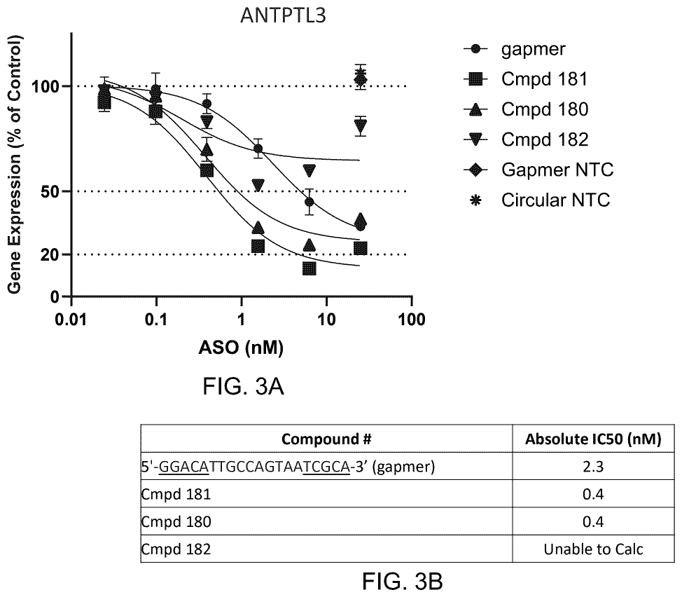

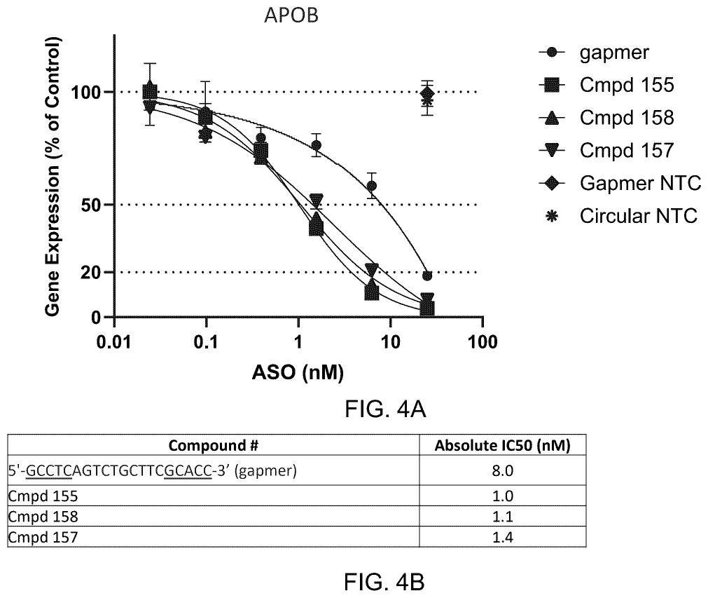

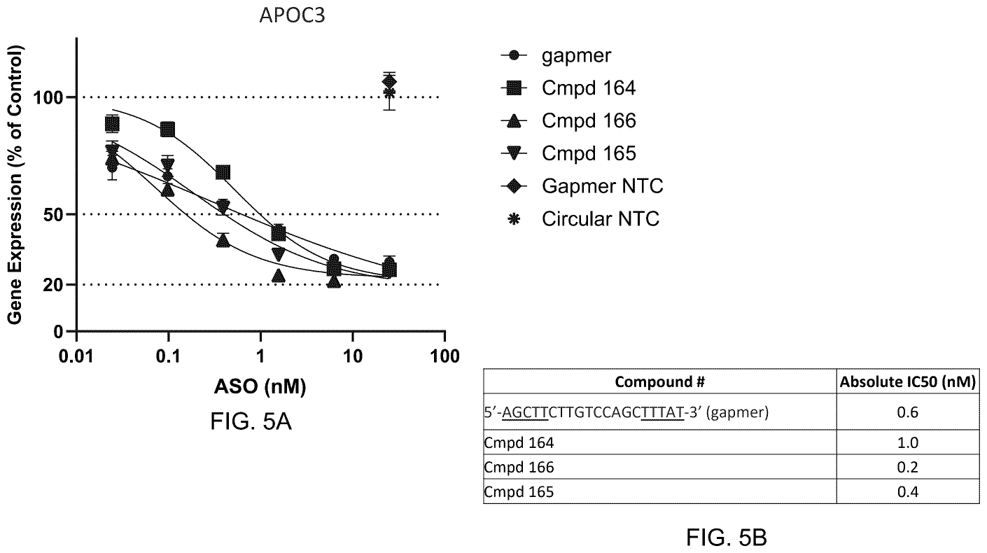

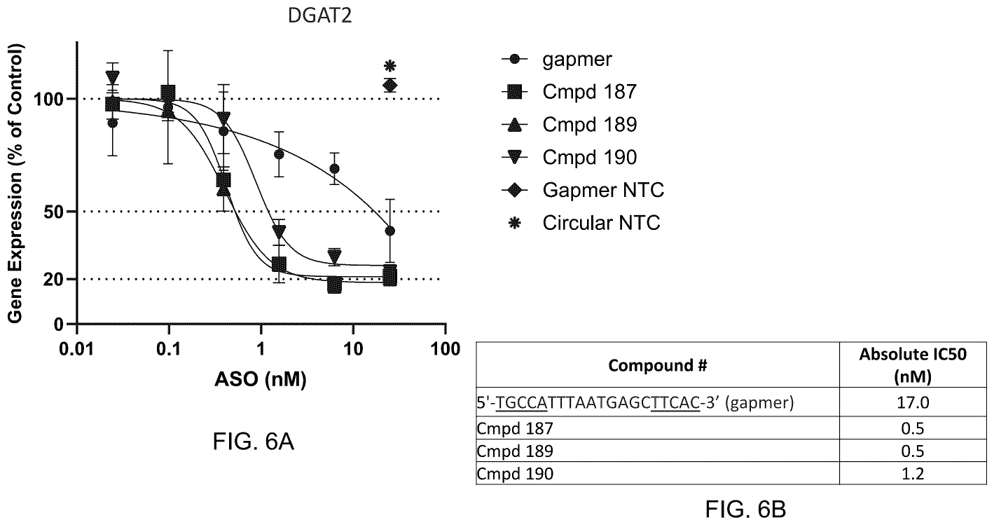

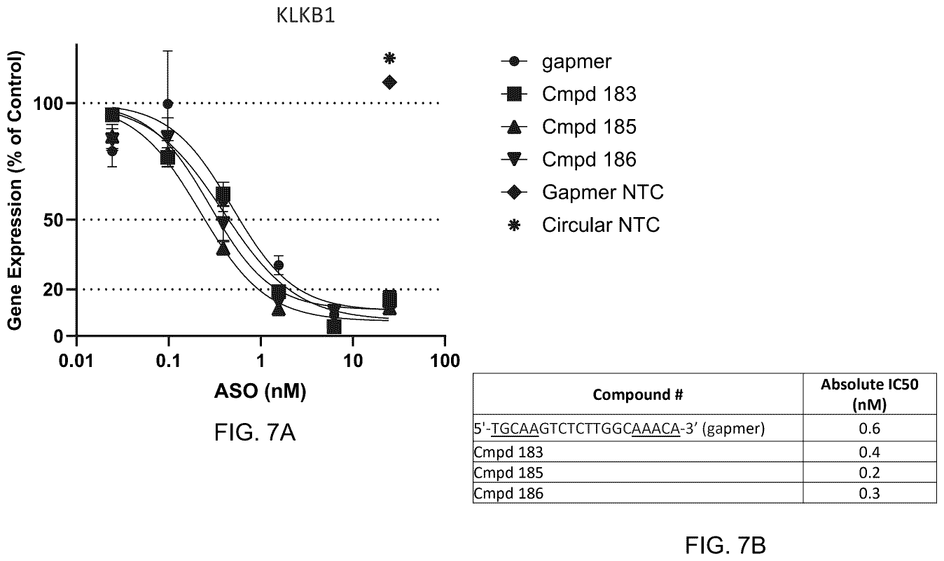

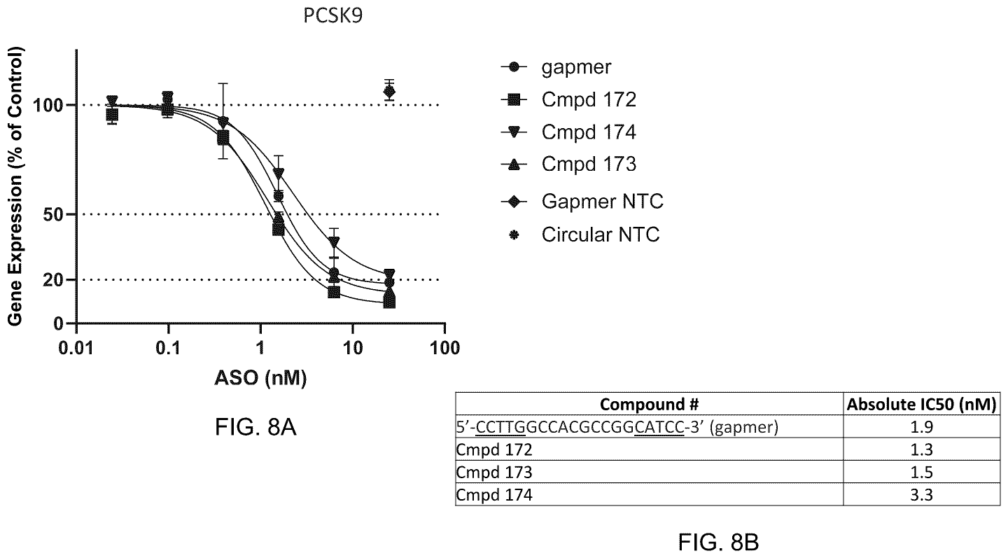

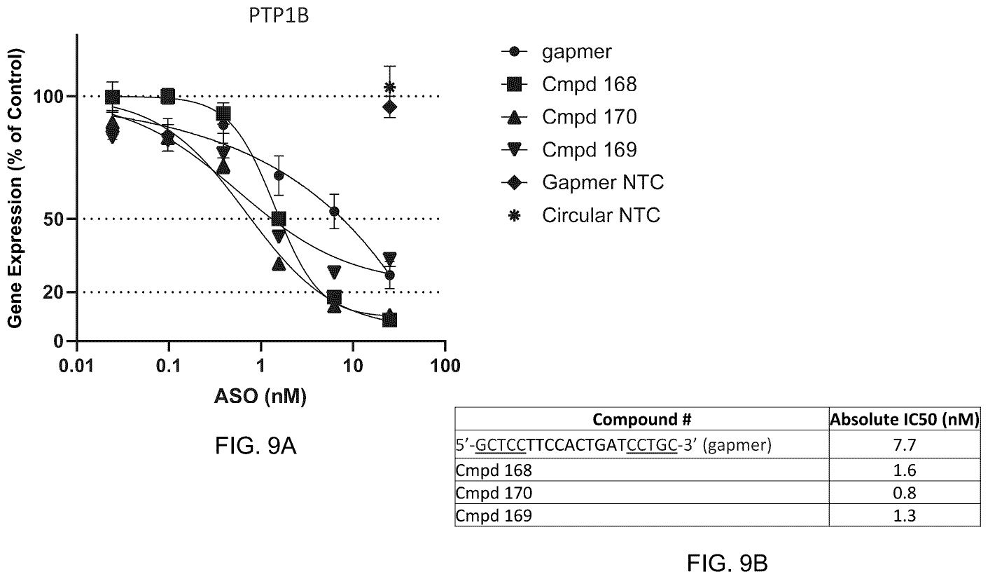

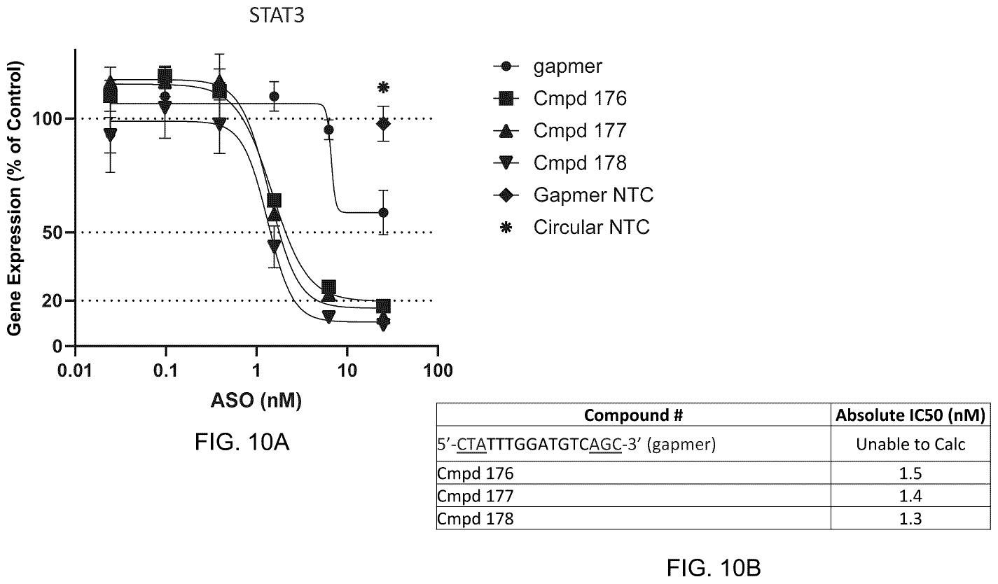

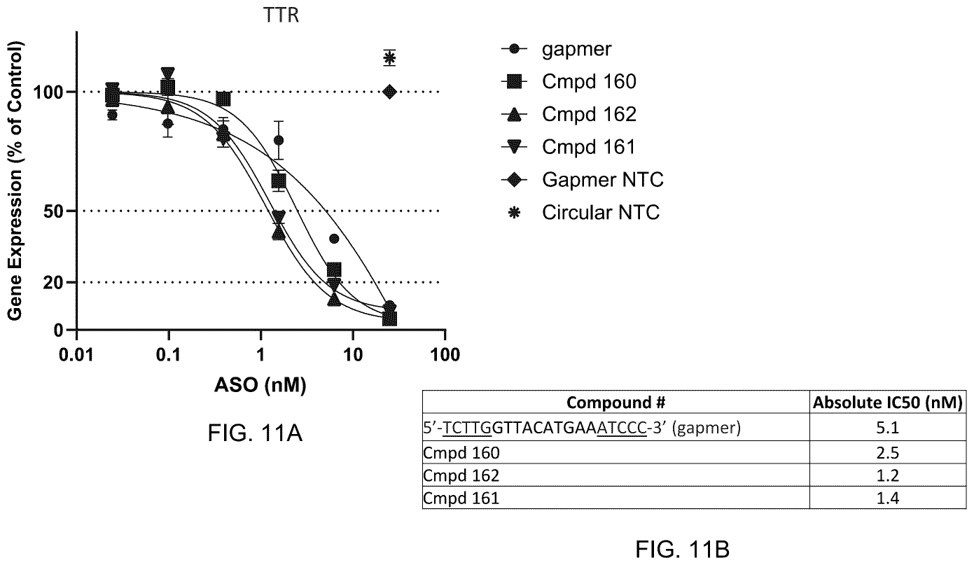

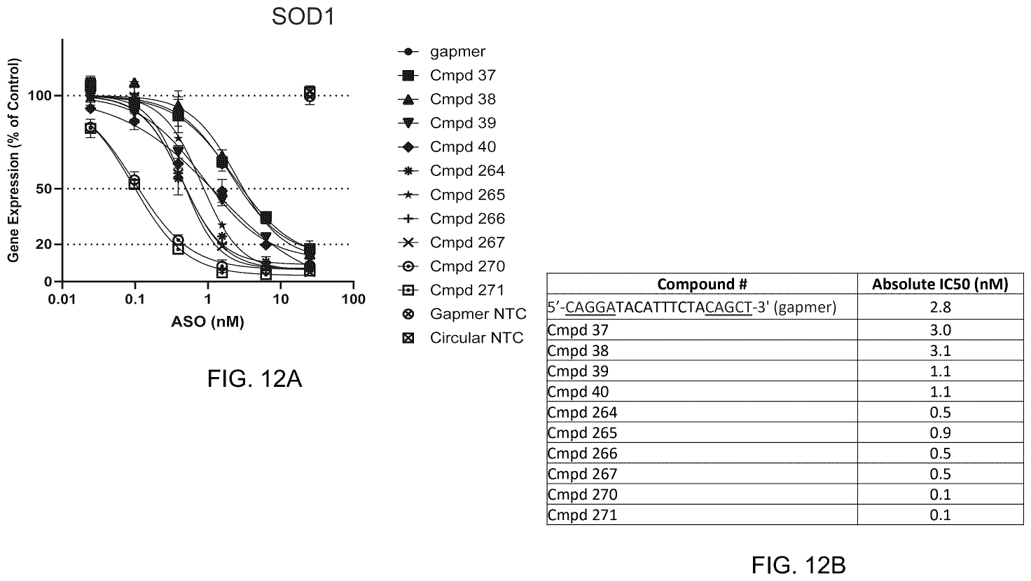

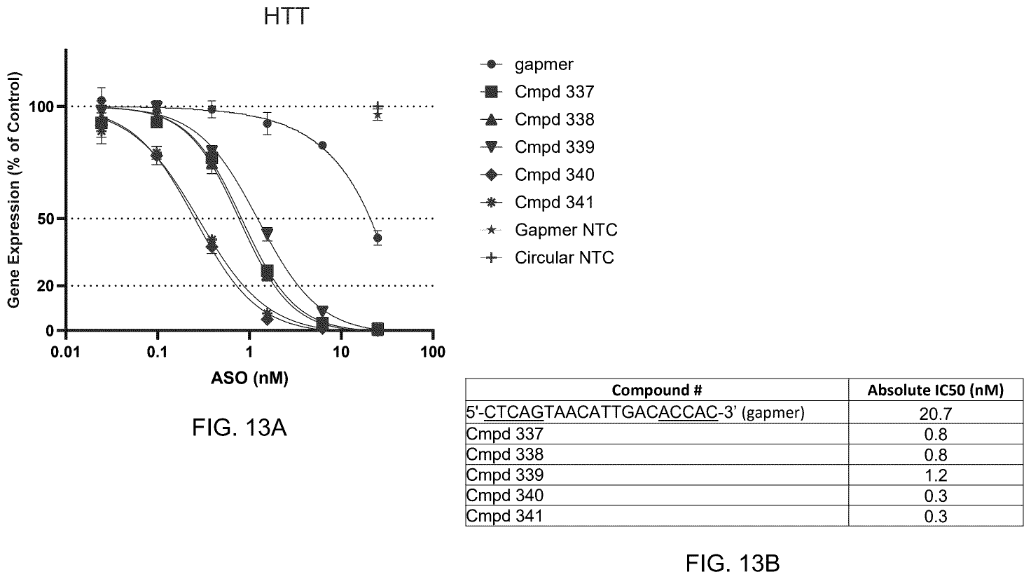

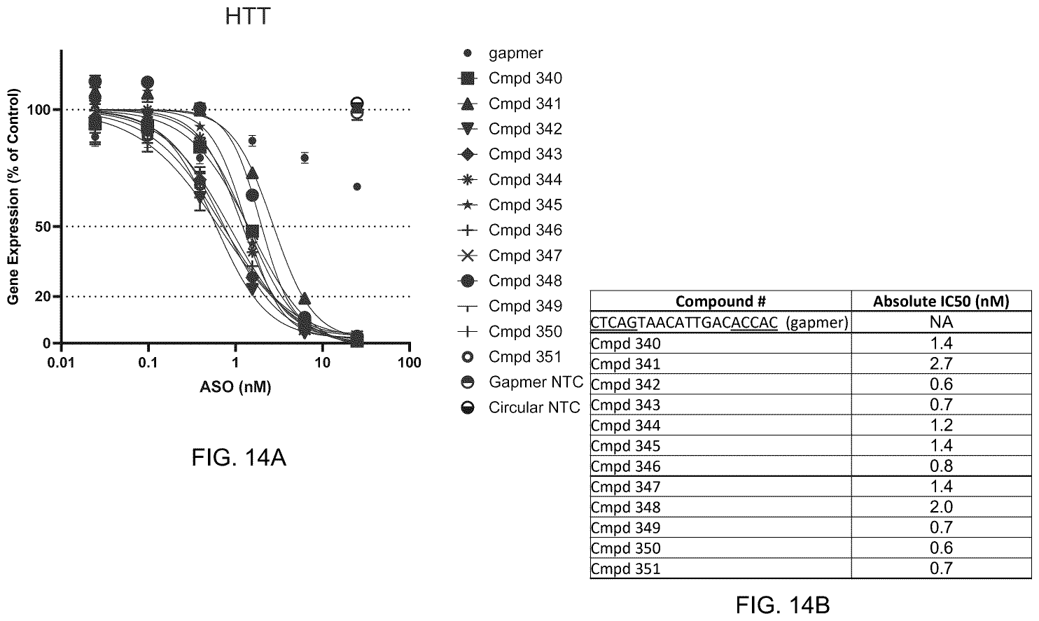

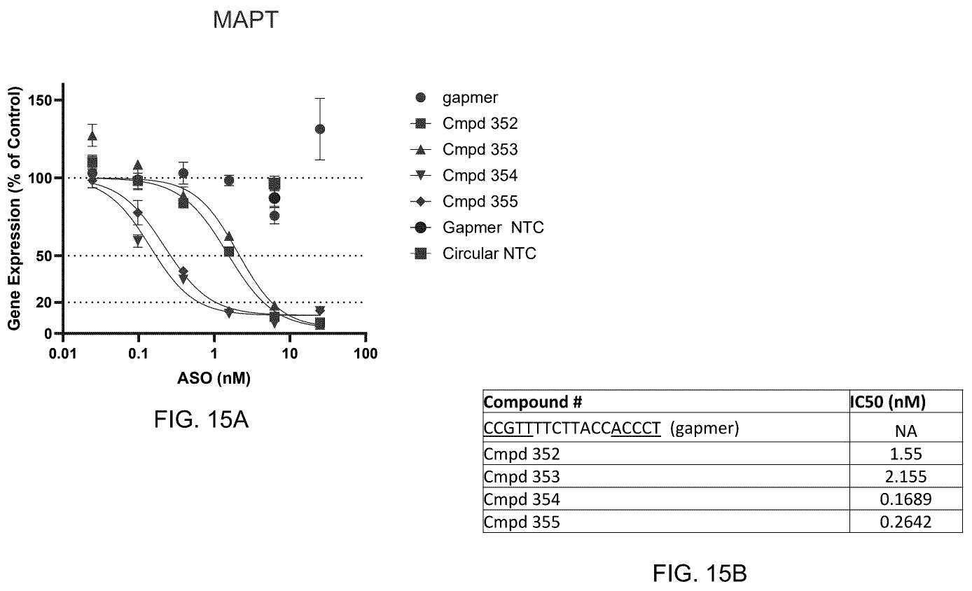

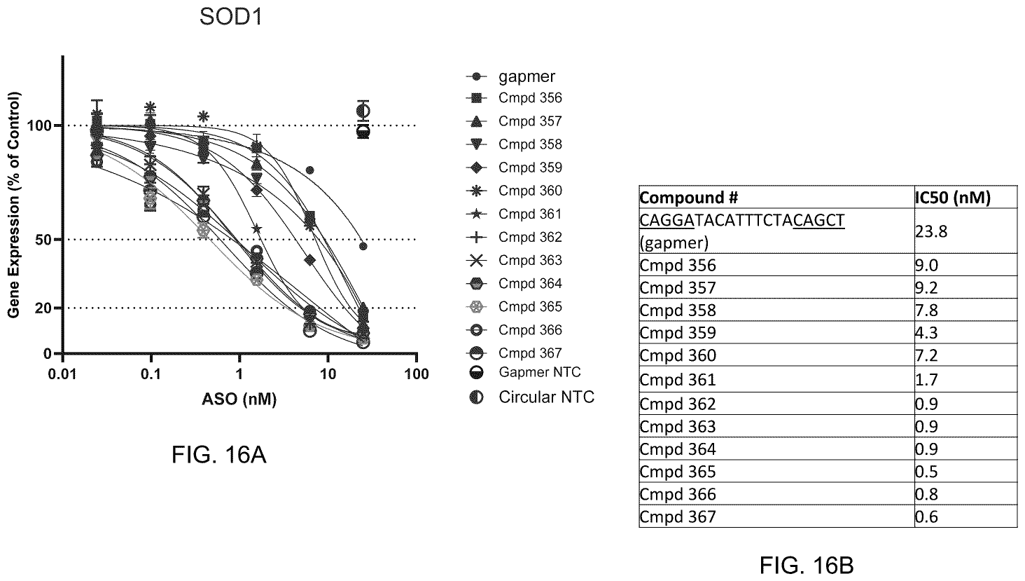

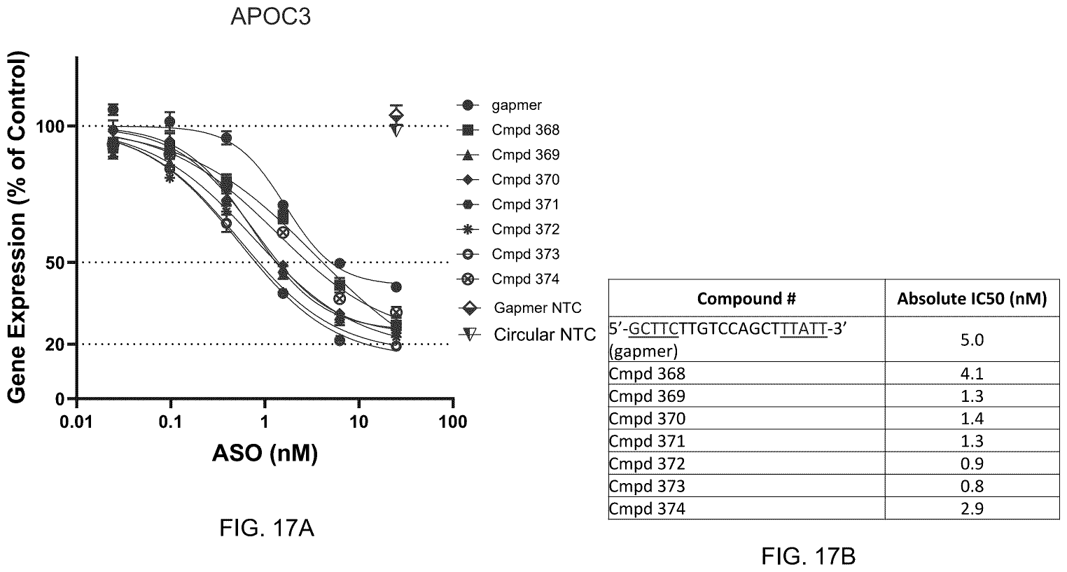

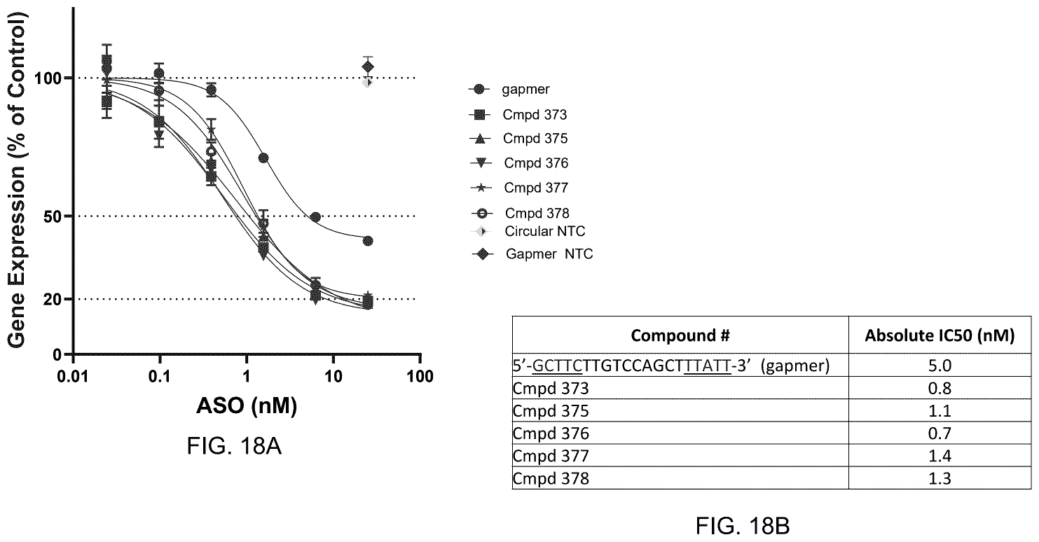

FIGS. 3-18 show a comparison of control, clinical candidate antisense oligonucleotides sequences in gapmer format with three different circularized formats. The same sequence was used to design a linear gapmer formatted ASO and three different permutations of a circularized formatted CSO. Hep3B cells or U-251 MG cells were transfected with indicated oligonucleotides and concentrations (25, 6.25, 1.56, 0.39, 0.10, and 0.024 nM) and harvested 48 hours later for gene expression analysis. Data are plotted as percent of control cells (cells reverse-transfected with vehicle alone). Table of IC50 and IC80 values calculated using the absolute IC50 nonlinear regression dose-response model of Graphpad Prism are also provided. Gapmer non-target control:

(SEQ ID NO: 339)
5′-CCAAATCTTATAATAACTAC-3′

Circular non-target control:

(SEQ ID NO: 340)
3′-ttgatg-5′-5′-CCAAATCTTATAATAACTAC-3′

Underline represents 2′-MOE ribonucleotide.

While this invention has been particularly shown and described with references to preferred embodiments thereof, it will be understood by those skilled in the art that various changes in form and details may be made therein without departing from the scope of the invention encompassed by the appended claims.

1. A cyclic structured oligonucleotide (CSO) comprising a functional domain and a cyclizing domain, wherein the functional domain and the cyclizing domain are linked at their 5′ ends; wherein the cyclizing domain comprises an oligonucleotide between 4 and 12 nucleotides in length and is complementary to a sequence of nucleotides within the functional domain and of polarity opposite to the sequence of nucleotides within the functional domain to which it is complementary; wherein the cyclizing domain hybridizes with the functional domain, thereby forming a cyclic structure; wherein the functional domain comprises an antisense oligonucleotide, wherein the antisense oligonucleotide is at least 80% identical to an antisense oligonucleotide selected from Table 1 and comprising at least 12 contiguous nucleobases complementary to an equal length portion of a target RNA sequence.

2. The CSO according to claim 1, wherein the antisense oligonucleotide comprises a 3′ domain and a 5′ domain, which is contiguous with the 3′ domain,
wherein the 3′ domain begins at the terminal nucleotide at the 3′ end and is 10 to 12 nucleotides in length and wherein each nucleotide is unmodified and wherein each internucleotide linkage between adjacent nucleotides is a phosphodiester or phosphorothioate internucleotide linkage or combinations thereof; and
wherein the 5′ domain begins at the first nucleotide following the 3′ domain and continues to the terminal nucleotide at the 5′ end, wherein at least 3 nucleotides of 5′ domain comprise a modified sugar and/or backbone; wherein the modified deoxyribonucleotides and/or modified ribonucleotides of the 5′ domain need not be consecutive; and wherein the modified deoxyribonucleotides and/or modified ribonucleotides of the 5′ domain prevent RNase H cleavage in the 5′ domain.

3. The CSO according to claim 2, wherein at least half of the nucleotides of the 5′ domain comprise a modified sugar and/or a modified backbone.

4. The CSO according to claim 2, wherein all of the nucleotides of the 5′ domain comprise a modified sugar and/or backbone.

5. The CSO according to claim 1, wherein the cyclizing domain is 4 to 8 nucleotides in length.

6. The CSO according to claim 1, wherein the nucleotides of the cyclizing domain comprises unmodified deoxyribonucleotides, unmodified ribonucleotides, modified deoxyribonucleotides, modified ribonucleotides, or combinations thereof.

7. The CSO according to claim 6, wherein the nucleotides of the cyclizing comprise unmodified ribonucleotides.

8. The CSO according to claim 6, wherein the nucleotides of the cyclizing comprise unmodified deoxyribonucleotides.

9. The CSO according to claim 6, wherein the nucleotides of the cyclizing comprise modified ribonucleotides.

10. The CSO according to claim 9, wherein the modified ribonucleotides comprise 2′-substituted ribonucleotides.

11. The CSO according to claim 10, wherein the 2′-substituted ribonucleotides are 2′-OMe ribonucleotides or 2′-MOE ribonucleotides.

12. The CSO according to claim 1, wherein the internucleotidic linkages of the oligonucleotides of the functional domain and/or the cyclizing domain comprise phosphorothioate internucleotide linkages, phosphodiester internucleotide linkages, or combinations thereof.

13. The CSO according to claim 12, wherein the oligonucleotide of the functional domain comprises at least one phosphorothioate internucleotide linkage.

14. The CSO according to claim 13, wherein at least half of the internucleotide linkages are phosphorothioate linkages.

15. The CSO according to claim 13, wherein all the internucleotide linkages are phosphorothioate linkages.

16. The CSO according to claim 12, wherein all the internucleotide linkages are phosphodiester linkages.

17. The CSO according to claim 12, wherein the oligonucleotide of the cyclizing domain comprises at least one phosphorothioate internucleotide linkage.

18. The CSO according to claim 17, wherein at least half of the internucleotide linkages are phosphorothioate linkages.

19. The CSO according to claim 17, wherein all the internucleotide linkages are phosphorothioate linkages.

20. The CSO according to claim 12, wherein all the internucleotide linkages are phosphodiester linkages.

21. The CSO according to claim 1, wherein the functional domain comprises an antisense oligonucleotide comprising at least 80% sequence identity to an antisense oligonucleotide selected from Table 2.

22. The CSO according to claim 1, wherein the cyclizing domain is selected from Table 3.

23. The CSO according to claim 1, wherein the functional domain and the cyclizing domain are linked at their 5′ ends through a direct bond.

24. The CSO according to claim 1, wherein the CSO comprises an antisense oligonucleotide comprising at least 80% identical to an antisense oligonucleotide selected from Table 4.

25. A pharmaceutical composition comprising the antisense oligonucleotide according to claim 1 and a pharmaceutically acceptable carrier.

26. A method for inhibiting gene expression comprising administering a CSO according to claim 1, or a composition according to claim 25.

27. The method according to claim 26, wherein in the method is useful for treating a subject having disease or disorder wherein inhibiting expression of a gene would be beneficial.


SEQUENCE LISTING (340 sequences)

SEQ ID NO: 1 - DNA, 20 bp, synthetic construct
  source 1..20: mol_type = other DNA
  modified_base 1..8: mod_base = OTHER; note = 2' O-methyl ribonucleotide
gcctcagtctgcttcgcacc

SEQ ID NO: 2 - DNA, 20 bp, synthetic construct
  source 1..20: mol_type = other DNA
  modified_base 1..9: mod_base = OTHER; note = 2' O-methyl ribonucleotide
gcctcagtctgcttcgcacc

SEQ ID NO: 3 - DNA, 20 bp, synthetic construct
  source 1..20: mol_type = other DNA
  modified_base 1..8: mod_base = OTHER; note = 2' methoxyethyl ribonucleotide
gcctcagtctgcttcgcacc

SEQ ID NO: 4 - DNA, 20 bp, synthetic construct
  source 1..20: mol_type = other DNA
  modified_base 1..9: mod_base = OTHER; note = 2' methoxyethyl ribonucleotide
gcctcagtctgcttcgcacc

SEQ ID NO: 5 - DNA, 20 bp, synthetic construct
  source 1..20: mol_type = other DNA
  modified_base 1..8: mod_base = OTHER; note = 2' O-methyl ribonucleotide
tcttggttacatgaaatccc

SEQ ID NO: 6 - DNA, 20 bp, synthetic construct
  source 1..20: mol_type = other DNA
  modified_base 1..9: mod_base = OTHER; note = 2' O-methyl ribonucleotide
tcttggttacatgaaatccc

SEQ ID NO: 7 - DNA, 20 bp, synthetic construct
  source 1..20: mol_type = other DNA
  modified_base 1..8: mod_base = OTHER; note = 2' methoxyethyl ribonucleotide
tcttggttacatgaaatccc

SEQ ID NO: 8 - DNA, 20 bp, synthetic construct
  source 1..20: mol_type = other DNA
  modified_base 1..9: mod_base = OTHER; note = 2' methoxyethyl ribonucleotide
tcttggttacatgaaatccc

SEQ ID NO: 9 - DNA, 20 bp, synthetic construct
  source 1..20: mol_type = other DNA
  modified_base 1..8: mod_base = OTHER; note = 2' O-methyl ribonucleotide
agcttcttgtccagctttat

SEQ ID NO: 10 - DNA, 20 bp, synthetic construct
  source 1..20: mol_type = other DNA
  modified_base 1..9: mod_base = OTHER; note = 2' O-methyl ribonucleotide
agcttcttgtccagctttat

SEQ ID NO: 11 - DNA, 20 bp, synthetic construct
  source 1..20: mol_type = other DNA
  modified_base 1..8: mod_base = OTHER; note = 2' methoxyethyl ribonucleotide
agcttcttgtccagctttat

SEQ ID NO: 12 - DNA, 20 bp, synthetic construct
  source 1..20: mol_type = other DNA
  modified_base 1..9: mod_base = OTHER; note = 2' methoxyethyl ribonucleotide
agcttcttgtccagctttat

SEQ ID NO: 13 - DNA, 20 bp, synthetic construct
  source 1..20: mol_type = other DNA
  modified_base 1..8: mod_base = OTHER; note = 2' O-methyl ribonucleotide
gctccttccactgatcctgc

SEQ ID NO: 14 - DNA, 20 bp, synthetic construct
  source 1..20: mol_type = other DNA
  modified_base 1..9: mod_base = OTHER; note = 2' O-methyl ribonucleotide
gctccttccactgatcctgc

SEQ ID NO: 15 - DNA, 20 bp, synthetic construct
  source 1..20: mol_type = other DNA
  modified_base 1..8: mod_base = OTHER; note = 2' methoxyethyl ribonucleotide
gctccttccactgatcctgc

SEQ ID NO: 16 - DNA, 20 bp, synthetic construct
  source 1..20: mol_type = other DNA
  modified_base 1..9: mod_base = OTHER; note = 2' methoxyethyl ribonucleotide
gctccttccactgatcctgc

SEQ ID NO: 17 - DNA, 20 bp, synthetic construct
  source 1..20: mol_type = other DNA
  modified_base 1..8: mod_base = OTHER; note = 2' O-methyl ribonucleotide
ccttggccacgccggcatcc

SEQ ID NO: 18 - DNA, 20 bp, synthetic construct
  source 1..20: mol_type = other DNA
  modified_base 1..9: mod_base = OTHER; note = 2' O-methyl ribonucleotide
ccttggccacgccggcatcc

SEQ ID NO: 19 - DNA, 20 bp, synthetic construct
  source 1..20: mol_type = other DNA
  modified_base 1..8: mod_base = OTHER; note = 2' methoxyethyl ribonucleotide
ccttggccacgccggcatcc

SEQ ID NO: 20 - DNA, 20 bp, synthetic construct
  source 1..20: mol_type = other DNA
  modified_base 1..9: mod_base = OTHER; note = 2' methoxyethyl ribonucleotide
ccttggccacgccggcatcc

SEQ ID NO: 21 - DNA, 16 bp, synthetic construct
  source 1..16: mol_type = other DNA
  modified_base 1..4: mod_base = OTHER; note = 2' O-methyl ribonucleotide
ctatttggatgtcagc

SEQ ID NO: 22 - DNA, 16 bp, synthetic construct
  source 1..16: mol_type = other DNA
  modified_base 1..5: mod_base = OTHER; note = 2' O-methyl ribonucleotide
ctatttggatgtcagc

SEQ ID NO: 23 - DNA, 16 bp, synthetic construct
  source 1..16: mol_type = other DNA
  modified_base 1..4: mod_base = OTHER; note = 2' methoxyethyl ribonucleotide
ctatttggatgtcagc

SEQ ID NO: 24 - DNA, 16 bp, synthetic construct
  source 1..16: mol_type = other DNA
  modified_base 1..5: mod_base = OTHER; note = 2' methoxyethyl ribonucleotide
ctatttggatgtcagc

SEQ ID NO: 25 - DNA, 20 bp, synthetic construct
  source 1..20: mol_type = other DNA
  modified_base 1..8: mod_base = OTHER; note = 2' O-methyl ribonucleotide
ggacattgccagtaatcgca

SEQ ID NO: 26 - DNA, 20 bp, synthetic construct
  source 1..20: mol_type = other DNA
  modified_base 1..9: mod_base = OTHER; note = 2' O-methyl ribonucleotide
ggacattgccagtaatcgca

SEQ ID NO: 27 - DNA, 20 bp, synthetic construct
  source 1..20: mol_type = other DNA
  modified_base 1..8: mod_base = OTHER; note = 2' methoxyethyl ribonucleotide
ggacattgccagtaatcgca

SEQ ID NO: 28 - DNA, 20 bp, synthetic construct
  source 1..20: mol_type = other DNA
  modified_base 1..9: mod_base = OTHER; note = 2' methoxyethyl ribonucleotide
ggacattgccagtaatcgca

SEQ ID NO: 29 - DNA, 20 bp, synthetic construct
  source 1..20: mol_type = other DNA
  modified_base 1..8: mod_base = OTHER; note = 2' O-methyl ribonucleotide
tgcaagtctcttggcaaaca

SEQ ID NO: 30 - DNA, 20 bp, synthetic construct
  source 1..20: mol_type = other DNA
  modified_base 1..9: mod_base = OTHER; note = 2' O-methyl ribonucleotide
tgcaagtctcttggcaaaca

SEQ ID NO: 31 - DNA, 20 bp, synthetic construct
  source 1..20: mol_type = other DNA
  modified_base 1..8: mod_base = OTHER; note = 2' methoxyethyl ribonucleotide
tgcaagtctcttggcaaaca

SEQ ID NO: 32 - DNA, 20 bp, synthetic construct
  source 1..20: mol_type = other DNA
  modified_base 1..9: mod_base = OTHER; note = 2' methoxyethyl ribonucleotide
tgcaagtctcttggcaaaca

SEQ ID NO: 33 - DNA, 20 bp, synthetic construct
  source 1..20: mol_type = other DNA
  modified_base 1..8: mod_base = OTHER; note = 2' O-methyl ribonucleotide
tgccatttaatgagcttcac

SEQ ID NO: 34 - DNA, 20 bp, synthetic construct
  source 1..20: mol_type = other DNA
  modified_base 1..9: mod_base = OTHER; note = 2' O-methyl ribonucleotide
tgccatttaatgagcttcac

SEQ ID NO: 35 - DNA, 20 bp, synthetic construct
  source 1..20: mol_type = other DNA
  modified_base 1..8: mod_base = OTHER; note = 2' methoxyethyl ribonucleotide
tgccatttaatgagcttcac

SEQ ID NO: 36 - DNA, 20 bp, synthetic construct
  source 1..20: mol_type = other DNA
  modified_base 1..9: mod_base = OTHER; note = 2' methoxyethyl ribonucleotide
tgccatttaatgagcttcac

SEQ ID NO: 37 - DNA, 20 bp, synthetic construct
  source 1..20: mol_type = other DNA
  modified_base 1..8: mod_base = OTHER; note = 2' O-methyl ribonucleotide
caggatacatttctacagct

SEQ ID NO: 38 - DNA, 20 bp, synthetic construct
  source 1..20: mol_type = other DNA
  modified_base 1..7: mod_base = OTHER; note = 2' O-methyl ribonucleotide
caggatacatttctacagct

SEQ ID NO: 39 - DNA, 20 bp, synthetic construct
  source 1..20: mol_type = other DNA
  modified_base 1..8: mod_base = OTHER; note = 2' methoxyethyl ribonucleotide
caggatacatttctacagct

SEQ ID NO: 40 - DNA, 20 bp, synthetic construct
  source 1..20: mol_type = other DNA
  modified_base 1..7: mod_base = OTHER; note = 2' methoxyethyl ribonucleotide
caggatacatttctacagct

SEQ ID NO: 41 - DNA, 20 bp, synthetic construct
  source 1..20: mol_type = other DNA
  modified_base 1..8: mod_base = OTHER; note = 2' methoxyethyl ribonucleotide
caggatacatttctacagct

SEQ ID NO: 42 - DNA, 20 bp, synthetic construct
  source 1..20: mol_type = other DNA
  modified_base 1..7: mod_base = OTHER; note = 2' methoxyethyl ribonucleotide
caggatacatttctacagct

SEQ ID NO: 43 - DNA, 20 bp, synthetic construct
  source 1..20: mol_type = other DNA
  modified_base 1..8: mod_base = OTHER; note = 2' O-methyl ribonucleotide
caggatacatttctacagct

SEQ ID NO: 44 - DNA, 20 bp, synthetic construct
  source 1..20: mol_type = other DNA
  modified_base 1..7: mod_base = OTHER; note = 2' O-methyl ribonucleotide
caggatacatttctacagct

SEQ ID NO: 45 - DNA, 20 bp, synthetic construct
  source 1..20: mol_type = other DNA
  modified_base 1..8: mod_base = OTHER; note = 2' methoxyethyl ribonucleotide
caggatacatttctacagct

SEQ ID NO: 46 - DNA, 20 bp, synthetic construct
  source 1..20: mol_type = other DNA
  modified_base 1..7: mod_base = OTHER; note = 2' methoxyethyl ribonucleotide
caggatacatttctacagct

SEQ ID NO: 47 - DNA, 20 bp, synthetic construct
  source 1..20: mol_type = other DNA
caggatacatttctacagct

SEQ ID NO: 48 - DNA, 20 bp, synthetic construct
  source 1..20: mol_type = other DNA
caggatacatttctacagct

SEQ ID NO: 49 - DNA, 20 bp, synthetic construct
  source 1..20: mol_type = other DNA
  modified_base 1..8: mod_base = OTHER; note = 2' O-methyl ribonucleotide
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 50 - DNA, 20 bp, synthetic construct
  source 1..20: mol_type = other DNA
  modified_base 4: mod_base = OTHER; note = uracil
  modified_base 16: mod_base = OTHER; note = uracil
  modified_base 1..8: mod_base = OTHER; note = 2' methoxyethyl ribonucleotide
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 51 - DNA, 20 bp, synthetic construct
  source 1..20: mol_type = other DNA
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
  modified_base 1..9: mod_base = OTHER; note = 2' methoxyethyl ribonucleotide
ccatactttccactgtaacg

SEQ ID NO: 52 - DNA, 20 bp, synthetic construct
  source 1..20: mol_type = other DNA
  modified_base 1..9: mod_base = OTHER; note = 2' O-methyl ribonucleotide
tgctccgttggtgcttgttc

SEQ ID NO: 53 - DNA, 20 bp, synthetic construct
  source 1..20: mol_type = other DNA
  modified_base 1..8: mod_base = OTHER; note = 2' O-methyl ribonucleotide
tgctccgttggtgcttgttc

SEQ ID NO: 54 - DNA, 20 bp, synthetic construct
  source 1..20: mol_type = other DNA
  modified_base 1..9: mod_base = OTHER; note = 2' methoxyethyl ribonucleotide
tgctccgttggtgcttgttc

SEQ ID NO: 55 - DNA, 20 bp, synthetic construct
  source 1..20: mol_type = other DNA
  modified_base 1..8: mod_base = OTHER; note = 2' methoxyethyl ribonucleotide
tgctccgttggtgcttgttc

SEQ ID NO: 56 - DNA, 20 bp, synthetic construct
  source 1..20: mol_type = other DNA
  modified_base 1..9: mod_base = OTHER; note = 2' O-methyl ribonucleotide
  modified_base 2: mod_base = OTHER; note = uracil
  misc_feature 1: note = DNA
  misc_feature 2: note = RNA
  misc_feature 3..20: note = DNA
ctcagtaacattgacaccac

SEQ ID NO: 57 - DNA, 20 bp, synthetic construct
  source 1..20: mol_type = other DNA
  modified_base 1..10: mod_base = OTHER; note = 2' O-methyl ribonucleotide
  modified_base 2: mod_base = OTHER; note = uracil
  misc_feature 1: note = DNA
  misc_feature 2: note = RNA
  misc_feature 3..20: note = DNA
ctcagtaacattgacaccac

SEQ ID NO: 58 - DNA, 20 bp, synthetic construct
  source 1..20: mol_type = other DNA
  modified_base 2: mod_base = OTHER; note = uracil
  misc_feature 1: note = DNA
  misc_feature 2: note = RNA
  misc_feature 3..20: note = DNA
  modified_base 1..10: mod_base = OTHER; note = 2' methoxyethyl ribonucleotide
ctcagtaacattgacaccac

SEQ ID NO: 59 - DNA, 20 bp, synthetic construct
  source 1..20: mol_type = other DNA
  modified_base 2: mod_base = OTHER; note = uracil
  misc_feature 1: note = DNA
  misc_feature 2: note = RNA
  misc_feature 3..20: note = DNA
  modified_base 1..9: mod_base = OTHER; note = 2' methoxyethyl ribonucleotide
ctcagtaacattgacaccac

SEQ ID NO: 60 - DNA, 20 bp, synthetic construct
  source 1..20: mol_type = other DNA
  modified_base 1..9: mod_base = OTHER; note = 2' O-methyl ribonucleotide
gcagaggtgaagcgaagtgc

SEQ ID NO: 61 - DNA, 20 bp, synthetic construct
  source 1..20: mol_type = other DNA
  modified_base 1..8: mod_base = OTHER; note = 2' O-methyl ribonucleotide
gcagaggtgaagcgaagtgc

SEQ ID NO: 62 - DNA, 20 bp, synthetic construct
  source 1..20: mol_type = other DNA
  modified_base 1..9: mod_base = OTHER; note = 2' methoxyethyl ribonucleotide
gcagaggtgaagcgaagtgc

SEQ ID NO: 63 - DNA, 20 bp, synthetic construct
  source 1..20: mol_type = other DNA
  modified_base 1..8: mod_base = OTHER; note = 2' methoxyethyl ribonucleotide
gcagaggtgaagcgaagtgc

SEQ ID NO: 64 - DNA, 16 bp, synthetic construct
  source 1..16: mol_type = other DNA
  modified_base 1..5: mod_base = OTHER; note = 2' O-methyl ribonucleotide
gggcctgtttagtcgc

SEQ ID NO: 65 - DNA, 16 bp, synthetic construct
  source 1..16: mol_type = other DNA
  modified_base 1..4: mod_base = OTHER; note = 2' O-methyl ribonucleotide
gggcctgtttagtcgc

SEQ ID NO: 66 - DNA, 16 bp, synthetic construct
  source 1..16: mol_type = other DNA
  modified_base 1..5: mod_base = OTHER; note = 2' methoxyethyl ribonucleotide
gggcctgtttagtcgc

SEQ ID NO: 67 - DNA, 16 bp, synthetic construct
  source 1..16: mol_type = other DNA
  modified_base 1..4: mod_base = OTHER; note = 2' methoxyethyl ribonucleotide
gggcctgtttagtcgc

SEQ ID NO: 68 - DNA, 20 bp, synthetic construct
  source 1..20: mol_type = other DNA
  modified_base 1..9: mod_base = OTHER; note = 2' O-methyl ribonucleotide
cacaaacaagctggtcggtt

SEQ ID NO: 69 - DNA, 20 bp, synthetic construct
  source 1..20: mol_type = other DNA
  modified_base 1..8: mod_base = OTHER; note = 2' O-methyl ribonucleotide
cacaaacaagctggtcggtt

SEQ ID NO: 70 - DNA, 20 bp, synthetic construct
  source 1..20: mol_type = other DNA
  modified_base 1..9: mod_base = OTHER; note = 2' methoxyethyl ribonucleotide
cacaaacaagctggtcggtt

SEQ ID NO: 71 - DNA, 20 bp, synthetic construct
  source 1..20: mol_type = other DNA
  modified_base 1..8: mod_base = OTHER; note = 2' methoxyethyl ribonucleotide
cacaaacaagctggtcggtt

SEQ ID NO: 72 - DNA, 20 bp, synthetic construct
  source 1..20: mol_type = other DNA
  modified_base 1..8: mod_base = OTHER; note = 2' O-methyl ribonucleotide
acggcattggtgcacagttt

SEQ ID NO: 73 - DNA, 20 bp, synthetic construct
  source 1..20: mol_type = other DNA
  modified_base 1..9: mod_base = OTHER; note = 2' O-methyl ribonucleotide
acggcattggtgcacagttt

SEQ ID NO: 74 - DNA, 20 bp, synthetic construct
  source 1..20: mol_type = other DNA
  modified_base 1..8: mod_base = OTHER; note = 2' methoxyethyl ribonucleotide
  modified_base 12: mod_base = OTHER; note = 2' methoxyethyl ribonucleotide
acggcattggtgcacagttt

SEQ ID NO: 75 - DNA, 20 bp, synthetic construct
  source 1..20: mol_type = other DNA
  modified_base 1..9: mod_base = OTHER; note = 2' methoxyethyl ribonucleotide
acggcattggtgcacagttt

SEQ ID NO: 76 - DNA, 18 bp, synthetic construct
  source 1..18: mol_type = other DNA
  modified_base 1..14: mod_base = OTHER; note = 2' O-methyl ribonucleotide
tcactttcataatgctgg

SEQ ID NO: 77 - DNA, 18 bp, synthetic construct
  source 1..18: mol_type = other DNA
  modified_base 1..14: mod_base = OTHER; note = 2' methoxyethyl ribonucleotide
tcactttcataatgctgg

SEQ ID NO: 78 - DNA, 20 bp, synthetic construct
  source 1..20: mol_type = other DNA
  modified_base 1..8: mod_base = OTHER; note = 2' O-methyl ribonucleotide
ctttattccaaagggcagct

SEQ ID NO: 79 - DNA, 20 bp, synthetic construct
  source 1..20: mol_type = other DNA
  modified_base 1..9: mod_base = OTHER; note = 2' O-methyl ribonucleotide
ctttattccaaagggcagct

SEQ ID NO: 80 - DNA, 20 bp, synthetic construct
  source 1..20: mol_type = other DNA
  modified_base 1..8: mod_base = OTHER; note = 2' methoxyethyl ribonucleotide
ctttattccaaagggcagct

SEQ ID NO: 81 - DNA, 20 bp, synthetic construct
  source 1..20: mol_type = other DNA
  modified_base 1..9: mod_base = OTHER; note = 2' methoxyethyl ribonucleotide
ctttattccaaagggcagct

SEQ ID NO: 82 - DNA, 18 bp, synthetic construct
  source 1..18: mol_type = other DNA
  modified_base 1..7: mod_base = OTHER; note = 2' O-methyl ribonucleotide
ccgttttcttaccaccct

SEQ ID NO: 83 - DNA, 18 bp, synthetic construct
  source 1..18: mol_type = other DNA
  modified_base 1..6: mod_base = OTHER; note = 2' O-methyl ribonucleotide
ccgttttcttaccaccct

SEQ ID NO: 84 - DNA, 18 bp, synthetic construct
  source 1..18: mol_type = other DNA
  modified_base 1..7: mod_base = OTHER; note = 2' methoxyethyl ribonucleotide
ccgttttcttaccaccct

SEQ ID NO: 85 - DNA, 18 bp, synthetic construct
  source 1..18: mol_type = other DNA
  modified_base 1..6: mod_base = OTHER; note = 2' methoxyethyl ribonucleotide
ccgttttcttaccaccct

SEQ ID NO: 86 - DNA, 19 bp, synthetic construct
  source 1..19: mol_type = other DNA
  modified_base 1..8: mod_base = OTHER; note = 2' O-methyl ribonucleotide
atggtttattccatggcca

SEQ ID NO: 87 - DNA, 19 bp, synthetic construct
  source 1..19: mol_type = other DNA
  modified_base 1..7: mod_base = OTHER; note = 2' O-methyl ribonucleotide
atggtttattccatggcca

SEQ ID NO: 88 - DNA, 19 bp, synthetic construct
  source 1..19: mol_type = other DNA
  modified_base 1..8: mod_base = OTHER; note = 2' methoxyethyl ribonucleotide
atggtttattccatggcca

SEQ ID NO: 89 - DNA, 19 bp, synthetic construct
  source 1..19: mol_type = other DNA
  modified_base 1..7: mod_base = OTHER; note = 2' methoxyethyl ribonucleotide
atggtttattccatggcca

SEQ ID NO: 90 - DNA, 21 bp, synthetic construct
  source 1..21: mol_type = other DNA
  modified_base 1..10: mod_base = OTHER; note = 2' O-methyl ribonucleotide
  modified_base 2..4: mod_base = OTHER; note = uracil
  misc_feature 1: note = DNA
  misc_feature 2..4: note = RNA
  misc_feature 5..21: note = DNA
ctttcagacacgggaacacgg

SEQ ID NO: 91 - DNA, 21 bp, synthetic construct
  source 1..21: mol_type = other DNA
  modified_base 1..9: mod_base = OTHER; note = 2' O-methyl ribonucleotide
  modified_base 2..4: mod_base = OTHER; note = uracil
  misc_feature 1: note = DNA
  misc_feature 2..4: note = RNA
  misc_feature 5..21: note = DNA
ctttcagacacgggaacacgg

SEQ ID NO: 92 - DNA, 21 bp, synthetic construct
  source 1..21: mol_type = other DNA
  modified_base 1..10: mod_base = OTHER; note = 2' methoxyethyl ribonucleotide
  modified_base 2..4: mod_base = OTHER; note = uracil
  misc_feature 1: note = DNA
  misc_feature 2..4: note = RNA
  misc_feature 5..21: note = DNA
ctttcagacacgggaacacgg

SEQ ID NO: 93 - DNA, 21 bp, synthetic construct
  source 1..21: mol_type = other DNA
  modified_base 1..9: mod_base = OTHER; note = 2' methoxyethyl ribonucleotide
  modified_base 2..4: mod_base = OTHER; note = uracil
  misc_feature 1: note = DNA
  misc_feature 2..4: note = RNA
  misc_feature 5..21: note = DNA
ctttcagacacgggaacacgg

SEQ ID NO: 94 - DNA, 20 bp, synthetic construct
  source 1..20: mol_type = other DNA
  modified_base 1..9: mod_base = OTHER; note = 2' O-methyl ribonucleotide
cttcacatttcgatccaaca

SEQ ID NO: 95 - DNA, 20 bp, synthetic construct
  source 1..20: mol_type = other DNA
  modified_base 1..8: mod_base = OTHER; note = 2' O-methyl ribonucleotide
cttcacatttcgatccaaca

SEQ ID NO: 96 - DNA, 20 bp, synthetic construct
  source 1..20: mol_type = other DNA
  modified_base 1..9: mod_base = OTHER; note = 2' methoxyethyl ribonucleotide
cttcacatttcgatccaaca

SEQ ID NO: 97 - DNA, 20 bp, synthetic construct
  source 1..20: mol_type = other DNA
  modified_base 1..8: mod_base = OTHER; note = 2' methoxyethyl ribonucleotide
cttcacatttcgatccaaca

SEQ ID NO: 98 - DNA, 18 bp, synthetic construct
  source 1..18: mol_type = other DNA
  modified_base 1..14: mod_base = OTHER; note = 2' O-methyl ribonucleotide
agttggagcaagattatc

SEQ ID NO: 99 - DNA, 18 bp, synthetic construct
  source 1..18: mol_type = other DNA
  modified_base 1..14: mod_base = OTHER; note = 2' methoxyethyl ribonucleotide
agttggagcaagattatc

SEQ ID NO: 100 - DNA, 22 bp, synthetic construct
  source 1..22: mol_type = other DNA
  modified_base 1..18: mod_base = OTHER; note = 2' O-methyl ribonucleotide
caatgccatcctggagttcctg

SEQ ID NO: 101 - DNA, 22 bp, synthetic construct
  source 1..22: mol_type = other DNA
  modified_base 1..18: mod_base = OTHER; note = 2' methoxyethyl ribonucleotide
caatgccatcctggagttcctg

SEQ ID NO: 102 - DNA, 25 bp, synthetic construct
  source 1..25: mol_type = other DNA
  modified_base 1..21: mod_base = OTHER; note = 2' O-methyl ribonucleotide
gttgcctccggttctgaaggtgttc

SEQ ID NO: 103 - DNA, 25 bp, synthetic construct
  source 1..25: mol_type = other DNA
  modified_base 1..21: mod_base = OTHER; note = 2' methoxyethyl ribonucleotide
gttgcctccggttctgaaggtgttc

SEQ ID NO: 104 - DNA, 30 bp, synthetic construct
  source 1..30: mol_type = other DNA
  modified_base 1..26: mod_base = OTHER; note = 2' O-methyl ribonucleotide
ctccaacatcaaggaagatggcatttctag

SEQ ID NO: 105 - DNA, 30 bp, synthetic construct; identical to SEQ ID NO: 104
  source 1..30: mol_type = other DNA
  modified_base 1..26: mod_base = OTHER; note = 2' methoxyethyl ribonucleotide

SEQ ID NO: 106-153 - skipped (48 entries)

SEQ ID NO: 154 - DNA, 20 bp, synthetic construct
  source 1..20: mol_type = other DNA
  modified_base 1..9: mod_base = OTHER; note = 2' O-methyl ribonucleotide
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 155-196 - skipped (42 entries)

SEQ ID NO: 197 - DNA, 20 bp, synthetic construct
  source 1..20: mol_type = other DNA
  misc_feature 1..3: note = DNA
  misc_feature 4: note = RNA
  misc_feature 5..15: note = DNA
  misc_feature 16: note = RNA
  misc_feature 17..20: note = DNA
  modified_base 1..9: mod_base = OTHER; note = 2' O-methyl or 2' methoxyethyl ribonucleotide
  modified_base 4: mod_base = OTHER; note = uracil
  modified_base 16: mod_base = OTHER; note = uracil
ccatactttccactgtaacg

SEQ ID NO: 198 - DNA, 20 bp, synthetic construct
  source 1..20: mol_type = other DNA
  misc_feature 1..3: note = DNA
  misc_feature 4: note = RNA
  misc_feature 5..15: note = DNA
  misc_feature 16: note = RNA
  misc_feature 17..20: note = DNA
  modified_base 1..8: mod_base = OTHER; note = 2' O-methyl or 2' methoxyethyl ribonucleotide
  modified_base 4: mod_base = OTHER; note = uracil
  modified_base 16: mod_base = OTHER; note = uracil
ccatactttccactgtaacg

SEQ ID NO: 199 - DNA, 20 bp, synthetic construct
  source 1..20: mol_type = other DNA
  misc_feature 1..3: note = DNA
  misc_feature 4: note = RNA
  misc_feature 5..15: note = DNA
  misc_feature 16: note = RNA
  misc_feature 17..20: note = DNA
  modified_base 1..9: mod_base = OTHER; note = 2' O-methyl or 2' methoxyethyl ribonucleotide
  modified_base 4: mod_base = OTHER; note = uracil
  modified_base 16: mod_base = OTHER; note = uracil
ccatactttccactgtaacg

SEQ ID NO: 200 - DNA, 20 bp, synthetic construct
  source 1..20: mol_type = other DNA
  misc_feature 1..3: note = DNA
  misc_feature 4: note = RNA
  misc_feature 5..15: note = DNA
  misc_feature 16: note = RNA
  misc_feature 17..20: note = DNA
  modified_base 1..8: mod_base = OTHER; note = 2' O-methyl or 2' methoxyethyl ribonucleotide
  modified_base 4: mod_base = OTHER; note = uracil
  modified_base 16: mod_base = OTHER; note = uracil
ccatactttccactgtaacg

SEQ ID NO: 201 - DNA, 20 bp, synthetic construct
  source 1..20: mol_type = other DNA
  modified_base 1..9: mod_base = OTHER; note = 2' O-methyl or 2' methoxyethyl ribonucleotide
tgctccgttggtgcttgttc

SEQ ID NO: 202 - DNA, 20 bp, synthetic construct
  source 1..20: mol_type = other DNA
  modified_base 1..8: mod_base = OTHER; note = 2' O-methyl or 2' methoxyethyl ribonucleotide
tgctccgttggtgcttgttc

SEQ ID NO: 203 - DNA, 20 bp, synthetic construct
  source 1..20: mol_type = other DNA
  modified_base 1..9: mod_base = OTHER; note = 2' O-methyl or 2' methoxyethyl ribonucleotide
tgctccgttggtgcttgttc

SEQ ID NO: 204 - DNA, 20 bp, synthetic construct
  source 1..20: mol_type = other DNA
  modified_base 1..8: mod_base = OTHER; note = 2' O-methyl or 2' methoxyethyl ribonucleotide
tgctccgttggtgcttgttc

SEQ ID NO: 205 - DNA, 20 bp, synthetic construct
  source 1..20: mol_type = other DNA
  modified_base 2: mod_base = OTHER; note = uracil
  modified_base 1..9: mod_base = OTHER; note = 2' O-methyl or 2' methoxyethyl ribonucleotide
  misc_feature 1: note = DNA
  misc_feature 2: note = RNA
  misc_feature 3..20: note = DNA
ctcagtaacattgacaccac

SEQ ID NO: 206 - DNA, 20 bp, synthetic construct
  source 1..20: mol_type = other DNA
  modified_base 2: mod_base = OTHER; note = uracil
  modified_base 1..8: mod_base = OTHER; note = 2' O-methyl or 2' methoxyethyl ribonucleotide
  misc_feature 1: note = DNA
  misc_feature 2: note = RNA
  misc_feature 3..20: note = DNA
ctcagtaacattgacaccac

SEQ ID NO: 207 - DNA, 20 bp, synthetic construct
  source 1..20: mol_type = other DNA
  modified_base 2: mod_base = OTHER; note = uracil
  modified_base 1..9: mod_base = OTHER; note = 2' O-methyl or 2' methoxyethyl ribonucleotide
  misc_feature 1: note = DNA
  misc_feature 2: note = RNA
  misc_feature 3..20: note = DNA
ctcagtaacattgacaccac

SEQ ID NO: 208 - DNA, 20 bp, synthetic construct
  source 1..20: mol_type = other DNA
  modified_base 2: mod_base = OTHER; note = uracil
  modified_base 1..8: mod_base = OTHER; note = 2' O-methyl or 2' methoxyethyl ribonucleotide
  misc_feature 1: note = DNA
  misc_feature 2: note = RNA
  misc_feature 3..20: note = DNA
ctcagtaacattgacaccac

SEQ ID NO: 209 - DNA, 20 bp, synthetic construct
  source 1..20: mol_type = other DNA
  modified_base 1..9: mod_base = OTHER; note = 2' O-methyl or 2' methoxyethyl ribonucleotide
gcagaggtgaagcgaagtgc

SEQ ID NO: 210 - DNA, 20 bp, synthetic construct
  source 1..20: mol_type = other DNA
  modified_base 1..8: mod_base = OTHER; note = 2' O-methyl or 2' methoxyethyl ribonucleotide
gcagaggtgaagcgaagtgc

SEQ ID NO: 211 - DNA, 20 bp, synthetic construct
  source 1..20: mol_type = other DNA
  modified_base 1..9: mod_base = OTHER; note = 2' O-methyl or 2' methoxyethyl ribonucleotide
gcagaggtgaagcgaagtgc

SEQ ID NO: 212 - DNA, 20 bp, synthetic construct
  source 1..20: mol_type = other DNA
  modified_base 1..8: mod_base = OTHER; note = 2' O-methyl or 2' methoxyethyl ribonucleotide
gcagaggtgaagcgaagtgc

SEQ ID NO: 213 - DNA, 16 bp, synthetic construct
  source 1..16: mol_type = other DNA
  modified_base 1..5: mod_base = OTHER; note = 2' O-methyl or 2' methoxyethyl ribonucleotide
gggcctgtttagtcgc

SEQ ID NO: 214 - DNA, 16 bp, synthetic construct
  source 1..16: mol_type = other DNA
  modified_base 1..3: mod_base = OTHER; note = 2' O-methyl or 2' methoxyethyl ribonucleotide
gggcctgtttagtcgc

SEQ ID NO: 215 - DNA, 16 bp, synthetic construct
  source 1..16: mol_type = other DNA
  modified_base 1..5: mod_base = OTHER; note = 2' O-methyl or 2' methoxyethyl ribonucleotide
gggcctgtttagtcgc

SEQ ID NO: 216 - DNA, 16 bp, synthetic construct
  source 1..16: mol_type = other DNA
  modified_base 1..3: mod_base = OTHER; note = 2' O-methyl or 2' methoxyethyl ribonucleotide
gggcctgtttagtcgc

SEQ ID NO: 217 - DNA, 16 bp, synthetic construct
  source 1..16: mol_type = other DNA
  modified_base 1..4: mod_base = OTHER; note = 2' O-methyl or 2' methoxyethyl ribonucleotide
gggcctgtttagtcgc

SEQ ID NO: 218 - DNA, 16 bp, synthetic construct
  source 1..16: mol_type = other DNA
  modified_base 1..3: mod_base = OTHER; note = 2' O-methyl or 2' methoxyethyl ribonucleotide
gggcctgtttagtcgc

SEQ ID NO: 219 - DNA, 20 bp, synthetic construct
  source 1..20: mol_type = other DNA
  modified_base 1..8: mod_base = OTHER; note = 2' O-methyl or 2' methoxyethyl ribonucleotide
acggcattggtgcacagttt

SEQ ID NO: 220 - DNA, 20 bp, synthetic construct
  source 1..20: mol_type = other DNA
  modified_base 1..9: mod_base = OTHER; note = 2' O-methyl or 2' methoxyethyl ribonucleotide
acggcattggtgcacagttt

SEQ ID NO: 221 - DNA, 20 bp, synthetic construct
  source 1..20: mol_type = other DNA
  modified_base 1..8: mod_base = OTHER; note = 2' O-methyl or 2' methoxyethyl ribonucleotide
acggcattggtgcacagttt

SEQ ID NO: 222 - DNA, 20 bp, synthetic construct
  source 1..20: mol_type = other DNA
  modified_base 1..9: mod_base = OTHER; note = 2' O-methyl or 2' methoxyethyl ribonucleotide
acggcattggtgcacagttt

SEQ ID NO: 223 - DNA, 20 bp, synthetic construct
  source 1..20: mol_type = other DNA
  modified_base 1..8: mod_base = OTHER; note = 2' O-methyl or 2' methoxyethyl ribonucleotide
ctttattccaaagggcagct

SEQ ID NO: 224 - DNA, 20 bp, synthetic construct
  source 1..20: mol_type = other DNA
  modified_base 1..9: mod_base = OTHER; note = 2' O-methyl or 2' methoxyethyl ribonucleotide
ctttattccaaagggcagct

SEQ ID NO: 225 - DNA, 20 bp, synthetic construct
  source 1..20: mol_type = other DNA
  modified_base 1..8: mod_base = OTHER; note = 2' O-methyl or 2' methoxyethyl ribonucleotide
ctttattccaaagggcagct

SEQ ID NO: 226 - DNA, 20 bp, synthetic construct
  source 1..20: mol_type = other DNA
  modified_base 1..9: mod_base = OTHER; note = 2' O-methyl or 2' methoxyethyl ribonucleotide
ctttattccaaagggcagct

SEQ ID NO: 227 - DNA, 20 bp, synthetic construct
  source 1..20: mol_type = other DNA
  modified_base 1..8: mod_base = OTHER; note = 2' O-methyl or 2' methoxyethyl ribonucleotide
ctttattccaaagggcagct

SEQ ID NO: 228 - DNA, 20 bp, synthetic construct
  source 1..20: mol_type = other DNA
  modified_base 1..9: mod_base = OTHER; note = 2' O-methyl or 2' methoxyethyl ribonucleotide
ctttattccaaagggcagct

SEQ ID NO: 229 - DNA, 20 bp, synthetic construct
  source 1..20: mol_type = other DNA
  modified_base 1..9: mod_base = OTHER; note = 2' O-methyl or 2' methoxyethyl ribonucleotide
cacaaacaagctggtcggtt

SEQ ID NO: 230 - DNA, 20 bp, synthetic construct
  source 1..20: mol_type = other DNA
  modified_base 1..8: mod_base = OTHER; note = 2' O-methyl or 2' methoxyethyl ribonucleotide
cacaaacaagctggtcggtt

SEQ ID NO: 231 - DNA, 20 bp, synthetic construct
  source 1..20: mol_type = other DNA
  modified_base 1..9: mod_base = OTHER; note = 2' O-methyl or 2' methoxyethyl ribonucleotide
cacaaacaagctggtcggtt

SEQ ID NO: 232 - DNA, 20 bp, synthetic construct
  source 1..20: mol_type = other DNA
  modified_base 1..8: mod_base = OTHER; note = 2' O-methyl or 2' methoxyethyl ribonucleotide
cacaaacaagctggtcggtt

SEQ ID NO: 233 - DNA, 18 bp, synthetic construct
  source 1..18: mol_type = other DNA
  modified_base 1..7: mod_base = OTHER; note = 2' O-methyl or 2' methoxyethyl ribonucleotide
ccgttttcttaccaccct

SEQ ID NO: 234 - DNA, 18 bp, synthetic construct
  source 1..18: mol_type = other DNA
  modified_base 1..6: mod_base = OTHER; note = 2' O-methyl or 2' methoxyethyl ribonucleotide
ccgttttcttaccaccct

SEQ ID NO: 235 - DNA, 18 bp, synthetic construct
  source 1..18: mol_type = other DNA
  modified_base 1..7: mod_base = OTHER; note = 2' O-methyl or 2' methoxyethyl ribonucleotide
ccgttttcttaccaccct

SEQ ID NO: 236 - DNA, 18 bp, synthetic construct
  source 1..18: mol_type = other DNA
  modified_base 1..6: mod_base = OTHER; note = 2' O-methyl or 2' methoxyethyl ribonucleotide
ccgttttcttaccaccct

SEQ ID NO: 237 - DNA, 19 bp, synthetic construct
  source 1..19: mol_type = other DNA
  modified_base 1..8: mod_base = OTHER; note = 2' O-methyl or 2' methoxyethyl ribonucleotide
atggtttattccatggcca

SEQ ID NO: 238 - DNA, 19 bp, synthetic construct
  source 1..19: mol_type = other DNA
  modified_base 1..8: mod_base = OTHER; note = 2' O-methyl or 2' methoxyethyl ribonucleotide
atggtttattccatggcca

SEQ ID NO: 239 - DNA, 19 bp, synthetic construct
  source 1..19: mol_type = other DNA
  modified_base 1..7: mod_base = OTHER; note = 2' O-methyl or 2' methoxyethyl ribonucleotide
atggtttattccatggcca

SEQ ID NO: 240 - DNA, 19 bp, synthetic construct
  source 1..19: mol_type = other DNA
  modified_base 1..7: mod_base = OTHER; note = 2' O-methyl or 2' methoxyethyl ribonucleotide
atggtttattccatggcca

SEQ ID NO: 241 - DNA, 21 bp, synthetic construct
  source 1..21: mol_type = other DNA
  modified_base 2..4: mod_base = OTHER; note = uracil
  modified_base 1..10: mod_base = OTHER; note = 2' O-methyl or 2' methoxyethyl ribonucleotide
ctttcagacacgggaacacgg

SEQ ID NO: 242 - DNA, 21 bp, synthetic construct
  source 1..21: mol_type = other DNA
  modified_base 2..4: mod_base = OTHER; note = uracil
  modified_base 1..9: mod_base = OTHER; note = 2' O-methyl or 2' methoxyethyl ribonucleotide
ctttcagacacgggaacacgg

SEQ ID NO: 243 - DNA, 21 bp, synthetic construct
  source 1..21: mol_type = other DNA
  modified_base 2..4: mod_base = OTHER; note = uracil
  modified_base 1..10: mod_base = OTHER; note = 2' O-methyl or 2' methoxyethyl ribonucleotide
ctttcagacacgggaacacgg

SEQ ID NO: 244 - DNA, 21 bp, synthetic construct
  source 1..21: mol_type = other DNA
  modified_base 2..4: mod_base = OTHER; note = uracil
  modified_base 1..9: mod_base = OTHER; note = 2' O-methyl or 2' methoxyethyl ribonucleotide
ctttcagacacgggaacacgg

SEQ ID NO: 245 - DNA, 21 bp, synthetic construct
  source 1..21: mol_type = other DNA
  modified_base 2..4: mod_base = OTHER; note = uracil
  modified_base 1..10: mod_base = OTHER; note = 2' O-methyl or 2' methoxyethyl ribonucleotide
ctttcagacacgggaacacgg

SEQ ID NO: 246 - DNA, 21 bp, synthetic construct
  source 1..21: mol_type = other DNA
  modified_base 2..4: mod_base = OTHER; note = uracil
  modified_base 1..9: mod_base = OTHER; note = 2' O-methyl or 2' methoxyethyl ribonucleotide
ctttcagacacgggaacacgg

SEQ ID NO: 247 - DNA, 20 bp, synthetic construct
  source 1..20: mol_type = other DNA
  modified_base 1..9: mod_base = OTHER; note = 2' O-methyl or 2' methoxyethyl ribonucleotide
ggacattgccagtaatcgca

SEQ ID NO: 248 - DNA, 20 bp, synthetic construct
  source 1..20: mol_type = other DNA
  modified_base 1..8: mod_base = OTHER; note = 2' O-methyl or 2' methoxyethyl ribonucleotide
ggacattgccagtaatcgca

SEQ ID NO: 249 - DNA, 20 bp, synthetic construct
  source 1..20: mol_type = other DNA
  modified_base 1..9: mod_base = OTHER; note = 2' O-methyl or 2' methoxyethyl ribonucleotide
ggacattgccagtaatcgca

SEQ ID NO: 250 - DNA, 20 bp, synthetic construct
  source 1..20: mol_type = other DNA
  modified_base 1..8: mod_base = OTHER; note = 2' O-methyl or 2' methoxyethyl ribonucleotide
ggacattgccagtaatcgca

SEQ ID NO: 251 - DNA, 20 bp, synthetic construct
  source 1..20: mol_type = other DNA
  modified_base 1..9: mod_base = OTHER; note = 2' O-methyl or 2' methoxyethyl ribonucleotide
cttcacatttcgatccaaca

SEQ ID NO: 252 - DNA, 20 bp, synthetic construct
  source 1..20: mol_type = other DNA
  modified_base 1..8: mod_base = OTHER; note = 2' O-methyl or 2' methoxyethyl ribonucleotide
cttcacatttcgatccaaca

SEQ ID NO: 253 - DNA, 20 bp, synthetic construct
  source 1..20: mol_type = other DNA
  modified_base 1..9: mod_base = OTHER; note = 2' O-methyl or 2' methoxyethyl ribonucleotide
cttcacatttcgatccaaca

SEQ ID NO: 254 - DNA, 20 bp, synthetic construct
  source 1..20: mol_type = other DNA
  modified_base 1..8: mod_base = OTHER; note = 2' O-methyl or 2' methoxyethyl ribonucleotide
cttcacatttcgatccaaca

SEQ ID NO: 255 - DNA, 18 bp, synthetic construct
  source 1..18: mol_type = other DNA
  modified_base 1..14: mod_base = OTHER; note = 2' O-methyl or 2' methoxyethyl ribonucleotide
agttggagcaagattatc

SEQ ID NO: 256 - DNA, 18 bp, synthetic construct
  source 1..18: mol_type = other DNA
  modified_base 1..14: mod_base = OTHER; note = 2' O-methyl or 2' methoxyethyl ribonucleotide
agttggagcaagattatc

SEQ ID NO: 257 - DNA, 18 bp, synthetic construct
  source 1..18: mol_type = other DNA
  modified_base 1..14: mod_base = OTHER; note = 2' O-methyl or 2' methoxyethyl ribonucleotide
agttggagcaagattatc

SEQ ID NO: 258 - DNA, 22 bp, synthetic construct
  source 1..22: mol_type = other DNA
  modified_base 1..18: mod_base = OTHER; note = 2' O-methyl or 2' methoxyethyl ribonucleotide
caatgccatcctggagttcctg

SEQ ID NO: 259 - DNA, 22 bp, synthetic construct
  source 1..22: mol_type = other DNA
  modified_base 1..18: mod_base = OTHER; note = 2' O-methyl or 2' methoxyethyl ribonucleotide
caatgccatcctggagttcctg

SEQ ID NO: 260 - DNA, 25 bp, synthetic construct
  source 1..25: mol_type = other DNA
  modified_base 1..21: mod_base = OTHER; note = 2' O-methyl or 2' methoxyethyl ribonucleotide
gttgcctccggttctgaaggtgttc

SEQ ID NO: 261 - DNA, 25 bp, synthetic construct
  source 1..25: mol_type = other DNA
  modified_base 1..21: mod_base = OTHER; note = 2' O-methyl or 2' methoxyethyl ribonucleotide
gttgcctccggttctgaaggtgttc

SEQ ID NO: 262 - DNA, 30 bp, synthetic construct; identical to SEQ ID NO: 104
  source 1..30: mol_type = other DNA
  modified_base 1..26: mod_base = OTHER; note = 2' O-methyl or 2' methoxyethyl ribonucleotide

SEQ ID NO: 263 - DNA, 30 bp, synthetic construct; identical to SEQ ID NO: 104
  source 1..30: mol_type = other DNA
  modified_base 1..26: mod_base = OTHER; note = 2' O-methyl or 2' methoxyethyl ribonucleotide

SEQ ID NO: 264-267 - skipped (4 entries)

SEQ ID NO: 268 - DNA, 20 bp, synthetic construct
  source 1..20: mol_type = other DNA
  modified_base 1: mod_base = OTHER; note = 2' O-methyl ribonucleotide
  modified_base 2: mod_base = OTHER; note = 2' methoxyethyl ribonucleotide
  modified_base 3: mod_base = OTHER; note = 2' O-methyl ribonucleotide
  modified_base 4: mod_base = OTHER; note = 2' methoxyethyl ribonucleotide
  modified_base 5: mod_base = OTHER; note = 2' O-methyl ribonucleotide
  modified_base 6: mod_base = OTHER; note = 2' methoxyethyl ribonucleotide
  modified_base 7..8: mod_base = OTHER; note = 2' O-methyl ribonucleotide
caggatacatttctacagct

SEQ ID NO: 269 - DNA, 20 bp, synthetic construct
  source 1..20: mol_type = other DNA
  modified_base 1: mod_base = OTHER; note = 2' O-methyl ribonucleotide
  modified_base 2: mod_base = OTHER; note = 2' methoxyethyl ribonucleotide
  modified_base 3: mod_base = OTHER; note = 2' O-methyl ribonucleotide
  modified_base 4: mod_base = OTHER; note = 2' methoxyethyl ribonucleotide
  modified_base 5: mod_base = OTHER; note = 2' O-methyl ribonucleotide
  modified_base 6: mod_base = OTHER; note = 2' methoxyethyl ribonucleotide
  modified_base 7: mod_base = OTHER; note = 2' O-methyl ribonucleotide
caggatacatttctacagct

SEQ ID NO: 270-279 - skipped (10 entries)

SEQ ID NO: 280 - DNA, 20 bp, synthetic construct
  source 1..20: mol_type = other DNA
gcctcagtctgcttcgcacc

SEQ ID NO: 281 - DNA, 20 bp, synthetic construct
  source 1..20: mol_type = other DNA
tcttggttacatgaaatccc

SEQ ID NO: 282 - DNA, 20 bp, synthetic construct
  source 1..20: mol_type = other DNA
agcttcttgtccagctttat

SEQ ID NO: 283 - DNA, 20 bp, synthetic construct
  source 1..20: mol_type = other DNA
gctccttccactgatcctgc

SEQ ID NO: 284 - DNA, 20 bp, synthetic construct
  source 1..20: mol_type = other DNA
ccttggccacgccggcatcc

SEQ ID NO: 285 - DNA, 16 bp, synthetic construct
  source 1..16: mol_type = other DNA
ctatttggatgtcagc

SEQ ID NO: 286 - DNA, 20 bp, synthetic construct
  source 1..20: mol_type = other DNA
ggacattgccagtaatcgca

SEQ ID NO: 287 - DNA, 20 bp, synthetic construct
  source 1..20: mol_type = other DNA
tgcaagtctcttggcaaaca

SEQ ID NO: 288 - DNA, 20 bp, synthetic construct
  source 1..20: mol_type = other DNA
tgccatttaatgagcttcac

SEQ ID NO: 289 - DNA, 18 bp, synthetic construct
  source 1..18: mol_type = other DNA
tcactttcataatgctgg

SEQ ID NO: 290 - DNA, 20 bp, synthetic construct
  source 1..20: mol_type = other DNA
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 291 - DNA, 20 bp, synthetic construct
  source 1..20: mol_type = other DNA
tgctccgttggtgcttgttc

SEQ ID NO: 292 - DNA, 20 bp, synthetic construct
  source 1..20: mol_type = other DNA
  modified_base 2: mod_base = OTHER; note = uracil
  misc_feature 1: note = DNA
  misc_feature 2: note = RNA
  misc_feature 3..20: note = DNA
ctcagtaacattgacaccac

SEQ ID NO: 293 - DNA, 20 bp, synthetic construct
  source 1..20: mol_type = other DNA
gcagaggtgaagcgaagtgc

SEQ ID NO: 294 - DNA, 16 bp, synthetic construct
  source 1..16: mol_type = other DNA
gggcctgtttagtcgc

SEQ ID NO: 295 - DNA, 20 bp, synthetic construct
  source 1..20: mol_type = other DNA
acggcattggtgcacagttt

SEQ ID NO: 296 - DNA, 20 bp, synthetic construct
  source 1..20: mol_type = other DNA
ctttattccaaagggcagct

SEQ ID NO: 297 - DNA, 20 bp, synthetic construct
  source 1..20: mol_type = other DNA
cacaaacaagctggtcggtt

SEQ ID NO: 298 - DNA, 18 bp, synthetic construct
  source 1..18: mol_type = other DNA
ccgttttcttaccaccct

SEQ ID NO: 299 - DNA, 19 bp, synthetic construct
  source 1..19: mol_type = other DNA
atggtttattccatggcca

SEQ ID NO: 300 - DNA, 21 bp, synthetic construct
  source 1..21: mol_type = other DNA
  modified_base 2..4: mod_base = OTHER; note = uracil
  misc_feature 1: note = DNA
  misc_feature 2..4: note = RNA
  misc_feature 5..21: note = DNA
ctttcagacacgggaacacgg

SEQ ID NO: 301 - DNA, 20 bp, synthetic construct
  source 1..20: mol_type = other DNA
cttcacatttcgatccaaca

SEQ ID NO: 302 - DNA, 18 bp, synthetic construct
  source 1..18: mol_type = other DNA
agttggagcaagattatc

SEQ ID NO: 303 - DNA, 22 bp, synthetic construct
  source 1..22: mol_type = other DNA
caatgccatcctggagttcctg

SEQ ID NO: 304 - DNA, 25 bp, synthetic construct
  source 1..25: mol_type = other DNA
gttgcctccggttctgaaggtgttc

SEQ ID NO: 305 - DNA, 30 bp, synthetic construct; identical to SEQ ID NO: 104
  source 1..30: mol_type = other DNA

SEQ ID NO: 306 - DNA, 20 bp, synthetic construct
  source 1..20: mol_type = other DNA
caggatacatttctacagct

SEQ ID NO: 307 - DNA, 20 bp, synthetic construct
  source 1..20: mol_type = other DNA
  modified_base 1..9: mod_base = OTHER; note = 2' methoxyethyl ribonucleotide
ctcagtaacattgacaccac

SEQ ID NO: 308 - DNA, 20 bp, synthetic construct
  source 1..20: mol_type = other DNA
  modified_base 1..8: mod_base = OTHER; note = 2' methoxyethyl ribonucleotide
ctcagtaacattgacaccac

SEQ ID NO: 309 - DNA, 20 bp, synthetic construct
  source 1..20: mol_type = other DNA
  modified_base 1..9: mod_base = OTHER; note = 2' methoxyethyl ribonucleotide
ctcagtaacattgacaccac

SEQ ID NO: 310 - DNA, 20 bp, synthetic construct
  source 1..20: mol_type = other DNA
  modified_base 1..11: mod_base = OTHER; note = 2' methoxyethyl ribonucleotide
ctcagtaacattgacaccac

SEQ ID NO: 311 - DNA, 20 bp, synthetic construct
  source 1..20: mol_type = other DNA
  modified_base 1..9: mod_base = OTHER; note = 2' methoxyethyl ribonucleotide
ctcagtaacattgacaccac

SEQ ID NO: 312 - DNA, 20 bp, synthetic construct
  source 1..20: mol_type = other DNA
  modified_base 1..9: mod_base = OTHER; note = 2' methoxyethyl ribonucleotide
ctcagtaacattgacaccac

SEQ ID NO: 313 - DNA, 20 bp, synthetic construct
  source 1..20: mol_type = other DNA
  modified_base 1..9: mod_base = OTHER; note = 2' methoxyethyl ribonucleotide
ctcagtaacattgacaccac

SEQ ID NO: 314 - DNA, 20 bp, synthetic construct
  source 1..20: mol_type = other DNA
  modified_base 1..9: mod_base = OTHER; note = 2' methoxyethyl ribonucleotide
ctcagtaacattgacaccac

SEQ ID NO: 315 - DNA, 18 bp, synthetic construct
  source 1..18: mol_type = other DNA
  modified_base 1..7: mod_base = OTHER; note = 2' methoxyethyl ribonucleotide
ccgttttcttaccaccct

SEQ ID NO: 316 - DNA, 18 bp, synthetic construct
  source 1..18: mol_type = other DNA
  modified_base 1..7: mod_base = OTHER; note = 2' methoxyethyl ribonucleotide
ccgttttcttaccaccct

SEQ ID NO: 317 - DNA, 20 bp, synthetic construct
  source 1..20: mol_type = other DNA
  modified_base 1..8: mod_base = OTHER; note = 2' methoxyethyl ribonucleotide
caggatacatttctacagct

SEQ ID NO: 318 - DNA, 20 bp, synthetic construct
  source 1..20: mol_type = other DNA
  modified_base 1..8: mod_base = OTHER; note = 2' methoxyethyl ribonucleotide
caggatacatttctacagct

SEQ ID NO: 319 - DNA, 20 bp, synthetic construct
  source 1..20: mol_type = other DNA
  modified_base 1..8: mod_base = OTHER; note = 2' methoxyethyl ribonucleotide
caggatacatttctacagct

SEQ ID NO: 320 - DNA, 26 bp, synthetic construct
  source 1..26: mol_type = other DNA
  modified_base 1..5: mod_base = OTHER; note = 2' methoxyethyl ribonucleotide
  modified_base 16..20: mod_base = OTHER; note = 2' methoxyethyl ribonucleotide
caggatacatttctacagctacagct

SEQ ID NO: 321 - DNA, 26 bp, synthetic construct
  source 1..26: mol_type = other DNA
  modified_base 1..8: mod_base = OTHER; note = 2' methoxyethyl ribonucleotide
caggatacatttctacagctacagct

SEQ ID NO: 322 - DNA, 26 bp, synthetic construct
  source 1..26: mol_type = other DNA
  modified_base 1..8: mod_base = OTHER; note = 2' methoxyethyl ribonucleotide
caggatacatttctacagctacagct

SEQ ID NO: 323 - DNA, 26 bp, synthetic construct
  source 1..26: mol_type = other DNA
  modified_base 1..9: mod_base = OTHER; note = 2' methoxyethyl ribonucleotide
caggatacatttctacagctagcagg

SEQ ID NO: 324 - DNA, 26 bp, synthetic construct
  source 1..26: mol_type = other DNA
  modified_base 1..9: mod_base = OTHER; note = 2' methoxyethyl ribonucleotide
caggatacatttctacagctagcagg

SEQ ID NO: 325 - DNA, 20 bp, synthetic construct
  source 1..20: mol_type = other DNA
  modified_base 1..5: mod_base = OTHER; note = 2'MOE phosphorothioate
  modified_base 16..20: mod_base = OTHER; note = 2'MOE phosphorothioate
caggatacatttctacagct

SEQ ID NO: 326 - DNA, 27 bp, synthetic construct
  source 1..27: mol_type = other DNA
  modified_base 1..5: mod_base = OTHER; note = 2' methoxyethyl ribonucleotide
  modified_base 16..20: mod_base = OTHER; note = 2' methoxyethyl ribonucleotide
caggatacatttctacagcttagcagg

SEQ ID NO: 327 - DNA, 20 bp, synthetic construct
  source 1..20: mol_type = other DNA
  modified_base 1..9: mod_base = OTHER; note = 2' methoxyethyl ribonucleotide
gcttcttgtccagctttatt

SEQ ID NO: 328 - DNA, 20 bp, synthetic construct
  source 1..20: mol_type = other DNA
  modified_base 1..9: mod_base = OTHER; note = 2' methoxyethyl ribonucleotide
gcttcttgtccagctttatt

SEQ ID NO: 329 - DNA, 20 bp, synthetic construct
  source 1..20: mol_type = other DNA
  modified_base 1..4: mod_base = OTHER; note = 2' methoxyethyl ribonucleotide
  modified_base 6..9: mod_base = OTHER; note = 2' methoxyethyl ribonucleotide
gcttcttgtccagctttatt

SEQ ID NO: 330 - DNA, 25 bp, synthetic construct
  source 1..25: mol_type = other DNA
  modified_base 1..9: mod_base = OTHER; note = 2' methoxyethyl ribonucleotide
gcttcttgtccagctttattgggag

SEQ ID NO: 331 - DNA, 25 bp, synthetic construct
  source 1..25: mol_type = other DNA
  modified_base 1..9: mod_base = OTHER; note = 2' methoxyethyl ribonucleotide
gcttcttgtccagctttattgggag

SEQ ID NO: 332-336 - skipped (5 entries)

SEQ ID NO: 337 - DNA, 27 bp, synthetic construct
  source 1..27: mol_type = other DNA
  modified_base 1..5: mod_base = OTHER; note = 2' methoxyethyl ribonucleotide
  modified_base 16..20: mod_base = OTHER; note = 2' methoxyethyl ribonucleotide
caggatacatttctacagcttagcagg

SEQ ID NO: 338 - DNA, 27 bp, synthetic construct
  source 1..27: mol_type = other DNA
  modified_base 1..5: mod_base = OTHER; note = 2' methoxyethyl ribonucleotide
  modified_base 16..20: mod_base = OTHER; note = 2' methoxyethyl ribonucleotide
caggatacatttctacagcttagcagg

SEQ ID NO: 339 - DNA, 20 bp, synthetic construct
  source 1..20: mol_type = other DNA
  modified_base 1..5: mod_base = OTHER; note = 2' methoxyethyl ribonucleotide
  modified_base 16..20: mod_base = OTHER; note = 2' methoxyethyl ribonucleotide
ccaaatcttataataactac

SEQ ID NO: 340 - DNA, 20 bp, synthetic construct
  source 1..20: mol_type = other DNA
  modified_base 1..8: mod_base = OTHER; note = 2' methoxyethyl ribonucleotide
ccaaatcttataataactac